(19) United States
(12) Patent Application Publication
(10) Pub. No.: US 2025/0263765 A1
(43) Pub. Date: Aug. 21, 2025

(54) METHODS FOR PRODUCING NUCLEIC ACIDS

(71) Applicant: PFIZER INC., NEW YORK, NY (US)

(72) Inventors: Amer Al-Lozi, St. Louis, MO (US); Keshab Bhattacharya, Brentwood, MO (US); Guogang Dong, Winchester, MA (US); Swapnadip Ghosh, Chesterfield, MO (US); Hugo Jhun, Bronx, NY (US); Justin Keith Moran, West Nyack, NY (US); Urmi Ankit Patel, Wallington, NJ (US); Jennifer Julia Quaglia Motta, Somerville, MA (US); Joseph Russo, Ballwin, MO (US); Tatenda Shopera, St. Louis, MO (US); Carrie Leanne Simms, St. Louis, MO (US); Dillon Jarod Sinanan, Somerville, MA (US); Michael Casey Vande Voorde, St. Louis, MO (US); Jenna Kathryn Williamson, Chesterfield, MO (US); Nichole Lea Wood, Chesterfield, MO (US); Xiaolu Zheng, Clayton, MO (US); Martin John Allen, Webster Groves, MO (US); Daniel Ward Allison, Wildwood, MO (US); Kaley Elizabeth Armour, Troy, IL (US); Shelby Hutchins Breen, North Andover, MA (US); Sun Joong Kim, Chesterfield, MO (US); Athreya Subramanian Ramesh, Lincoln, MA (US); Erin Courtney Reeves, St. Louis, MO (US); Karenna Caroline Smith, St. Louis, MO (US); Michael Jay Swyers, Saint Peters, MO (US)

(73) Assignee: PFIZER INC., NEW YORK, NY (US)

(21) Appl. No.: 18/856,566

(22) PCT Filed: May 3, 2023
(86) PCT No.: PCT/IB2023/054620

§ 371 Date: Oct. 11, 2024

Related U.S. Application Data
(60) Provisional application No. 63/338,878, filed on May 5, 2022.
(60) Provisional application No. 63/499,276, filed on May 1, 2023.

(51) Int. Cl. C12P 19/34

(52) U.S. Cl. CPC C12P 19/34

(57) ABSTRACT

Described are methods for producing RNA molecules in an in vitro transcription reaction. Also described are methods for producing an mRNA molecule from a circular double-stranded DNA template using an in vitro transcription reaction system wherein the in vitro transcription reaction system lacks a polyamine. Also described are in vitro transcription reaction systems comprising enzymatic 5′ capping and oligo (dT) purification.

### CROSS-REFERENCE TO RELATED APPLICATIONS

This application claims the benefit under 35 U.S.C. § 119 (e) of U.S. Provisional Patent Application Ser. No. 63/499,276, filed May 1, 2023, and U.S. Provisional Patent Application Ser. No. 63/338,878, filed May 5, 2022, the disclosures of which are hereby incorporated by reference in their entirety.

### FIELD

The present invention relates to an improved process for synthesis of nucleic acid molecules, in particular cell-free enzymatic synthesis of DNA and synthesis of RNA molecules via in vitro transcription (IVT).

### BACKGROUND

There is a need in the art for improved and efficient in vitro methods of nucleic acid amplification, specifically DNA molecules and templates for IVT, without the use of plasmids and bacterial fermentation (e.g., cell-free production of DNA molecules). Such improved methods may, for example, facilitate faster response times to address virus strain changes during a pandemic or seasonal viral changes, since conventional multi-step methods of DNA-template generation from plasmids produced by bacteria fermentation can be time-consuming.

### SUMMARY OF THE INVENTION

In some embodiments, disclosed herein are methods of producing an mRNA molecule comprising: (a) obtaining a composition comprising: a circular double-stranded DNA (dsDNA) template, a primer, a deoxyribonucleotide triphosphate (dNTP), and a DNA polymerase; (b) incubating the composition for a time period between about three and twenty-four hours at a temperature between about 30° C. and 45° C. to obtain an incubated composition; and (c) contacting the incubated composition with an in vitro transcription reaction system comprising an RNA polymerase and ribonucleotides under conditions sufficient for in vitro transcription to produce the mRNA molecule, wherein the contacting occurs for between about 120 minutes and about 260 minutes, wherein the contacting occurs at a temperature between 25° C. and 45° C., wherein the in vitro transcription reaction system further comprises a buffer, a magnesium ion, and a reducing agent, and wherein the in vitro transcription reaction system lacks a polyamine. In some embodiments, the methods further comprise contacting the incubated composition with a restriction endonuclease to obtain a digested composition prior to contacting the incubated composition with the in vitro transcription reaction system. In some embodiments, contacting the incubated composition with the restriction endonuclease occurs in a same reaction vessel as incubating the composition. In some embodiments, contacting the incubated composition with the in vitro transcription reaction occurs in the same reaction vessel as incubating the composition. In some embodiments, the methods of producing an mRNA molecule disclosed herein do not comprise a heat denaturation reaction prior to incubating the composition. In some embodiments, the methods of producing an mRNA molecule disclosed herein do not comprise a heat inactivation reaction prior to performing the in vitro transcription reaction. In some embodiments, the methods of producing an mRNA molecule disclosed herein further comprise: (d) capturing the mRNA molecule using a capture method selected from the group consisting of an oligo (dT) magnetic bead, a resin, and a monolith.

In some embodiments, disclosed herein are methods of producing an mRNA molecule comprising: (a) obtaining a composition comprising: a circular double-stranded DNA (dsDNA) template, a primer, a deoxyribonucleotide triphosphate (dNTP), and a DNA polymerase; (b) incubating the composition for a time period between about three and eight hours at a temperature between about 30° C. and 45° C. to obtain an incubated composition; and (c) contacting the incubated composition with an in vitro transcription reaction system comprising an RNA polymerase and ribonucleotides under conditions sufficient for in vitro transcription to produce the mRNA molecule, wherein the contacting occurs for between about 120 minutes and about 260 minutes, wherein the contacting occurs at a temperature between 25° C. and 45° C., wherein the in vitro transcription reaction system further comprises a buffer, a magnesium ion, and a reducing agent, and wherein the in vitro transcription reaction system lacks a polyamine. In some embodiments, the time period is between about eighteen and twenty-four hours. In some embodiments, disclosed herein are methods of producing an mRNA molecule comprising: (a) obtaining a composition comprising: a circular double-stranded DNA (dsDNA) template, a primer, a deoxyribonucleotide triphosphate (dNTP), and a DNA polymerase; (b) incubating the composition for a time period between about three and twenty-four hours at a temperature between about 30° C. and 45° C. to obtain an incubated composition; and (c) contacting the incubated composition with an in vitro transcription reaction system comprising an RNA polymerase and ribonucleotides under conditions sufficient for in vitro transcription to produce the mRNA molecule, wherein the contacting occurs for between about 120 minutes and about 260 minutes, wherein the contacting occurs at a temperature between 25° C. and 45° C., wherein the in vitro transcription reaction system further comprises a buffer, a magnesium ion, and a reducing agent, wherein the in vitro transcription reaction system lacks a polyamine, and wherein the DNA polymerase is a phi29 DNA polymerase.

In some embodiments, disclosed herein are methods of producing an mRNA molecule comprising: (a) obtaining a composition comprising: a circular double-stranded DNA (dsDNA) template, a primer, a deoxyribonucleotide triphosphate (dNTP), and a DNA polymerase; (b) incubating the composition for a time period between about three and twenty-four hours at a temperature between about 30° C. and 45° C. to obtain an incubated composition; and (c) contacting the incubated composition with an in vitro transcription reaction system comprising an RNA polymerase and ribonucleotides under conditions sufficient for in vitro transcription to produce the mRNA molecule, wherein the contacting occurs for between about 120 minutes and about 260 minutes, wherein the contacting occurs at a temperature between 25° C. and 45° C., wherein the in vitro transcription reaction system further comprises a buffer, a magnesium ion, and a reducing agent, wherein the in vitro transcription reaction system lacks a polyamine, and wherein contacting further comprises supplementing the in vitro transcription reaction system with ribonucleotides. In some embodiments, the supplementing comprises continuous feeding of ribonucleotides. In some embodiments, the supplementing comprises semi-continuous feeding of ribonucleotides. In some embodiments, the supplementing comprises bolus feeding of ribonucleotides.

In some embodiments, disclosed herein are methods of producing an mRNA molecule comprising: (a) obtaining a composition comprising: a circular double-stranded DNA (dsDNA) template, a primer, a deoxyribonucleotide triphosphate (dNTP), and a DNA polymerase; (b) incubating the composition for a time period between about three and twenty-four hours at a temperature between about 30° C. and 45° C. to obtain an incubated composition; and (c) contacting the incubated composition with an in vitro transcription reaction system comprising an RNA polymerase and ribonucleotides under conditions sufficient for in vitro transcription to produce the mRNA molecule, wherein the contacting occurs for between about 120 minutes and about 260 minutes, wherein the contacting occurs at a temperature between 25° C. and 45° C., wherein the in vitro transcription reaction system further comprises a buffer, a magnesium ion, and a reducing agent, wherein the in vitro transcription reaction system lacks a polyamine, and wherein contacting further comprises supplementing the in vitro transcription reaction system with a magnesium ion. In some embodiments, the supplementing is selected from the group consisting of continuous feeding, semi-continuous feeding, and bolus feeding of a magnesium ion. In some embodiments, the contacting further comprises agitation. In some embodiments, the agitation is selected from the group consisting of a power/volume between about 1.3 and about 71.7 W/m³, a mixing time between about 1.3 and about 12.1 seconds, and an impeller tip speed between about 0.1 and about 0.4 m/s.

In some embodiments, disclosed herein are methods of producing an mRNA molecule comprising: (a) obtaining a composition comprising: a circular double-stranded DNA (dsDNA) template, a primer, a deoxyribonucleotide triphosphate (dNTP), and a DNA polymerase; (b) incubating the composition for a time period between about three and twenty-four hours at a temperature between about 30° C. and 45° C. to obtain an incubated composition; and (c) contacting the incubated composition with an in vitro transcription reaction system comprising an RNA polymerase and ribonucleotides under conditions sufficient for in vitro transcription to produce the mRNA molecule, wherein the contacting occurs for between about 120 minutes and about 260 minutes, wherein the contacting occurs at a temperature between 25° C. and 45° C., wherein the in vitro transcription reaction system further comprises a buffer, a magnesium ion, and a reducing agent, wherein the in vitro transcription reaction system lacks a polyamine, and wherein the in vitro transcription reaction system further comprises pyrophosphatase. In some embodiments, the in vitro transcription reaction system further comprises a ribonuclease inhibitor. In some embodiments, the in vitro transcription reaction system further comprises an acetate ion. In some embodiments, the buffer is selected from the group consisting of TRIS and HEPES. In some embodiments, the ribonucleotides are present in an amount between about 16 and about 50 mM. In some embodiments, the RNA polymerase is present in an amount between about 4000 and about 12000 U/mL. In some embodiments, the pyrophosphatase is present in an amount between about 0.25 and about 8.0 U/mL. In some embodiments, the buffer comprises a starting pH between about 7.5 and about 8.5. In some embodiments, the magnesium ion is present in an amount between about 12.8 and about 110 mM. In some embodiments, the magnesium ion and the ribonucleotides are present in a magnesium:ribonucleotide ratio of about 0.8 to about 2.2 mM Mg/mM NTP. In some embodiments, the buffer does not contain dithiothreitol (DTT).

In some embodiments, disclosed herein are methods of producing an mRNA molecule comprising: (a) obtaining a composition comprising: a plurality of linear double-stranded DNA (dsDNA) fragments, a 5′ exonuclease, a DNA polymerase, and a DNA ligase; (b) incubating the composition at a temperature between about 45° C. and 55° C. to obtain a circularized dsDNA template; and (c) contacting the circularized dsDNA template with a rolling circle amplification (RCA) reaction mix comprising: a primer or a primase enzyme, a deoxyribonucleotide triphosphate (dNTP), and a DNA polymerase, to obtain an amplified template; (d) contacting the amplified template with a restriction enzyme to obtain a digested template; and (e) contacting the digested template with an in vitro transcription reaction system comprising an RNA polymerase and ribonucleotides under conditions sufficient for in vitro transcription to produce the mRNA molecule, wherein the contacting occurs for between about 120 minutes and about 260 minutes, wherein the contacting occurs at a temperature between 25° C. and 45° C., wherein the in vitro transcription reaction system further comprises a buffer and a magnesium ion, and wherein steps (a) through (e) are performed sequentially in a single reaction vessel. In some embodiments, the circularized dsDNA template is present at a concentration of at least 0.5 ng per mL of RCA reaction mix. In some embodiments, the plurality of linear dsDNA fragments comprises a gene of interest, a promoter sequence, a 5′ UTR, a 3′ UTR, and a poly A sequence. In some embodiments, the gene of interest encodes an RNA molecule between 1.0 kb and 12.0 kb. In some embodiments, the gene of interest lacks homopolymer sequences greater than 5 base pairs. In some embodiments, the mRNA molecule is capped following in vitro transcription. In some embodiments, the mRNA molecule is capped enzymatically by a vaccinia virus capping enzyme. In some embodiments, the mRNA molecule is incubated at a temperature of 30° C. or lower for 60 minutes or less prior to capping by the vaccinia virus capping enzyme.

In some embodiments, disclosed herein are methods of producing an mRNA molecule comprising: (a) obtaining a composition comprising: a plurality of linear double-stranded DNA (dsDNA) fragments, a 5′ exonuclease, a DNA polymerase, and a DNA ligase; (b) incubating the composition at a temperature between about 45° C. and 55° C. to obtain a circularized dsDNA template; and (c) contacting the circularized dsDNA template with a rolling circle amplification (RCA) reaction mix comprising: a primer or a primase enzyme, a deoxyribonucleotide triphosphate (dNTP), and a DNA polymerase, to obtain an amplified template; (d) contacting the amplified template with a restriction enzyme to obtain a digested template; and (e) contacting the digested template with an in vitro transcription reaction system comprising an RNA polymerase and ribonucleotides under conditions sufficient for in vitro transcription to produce the mRNA molecule, wherein the contacting occurs for between about 120 minutes and about 260 minutes, wherein the contacting occurs at a temperature between 25° C. and 45° C., wherein the in vitro transcription reaction system further comprises a buffer and a magnesium ion, wherein steps (a) through (e) are performed sequentially in a single reaction vessel, and wherein contacting the digested template with an in vitro transcription reaction system further comprises supplementing the in vitro transcription reaction system with ribonucleotides, wherein supplementing comprises increasing a concentration of an individual ribonucleotide from an initial amount to a final amount. In some embodiments, supplementing comprises a method selected from the group consisting of continuous feeding of ribonucleotides, semi-continuous feeding of ribonucleotides, and bolus feeding of ribonucleotides. In some embodiments, the initial amount is between 1 mM and 11 mM and the final amount is between 11 mM and 26 mM. In some embodiments, the initial amount of ATP is about 11 mM, the initial amount of CTP is about 9 mM, the initial amount of GTP is about 1 mM, and the initial amount of pUTP is about 4 mM. In some embodiments, the final amount of ATP is about 24 mM, the final amount of CTP is about 22 mM, the final amount of GTP is about 16 mM, and the final amount of pUTP is about 13 mM. In some embodiments, the supplementing further comprises bolus feeding of ribonucleotides every five minutes of the in vitro transcription reaction. In some embodiments, contacting the digested template with an in vitro transcription reaction system further comprises supplementing the in vitro transcription reaction system with a magnesium ion, wherein supplementing comprises increasing a concentration of the magnesium ion from an initial amount to a final amount. In some embodiments, supplementing comprises a method selected from the group consisting of continuous feeding of a magnesium ion, semi-continuous feeding of a magnesium ion, and bolus feeding of a magnesium ion. In some embodiments, supplementing the in vitro transcription reaction system with a magnesium ion further comprises at least four magnesium ion additions during the in vitro transcription reaction. In some embodiments, the initial amount of the magnesium ion is about 25 mM and the final amount of the magnesium ion is about 60 mM. In some embodiments, the supplementing further comprises bolus feeding of the magnesium ion every 15 minutes for the first 60 minutes of the in vitro transcription reaction.

In some embodiments, the methods for producing an mRNA molecule disclosed herein comprise capturing an mRNA molecule produced by an in vitro transcription reaction using a capture method selected from the group consisting of an oligo (dT) magnetic bead, a resin, and a monolith. In some embodiments, the methods further comprise washing the oligo (dT) magnetic bead, the resin, or the monolith following capturing the mRNA. In some embodiments, the wash solution comprises a buffer selected from the group consisting of Tris, HEPES, NaPi, KCl, NaCl, Urea, Arginine, and EDTA.

In some embodiments, disclosed herein are methods of producing an mRNA molecule comprising contacting a composition comprising a DNA template with an in vitro transcription reaction system comprising an RNA polymerase and ribonucleotides under conditions sufficient for in vitro transcription to produce the mRNA molecule, wherein the contacting occurs for between about 120 minutes and about 260 minutes, wherein the contacting occurs at a temperature between 25° C. and 45° C., wherein the in vitro transcription reaction system further comprises a buffer and a magnesium ion, and wherein the in vitro transcription reaction system lacks a polyamine and a reducing agent.

### BRIEF DESCRIPTION OF THE DRAWINGS

FIG. 1 is an agarose gel image of plasmid dsDNA used as template in RCA reactions.

FIG. 2 is an agarose gel image of un-linearized RCA DNA without and with heat denaturation prior to RCA reaction.

FIG. 3 is an agarose gel image of RCA DNA with and without various purification steps included. Lane 1 is undigested, unpurified RCA DNA; Lane 2 is undigested and purified RCA DNA; Lane 3 RCA DNA linearized with SapI without purification before or after linearization (used in sequential single vessel one pot reactions); Lane 4 RCA DNA linearized with SapI without purification before linearization but has purification post linearization; Lane 5 RCA DNA purified before linearization with SapI (has buffer addition); Lane 6 RCA DNA purified before linearization with SapI (has buffer addition) and also has purification post linearization; Lane 7 is SapI digested pDNA with purification post-linearization; Lane 8 is SapI digested pDNA without purification

FIG. 4 is an agarose gel image of RCA reactions using RCA DNA from prior RCA reactions as the template. Shown are 3 sequential RCA reactions using the prior reaction as template for the next reaction (5 ng template). The initial reaction (Lanes 2 and 3) use pDNA as template. Lanes 2, 4, and 6 are uncut RCA DNA and lanes 3, 5, and 7 are restriction digested with SapI to demonstrate linearization.

FIG. 5 is an agarose gel image with RNA products from IVT reactions. RNA yield is indicated below each lane. Lane 1 shows sequential single vessel ‘one-pot’ RNA; Lane 2 shows RNA from RCA DNA template with purification post linearization (standard RCA process); Lanes 3-4 used undigested RCA DNA templates, without and with purification; Lanes 5 shows RNA using pDNA template with purification post linearization; Lane 6 shows RNA using pDNA template without purification post linearization (one pot pDNA)

FIG. 6 shows RNA integrity from Bioanalyzer fragment analysis of mRNA generated from pDNA or RCA DNA templates without and with oligo (dT)-selection. These relate to samples in lanes 1, 5, and 6 in FIG. 5. pDNA-CA09 indicates IVT using plasmid template; pDNA-CA09 one pot indicates IVT using plasmid template without purification after linearization; RCA-CA09 one pot indicates IVT in sequential single vessel one pot reaction.

FIG. 7 shows the impact of Mg and ATP+CTP+GTP+pUTP on RNA concentration.

FIG. 8 shows the impact of Mg and ATP+CTP+GTP+pUTP on RNA integrity.

FIG. 9 shows a DNase enzyme and calcium 2-factor interaction contour plot for RNA integrity after 60 min DNA Digestion.

FIG. 10 shows a magnesium and manganese 2-factor interaction contour plot for presence of the impurity residual DNA after 45 min DNA Digestion.

FIG. 11 shows an electropherogram depicting residual protein output.

FIG. 12 is a bar graph demonstrating that increasing feeding frequency improves process performance.

FIG. 13 is a bar graph depicting yield, integrity, and 5′-cap results from Experiment 2.

FIG. 14 is a bar graph depicting yield, integrity, and 5′-cap results from AMBR® 15 and AMBR® 250-scale bolus feed versus continuous feed using plasmid DNA (pDNA) and synthetic DNA (USTAT).

FIG. 15 is a combination bar graph and scatter plot depicting three processes demonstrating agitation impacts on the presence of the impurity residual DNA and yield, respectively.

FIG. 16 is a combination bar graph and scatter plot depicting three processes demonstrating power per volume impacts on the presence of the impurity residual DNA and yield, respectively.

FIG. 17 is a bar graph depicting two processes demonstrating pyrophosphatase impacts on the presence of the impurity residual DNA.

FIG. 18 is a bar graph depicting the effect of different template concentrations on yield in the batch IVT process.

FIG. 19 is a bar graph depicting varied incubation temperature and time in the batch IVT process.

FIG. 20 is a bar graph depicting varied incubation time and template concentrations on a long RNA molecule at different concentrations in the batch IVT process.

FIG. 21 is a bar graph depicting incubation temperatures lower than 37 C° at 2 or 3 hours incubation on a long RNA molecule in the batch IVT process.

FIG. 22 is a bar graph depicting the effect of each NTP on cap incorporation.

FIG. 23 is a bar graph depicting the effect of magnesium acetate titration.

FIG. 24 is a bar graph depicting varied cap analogue concentrations in a 0.4 mM GTP feed IVT process.

FIG. 25 is a bar graph depicting varied cap analogue concentrations in a 0.8 mM GTP feed IVT process.

FIG. 26 is a bar graph depicting varied cap analogue concentrations in a 1 mM GTP feed IVT process.

FIGS. 27A-B shows RCA DNA yield using plasmid template concentrations from 0.5-5 ng/mL RCA reaction (FIG. 27A) and an agarose gel of the resulting RCA DNA products using those template concentrations after linearization using BspQI (FIG. 27B).

FIGS. 28A-B show RCA DNA yield when the reaction temperature is varied from 27-30° C. (FIG. 28A) and an agarose gel of the RCA DNA products produced at those temperatures after linearization with BspQI (FIG. 28B).

FIG. 29 shows an agarose gel with RCA DNA products before and after digest with various concentrations of restriction enzyme BspQI. In all cases the prominent products are 5.2 kb as expected.

FIGS. 30A-B show results for RCA DNA products used as template for small-scale IVT reactions. Commercial RCA formulation was compared with optimized USTAT reaction to produce RCA DNA template (FIG. 30A) that was used to transcribe RNA in IVT reactions (FIG. 30B). Linearized and purified plasmid DNA was included as control for IVT. RNA integrity was measured by fragment analysis.

FIGS. 31A-B show results using USTAT RCA DNA versus linearized, purified plasmid DNA as template for IVT FB V1 at 250 mL scale. RNA yield and integrity results are shown in FIG. 31A. Integrity was measured using fragment analysis. The % capped mRNA was measured for products generated using plasmid DNA or RCA DNA templates and shown in FIG. 31B.

FIGS. 32A-D show analysis of next generation sequencing data from linearized plasmid control; linearized, unpurified plasmid; and USTAT RCA DNA. FIG. 32A shows similar coverage across the sequence in each sample and FIG. 32B shows high percentage of sequence without any insertion or deletions across samples. FIG. 32C shows sequence coverage throughout the transcription template and FIG. 32D shows no sequence variants observed in the sequence template.

FIG. 33 shows GFP protein expression in HEK293 cells over time after transfection with RNA transcribed from plasmid DNA control template or USTAT RCA DNA template. Either 0.5 ug or 0.05 ug GFP-encoding RNA was incubated in the transfection mixture.

FIGS. 34A-B show results for RCA DNA template in small scale IVT reactions. Commercial reaction buffer was compared to optimized USTAT reactions to produce RCA DNA with reaction yield shown in FIG. 34A. The DNA was used as template in IVT reactions (FIG. 34B). RNA integrity was measured using fragment analysis (FA).

FIG. 35 is a plot of IVT results for various sized transcripts using USTAT RCA DNA as template. USTAT RCA reactions were primed with either random primers or primase enzyme. Linearized plasmid template was included as a control. RNA integrity was measured using FA and yield by UV.

FIG. 36 is a plot of IVT results using assembled DNA template for USTAT. Assembled DNA templates were treated either with or without T5 exonuclease digestion prior to USTAT. Plasmid DNA template for USTAT was included as a control. RNA integrity was measured using FA.

FIG. 37 is a plot showing results of increasing Mg addition frequency during IVT reactions resulting in improved RNA quality.

FIG. 38 is a plot showing results of magnesium addition during an IVT reaction resulting in marked impacts to RNA quality attributes and yield.

FIG. 39 is a plot showing a comparison of BV1 and FB V2 across seven RNA constructs.

### DETAILED DESCRIPTION

mRNA technology has tremendous potential to treat diseases. To achieve that potential, high-quality mRNA for vaccine or therapeutic purposes is needed. In vitro transcription (IVT) can be carried out to synthesize RNA from a DNA template using one of the three bacteriophage SP6, T7 or T3 RNA polymerases. Traditionally, the RNA molecules are made using batch mode in vitro transcription where the reaction components are combined and then incubated for a few hours. Another approach to produce RNA molecules is by fed-batch in vitro transcription where any of the reaction components are added at certain time intervals.

Disclosed herein are batch and fed-batch methods for production of RNA molecules, e.g., mRNA, including modified mRNA molecules and/or self-amplifying RNA (saRNA), that are useful for producing clinical grade RNA, such as mRNA, of high purity and potency, consistently, reproducibly, and in compliance with current good manufacturing practices (cGMP). The methods use a DNA template in an in vitro transcription (IVT) reaction to generate the RNA molecule and can be applied to a wide variety of constructs with varying 5′ UTRS, coding sequence lengths, 3′ UTRs, and 3′ ends. In some aspects, the RNA molecule is purified via chromatographic methods, e.g., through use of an oligo (dT) substrate. In some aspects, enzymatic capping is used for 5′ capping of the RNA molecule.

Also disclosed herein are methods for various “one pot” reactions for production of RNA molecules, e.g., mRNA, including modified mRNA molecules, unmodified mRNA molecules, and/or saRNA, that are useful for producing clinical grade RNA, such as mRNA, of high purity and potency, consistently, reproducibly, and in compliance with current good manufacturing practices (cGMP). One or more steps of the methods (e.g., amplification, linearization, IVT, and/or capping) are performed in the same reaction vessel without intermediate nucleic acid purification processes between the step(s). In some aspects, all steps of the method steps (e.g., amplification, linearization, IVT, and/or capping) are performed in the same reaction vessel without intermediate nucleic acid purification processes between the steps.

### Certain Definitions

Throughout this application, the term “about” is used according to its plain and ordinary meaning in the area of cell and molecular biology to indicate that a value includes the inherent variation or standard deviation of error for the measurement or quantitation method being employed to determine the value. For example, in some aspects, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the measurement or quantitation.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The phrase “essentially all” is defined as “at least 95%”; if essentially all members of a group have a certain property, then at least 95% of members of the group have that property. In some instances, essentially all means equal to any one of, at least any one of, or between any two of 95, 96, 97, 98, 99, or 100% of members of the group have that property.

The compositions and methods for their use can “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that aspects described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one embodiment,” “an embodiment,” “a particular embodiment,” “a related embodiment,” “a certain embodiment,” “an additional embodiment,” “a further embodiment,” “some embodiments”, “one aspect,” “an aspect,” “a particular aspect,” “a related aspect,” “a certain aspect,” “an additional aspect,” “a further aspect,” “some aspects” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “inhibiting” or “reducing” or any variation of these terms includes any measurable decrease or complete inhibition to achieve a desired result. The terms “improve,” “promote,” or “increase” or any variation of these terms includes any measurable increase to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “DNA,” as used herein, means a nucleic acid molecule that includes deoxyribonucleotide residues (such as containing the nucleotide base(s) adenine (A), cytosine (C), guanine (G) and/or thymine (T)). For example, DNA can contain all, or a majority of, deoxyribonucleotide residues. As used herein, the term “deoxyribonucleotide” means a nucleotide lacking a hydroxyl group at the 2′ position of a β-D-ribofuranosyl group. Without any limitation, DNA can encompass double stranded DNA, antisense DNA, single stranded DNA, isolated DNA, synthetic DNA, DNA that is recombinantly produced, and modified DNA.

The term “RNA,” as used herein, means a nucleic acid molecule that includes ribonucleotide residues (such as containing the nucleotide base(s) adenine (A), cytosine (C), guanine (G) and/or uracil (U) or N-1-methylpseudouridine). For example, RNA can contain all, or a majority of, ribonucleotide residues. As used herein, the term “ribonucleotide” means a nucleotide with a hydroxyl group at the 2′ position of a β-D-ribofuranosyl group. In one aspect, RNA can be messenger RNA (mRNA) that relates to an RNA transcript which encodes a peptide or protein. As known to those of skill in the art, mRNA generally contains a 5′ untranslated region (5′-UTR), a polypeptide coding region, and a 3′ untranslated region (3′-UTR). Without any limitation, RNA can encompass double stranded RNA, antisense RNA, single stranded RNA, isolated RNA, synthetic RNA, RNA that is recombinantly produced, circular RNA, self-amplifying RNA (saRNA), and modified RNA (modRNA).

As contemplated herein, without any limitations, RNA can be used as a therapeutic modality to treat and/or prevent a number of conditions in mammals, including humans. Methods contemplated comprise administration of the RNA described herein to a mammal, such as a human. For example, in one aspect, such methods of use for RNA include an antigen-coding RNA vaccine to induce robust neutralizing antibodies and accompanying/concomitant T-cell response to achieve protective immunization with preferably minimal vaccine doses. The RNA administered is preferably in vitro transcribed RNA.

An “isolated RNA” is defined as an RNA molecule that can be recombinant or has been isolated from total genomic nucleic acid. A “modified RNA” or “modRNA” refers to an RNA molecule, e.g., an mRNA molecule, having at least one addition, deletion, substitution, and/or alteration of one or more nucleotides as compared to naturally occurring RNA. Such alterations can refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide can replace one or more uridine and/or cytidine nucleotides. For example, these replacements can occur for every instance of uridine and/or cytidine in the RNA sequence, or can occur for only select uridine and/or cytidine nucleotides. Such alterations to the standard nucleotides in RNA can include non-standard nucleotides, such as chemically synthesized nucleotides or deoxynucleotides. For example, at least one uridine nucleotide can be replaced with 1-methylpseudouridine in an RNA sequence. Other such altered nucleotides are known to those of skill in the art. Such altered RNAs are considered analogs of naturally-occurring RNA. In some aspects, the RNA is produced by in vitro transcription using a DNA template, where DNA refers to a nucleic acid that contains deoxyribonucleotides. In some aspects, the RNA can be replicon RNA (replicon), in particular self-replicating RNA, or self-amplifying RNA (saRNA).

As used herein, a “protein,” “polypeptide,” or “peptide” refers to a molecule comprising at least two amino acid residues. As used herein, the term “wild-type” or “native” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. Where a protein is specifically mentioned herein, it is in general a reference to a native (wild-type) or recombinant (modified) protein. The protein may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., an antigen or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The term “isolated” can refer to a nucleic acid or polypeptide that is substantially free of cellular material, bacterial material, viral material, or culture medium (e.g., when produced by recombinant DNA techniques) of their source of origin, or chemical precursors or other chemicals (e.g., when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state and/or that is altered or removed from the natural state through human intervention. For example, a DNA naturally present in a living animal is not “isolated,” but a synthetic DNA, or a DNA partially or completely separated from the coexisting materials of its natural state is “isolated.” An isolated nucleic acid can exist in substantially purified form, or can exist in a non-native environment such as, for example, a cell into which the nucleic acid has been delivered.

All patents, published patent applications, other publications, and databases referred to herein are incorporated by reference in their entirety with respect to the related technology.

### DNA Template

In some aspects, the method for producing an RNA molecule, e.g., mRNA, includes providing a sample that includes a linear DNA template. The DNA template includes a sequence coding for a gene of interest that encodes, e.g., a peptide or polypeptide of interest. In some aspects, the DNA template includes an RNA polymerase promoter sequence operably linked to the sequence coding for a gene of interest. In some aspects, the DNA template includes an RNA polymerase promoter sequence operably linked to the respective RNA polymerase gene sequence, which is operably linked to a subgenomic promoter, which is operably linked to a sequence coding for a gene of interest. For example, in some preferred aspects, the DNA template includes an RNA-dependent RNA polymerase (RdRp) promoter sequence operably linked to the RdRp gene sequence, which is operably linked to a subgenomic promoter, which is operably linked to a sequence coding for a gene of interest. In some aspects, a DNA template lacks a plasmid backbone.

In some aspects, the linear DNA template is a linearized plasmid DNA used as the template for in vitro transcription. In some aspects, cells, e.g., bacterial cells, e.g., E. coli, e.g., DH10B cells, are transfected with the plasmid DNA template. The transfected cells are cultured to replicate the plasmid DNA which is then isolated and purified. In some aspects, the linear DNA template is synthesized in a cell-free environment. In some aspects, the linear DNA template is synthesized by rolling circle amplification (RCA). In some aspects, RCA includes an amplification target circle (ATC) that forms a template on which new DNA is made, thereby extending the initial sequence as a continuous sequence of repeated sequences complementary to the circle but generating only about several thousand copies per hour. In some aspects, the linear DNA template is provided by exponential RCA, including hyperbranched RCA (also termed ramification amplification).

In other aspects, the linear DNA template is provided by a cell-free process for synthesizing DNA that includes contacting a DNA template with at least one polymerase in the presence of nucleotides to form a reaction mixture, wherein the DNA template is amplified by strand displacement replication, and wherein further nucleotides are supplied to the reaction mixture continuously or at intervals during the process.

In some aspects, the DNA template also includes an RNA polymerase promoter sequence, e.g., a T7 promoter, located 5′ to and operably linked to the gene of interest. In some aspects, the DNA template includes an RNA polymerase promoter sequence operably linked to the respective RNA polymerase gene sequence, which is operably linked to a subgenomic promoter, which is operably linked to a sequence coding for a gene of interest. In some preferred aspects, the DNA template includes an RNA-dependent RNA polymerase (RdRp) promoter sequence, located 5′ to and operably linked to a subgenomic promoter, which is operably linked to a sequence coding for a gene of interest. As used herein, the phrase “operably linked” refers to a functional connection between two or more molecules, constructs, transcripts, entities, moieties or the like. For example, a gene of interest operably linked to an RNA polymerase promoter allows transcription of the gene of interest. Any RNA polymerase or variants thereof may be used in the methods described here. The RNA polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids.

As used herein, “gene of interest” refers to a polynucleotide which encodes a polypeptide or protein of interest. Depending on the context, the gene of interest refers to a deoxyribonucleic acid, e.g., a gene of interest in a DNA template which may be transcribed to an RNA molecule, or a ribonucleic acid, e.g., a gene of interest in an RNA molecule which may be translated to produce the encoded polypeptide of interest in vitro, in vivo, in situ or ex vivo. As described in more detail below, a polypeptide of interest includes but is not limited to, biologics, antibodies, vaccines, therapeutic proteins or peptides, etc.

### Flanking Regions: Untranslated Regions (UTRs)

In some aspects, the method for producing an RNA molecule includes (a) providing a sample having a linear DNA template, the DNA template includes an RNA polymerase promoter sequence operably linked to a sequence coding for a gene of interest and a 5′ untranslated region (UTR) and/or a 3′ UTR. In some aspects, the DNA template includes an RNA polymerase promoter sequence operably linked to the respective RNA polymerase gene sequence, which is operably linked to a subgenomic promoter, which is operably linked to a sequence coding for a gene of interest. For example, in some aspects, the method for producing an RNA molecule includes (a) providing a sample having a linear DNA template, the DNA template includes an RNA-dependent RNA polymerase (RdRp) promoter sequence, located 5′ to and operably linked to a subgenomic promoter, which is operably linked to a sequence coding for a gene of interest and a 5′ untranslated region (UTR) and/or a 3′ UTR.

The DNA template and RNA molecule may include UTRs. Untranslated regions (UTRs) of a gene are transcribed but not translated. The 5′ UTR starts at the transcription start site and continues to the start codon but does not include the start codon; whereas, the 3′ UTR starts immediately following the stop codon and continues until the transcriptional termination signal. The regulatory features of a UTR may be incorporated into the polynucleotides, primary constructs and/or mRNA of the present invention to enhance the stability of the molecule. The specific features may also be incorporated to ensure controlled down-regulation of the transcript in case they are misdirected to undesired organs sites.

Natural 5′ UTRs bear features which play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTR also have been known to form secondary structures which are involved in elongation factor binding. By engineering the features typically found in abundantly expressed genes of specific target organs, one may enhance the stability and protein production of the polynucleotides, primary constructs. For example, use of 5′ UTR from other tissue-specific mRNA to improve expression in that tissue is possible for muscle (MyoD, Myosin, Myoglobin, Myogenin, Herculin), for endothelial cells (Tie-1, CD36), etc.

Other non-UTR sequences may be incorporated into the 5′ (or 3′ UTR) UTRs. For example, introns or portions of introns sequences may be incorporated into the flanking regions of the polynucleotides, primary constructs or mRNA described here. Incorporation of intronic sequences may increase protein production as well as mRNA levels. Cap-dependent translation involves recruitment of the pre-initiation complex (PIC) to the 5′ end of an mRNA followed by scanning to find an AUG initiation codon in an optimum sequence context. AUG recognition promotes scanning cessation, release of most initiation factors, and recruitment of the large ribosomal subunit to initiate elongation. Efficient recognition of an initiation codon depends on its surrounding sequence. In some preferred aspects, the DNA template includes an initiation codon GGG. In some preferred aspects, the DNA template includes an initiation codon AGA. In some aspects, the sequence CRCCaugG (R=purine, A or G) may provide optimal context for AUG recognition in eukaryotes.

3′ UTRs are known to have stretches of Adenosines and Uridines embedded in them. These AU rich signatures are particularly prevalent in genes with high rates of turnover. Based on their sequence features and functional properties, the AU rich elements (AREs) may be separated into three classes: Class I AREs include several dispersed copies of an AUUUA motif within U-rich regions. C-Myc and MyoD include class I AREs. Class II AREs possess two or more overlapping UUAUUUA(U/A)(U/A) nonamers. Molecules including this type of AREs include GM-CSF and TNF-alpha. Class III ARES are less well defined. These U rich regions do not include an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules may lead to HuR binding and thus, stabilization of the message in vivo. Introduction, removal or modification of 3′ UTR AREs may be used to modulate the stability of polynucleotides, and primary constructs.

When engineering specific polynucleotides, and/or primary constructs, one or more copies of an ARE may be introduced to make polynucleotides, and/or primary constructs less stable and thereby curtail translation and decrease production of the resultant protein. Likewise, AREs may be identified and removed or mutated to increase the intracellular stability and thus increase translation and production of the resultant protein. Transfection experiments may be conducted in relevant cell lines, using polynucleotides, and/or primary constructs and protein production may be assayed at various time points post-transfection. For example, cells may be transfected with different ARE-engineering molecules and by using an ELISA kit to the relevant protein and assaying protein produced at 6 hour, 12 hour, 24 hour, 48 hour, and 7 days post-transfection.

### Poly(A) Tail

In some aspects, the methods for producing an RNA molecule disclosed herein include providing a sample having a DNA template, the DNA template having an RNA polymerase promoter sequence operably linked to a sequence coding for a gene of interest and a poly(A) tail sequence of 20-100 nucleotides. The poly(A) tail can prevent degradation of the RNA molecule in a cell. Accordingly, in some aspects, the plasmid DNA template includes a sequence coding for a poly(A) tail located 3′ to the gene of interest. As used herein, “poly(A) tail” refers to a chain of adenine nucleotides. In some aspects, the poly(A) tail includes 5-300 adenine nucleotides in length, e.g., at least, at most, or about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 adenine nucleotides in length, or any range or value derivable therein. In preferred aspects, the DNA template includes a poly(A) tail that includes about 40 adenines. In preferred aspects, the DNA template includes a poly(A) tail that includes about 80 adenines. In some aspects, the poly(A) tail is encoded in the DNA template. In other aspects, the poly(A) tail is added to the RNA molecule by enzymatic treatment with a Poly(A) polymerase. In some aspects, the RNA molecule does not include a poly(A) tail.

In some aspects, immediately downstream of the poly(A) tail coding sequence on the plasmid DNA template is a recognition site for a restriction endonuclease to linearize the plasmid. Linearization of the plasmid can mitigate transcriptional readthrough.

In some aspects, following linearization, the plasmid DNA template is filtered into an appropriate solvent, e.g., water, HEPES, and EDTA. In a preferred aspect, the solvent includes 10 mM HEPES, 0.1 mM EDTA, and the like. Filtration occurs via, e.g., ultrafiltration, diafiltration, or, e.g., tangential flow ultrafiltration/diafiltration.

The linear DNA template may be purified before use as a template for in vitro transcription. For example, the linear DNA template may be purified chromatographically or by ethanol precipitation.

### In Vitro Transcription

In vitro transcription (IVT) refers to a procedure that allows for DNA-directed synthesis of RNA molecules of any sequence, ranging in size from short oligonucleotides to several kilobases. In some aspects, in vitro transcription involves engineering of a DNA template to include a bacteriophage promoter sequence (e.g., from the T7 coliphage) upstream of the sequence of interest followed by transcription using the corresponding RNA polymerase. In some aspects, the resulting RNA molecules are subsequently modified (e.g., by capping, splicing, the addition of a poly(A) tail, etc.).

The methods described herein for producing an RNA molecule, e.g., mRNA, includes contacting a DNA template with an in vitro transcription (IVT) reaction system. In some aspects, the IVT reaction system includes an RNA polymerase and ribonucleotides, which may be natural and/or modified ribonucleotides. In some aspects, the IVT reaction system includes a transcription buffer, nucleotide triphosphates (NTPs), an RNase inhibitor and an RNA polymerase. The NTPs may be selected from, but are not limited to, those described herein including natural and unnatural (modified, such as, for example, N-1-methylpseudouridine triphosphate) NTPs.

### RNA Polymerase

In some aspects, the RNA polymerase used to generate the mRNA transcript may also be referred to as a “DNA-dependent RNA polymerase” which transcribes DNA into RNA molecules. Exemplary RNA polymerases include bacteriophage T7, T3, Syn5, and SP6 RNA polymerases, or variants thereof (including thermostable/thermophilic variants), which may be used to transcribe the mRNA or self-amplifying RNA from a DNA template. RNA polymerases represent the primary machinery that drives transcription. RNA polymerases have been isolated and purified sufficiently that they are useful for producing RNA in vitro. In some aspects, the RNA polymerase is a T7 RNA polymerase, which refers to a monomeric T7 bacteriophage-encoded DNA directed RNA polymerase that catalyzes the formation of RNA in the 5′ to 3′ direction. The wild-type T7 RNA polymerase includes 883 amino acids. It is homologous to T3 RNA polymerase and somewhat homologous to SP6 RNA polymerase.

In some aspects, the RNA polymerase includes an engineered T7 RNA polymerase variant, such as a variant that allows for selective incorporation of the m7G(5′)ppp(5′) m7G cap analog over GTP at the initiation of in vitro transcription. For example, in some aspects, the RNA polymerase has been modified to preferentially accept a cap (also referred to as an RNA cap, an RNA 7-methylguanosine cap or an RNA m7G cap) or cap analog (e.g., the “Anti Reverse Cap Analog” (3′-O-Me-m7G (5′)ppp(5′) G; “ARCA”), or a methylated cap analog with one or more nucleotides at the transcription initiation site (e.g., m7G(5′)ppp(5′) N, wherein N is any nucleotide) to begin transcription during transcription initiation. The 5′ cap is an altered nucleotide on the 5′ end of some eukaryotic primary transcripts such as precursor messenger RNA. The typical cap structure includes a 7-methylguanosine (m7G) linked to the first nucleotide of the transcript via a 5′-5′ triphosphate bridge. Cap analogs may include, for example, one, two or more methyl (or other substitution) groups at specific positions. Cap molecules may be added either upfront in the IVT reaction or after the synthesis of mRNA, by enzymatic capping. Cap molecules added upfront in the IVT reaction can make the mRNA production more straightforward.

### Ribonucleotides

In the methods described herein, the IVT reaction system includes nucleotides (for example, non-modified ribonucleotide triphosphates or modified ribonucleotide triphosphates). The nucleotides may be selected from any one of natural nucleotides, e.g., A, G, C, and U ribonucleotides; modified nucleotides (such as, for example, N-1-methylpseudouridine triphosphate); or a combination thereof. In some aspects, the ribonucleotides are Tris buffered, such as a 100 mM aqueous solution of ribonucleotide titrated to pH 7.3-7.5 with Tris base. In some aspects, the ribonucleotides are in sodium salt.

Modified nucleobases which may be incorporated into modified nucleosides and nucleotides and be present in the RNA molecules generated by the IVT reaction system include, for example, m5C (5-methylcytidine), m5U (5-methyluridine), m6A (N6-methyladenosine), s2U (2-thiouridine), Um (2′-O-methyluridine), mlA (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl) adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar (p) (2′-O-ribosyladenosine (phosphate)); I (inosine); mil (1-methylinosine); m′lm (1,2′-O-dimethylinosine); m3C (3-methylcytidine); Cm (2T-O-methylcytidine); s2C (2-thiocytidine); ac4C (N4-acetylcytidine); £5C (5-fonnylcytidine); m5Cm (5,2-O-dimethylcytidine); ac4Cm (N4acetyl2TOmethylcytidine); k2C (lysidine); mlG (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr (p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl) uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl) uridine)); mchm5U (5-(carboxyhydroxymethyl) uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm 5Um (5-carboxymethy 1 aminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6, T-O-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); mlGm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-C6)-alkyluracil, 5-methyluracil, 5-(C2-Ce)-alkenyluracil, 5-(C2-Ce)-alkynyluracil, 5-(hydroxymethyl) uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Additional exemplary modified nucleotides include any one of N-1-methylpseudouridine; pseudouridine, N6-methyladenosine, 5-methylcytidine, and 5-methyluridine.

In some aspects, the RNA molecule may include phosphoramidate, phosphorothioate, and/or methylphosphonate linkages.

In some aspects, the RNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine. In other aspects, the RNA may include a 5′ cap comprising a 7′-methylguanosine, and the first 1, 2 or 3 5′ ribonucleotides may be methylated at the 2′ position of the ribose.

### Exemplary In Vitro Transcription Reaction Systems

In some aspects, the in vitro transcription reaction system includes the following: an RNA polymerase, e.g., a T7 RNA polymerase, DNA template; nucleotide triphosphates (NTPs); magnesium; and a buffer such as, e.g., HEPES or Tris (or both HEPES and Tris). In some aspects, the in vitro transcription reaction system includes the RNA

polymerase, e.g., a T7 RNA polymerase, at a final concentration of 1000-44000 U/mL, e.g., at least, at most, or about 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200, 7250, 7300, 7350, 7400, 7450, 7500, 7550, 7600, 7650, 7700, 7750, 7800, 7850, 7900, 7950, 8000, 8050, 8100, 8150, 8200, 8250, 8300, 8350, 8400, 8450, 8500, 8550, 8600, 8650, 8700, 8750, 8800, 8850, 8900, 8950, 9000, 9050, 9100, 9150, 9200, 9250, 9300, 9350, 9400, 9450, 9500, 9550, 9600, 9650, 9700, 9750, 9800, 9850, 9900, 9950, 10000, 10050, 10100, 10150, 10200, 10250, 10300, 10350, 10400, 10450, 10500, 10550, 10600, 10650, 10700, 10750, 10800, 10850, 10900, 10950, 11000, 11050, 11100, 11150, 11200, 11250, 11300, 11350, 11400, 11450, 11500, 11550, 11600, 11650, 11700, 11750, 11800, 11850, 11900, 11950, 12000, 12500, 13000, 13500, 14000, 14500, 15000, 15500, 16000, 16500, 17000, 17500, 18000, 18500, 19000, 19500, 20000, 20500, 21000, 21500, 22000, 22500, 23000, 23500, 24000, 24500, 25000, 25500, 26000, 26500, 27000, 27500, 28000, 28500, 29000, 29500, 30000, 30500, 31000, 31500, 32000, 32500, 33000, 33500, 34000, 34500, 35000, 35500, 36000, 36500, 37000, 37500, 38000, 38500, 39000, 39500, 40000, 40500, 41000, 41500, 42000, 42500, 43000, 43500, or 44000 U/mL, or any range or value derivable therein. In some aspects, the in vitro transcription reaction system includes a T7 RNA polymerase at either 8U/uL, 10U/uL, 13U/uL, or 15U/uL. In some aspects, the in vitro transcription reaction system includes an RNA polymerase, e.g., a T7 RNA polymerase, at a final concentration of 7000 U/mL. In some aspects, the in vitro transcription reaction system includes an RNA polymerase, e.g., a T7 RNA polymerase, at a final concentration of 8000 U/mL. In some aspects, the in vitro transcription reaction system includes an RNA polymerase, e.g., a T7 RNA polymerase, at a final concentration of 14000 U/mL. In some aspects, the in vitro transcription reaction system includes an RNA polymerase, e.g., a T7 RNA polymerase, at a final concentration of 17000 U/mL. In some aspects, the in vitro transcription reaction system includes an RNA polymerase, e.g., a T7 RNA polymerase, at a final concentration of 25000 U/mL. In some aspects, the in vitro transcription reaction system includes an RNA polymerase, e.g., a T7 RNA polymerase, at a final concentration of 40000 U/mL.

In some aspects, the in vitro transcription reaction system includes the DNA template at a final concentration of, e.g., at least, at most, or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 nM, or any range or value derivable therein. In some aspects, the in vitro transcription reaction system includes the DNA template at a final concentration of 40 nM. In some aspects, the in vitro transcription reaction system includes the DNA template at a final concentration of 144 nM. In some aspects, the in vitro transcription reaction system includes the DNA template at a final concentration of 5-24 nM DNA, e.g., at least, at most, or about 5, 6, 7, 8, 9, 10, 11, 12, 13, 14, 15, 16, 17, 18, 19, 20, 21, 22, 23, or 24 nM, or any range or value derivable therein. In some aspects, the in vitro transcription reaction system includes the DNA template at a final concentration of 36 to 144 nM DNA, e.g., at least, at most, or about 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, or 144 nM, or any range or valuable derivable therein. In some aspects, the in vitro transcription reaction system includes the DNA template at a final concentration of, e.g., at least, at most, or about 0.01, 0.02, 0.025, 0.03, 0.04, 0.05, 0.06, 0.07, 0.075, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50 mg/mL, or any range or value derivable therein. In some aspects, the in vitro transcription reaction system includes the DNA template at a final concentration of 0.025 mg/mL. In some aspects, the in vitro transcription reaction system includes the DNA template at a final concentration of 0.05 mg/mL. In some aspects, in vitro transcription reaction system includes the DNA template at a final concentration of 0.075 mg/mL. In some aspects, the in vitro transcription reaction system includes the DNA template at a final concentration of 0.1 mg/mL.

In some aspects, the in vitro transcription reaction system includes each nucleotide triphosphate (NTP) at a final concentration of, e.g., at least, at most, or about 0.4, 0.8, 1.0, 1.25, 2, 3, 4, 5, 6, 7, 8, 9, 10, 11, 12, 13, 14, 15, 16, 17, 18, 19, 20, 21, 22, 23, 24, 25, or 26 mM, or any range or value derivable therein. In some aspects, the in vitro transcription system includes each nucleotide triphosphate (NTP) at a starting concentration of, e.g., at least, at most, or about 0.4, 0.8, 1.0, 1.25, 2, 3, 4, 5, 6, 7, 8, 9, 10, 11, 12, 13, 14, 15, 16, 17, 18, 19, 20, 21, 22, 23, 24, 25, or 26 mM. In some aspects, the in vitro transcription reaction system includes the nucleotide triphosphates (NTPs) at a final concentration of about 8 mM each. In some aspects, the in vitro transcription reaction system includes the nucleotide triphosphate ATP at a final concentration of, e.g., at least, at most, or about 2, 3, 4, 5, 6, 7, 8, 9, 10, 11, 12, 13, 14, 15, 16, 17, 18, 19, 20, 21, 22, 23, 24, 25, or 26 mM, or any range or value derivable therein. In some aspects, the in vitro transcription reaction system includes the nucleotide triphosphate CTP at a final concentration of, e.g., at least, at most, or about 2, 3, 4, 5, 6, 7, 8, 9, 10, 11, 12, 13, 14, 15, 16, 17, 18, 19, 20, 21, 22, 23, 24, 25, or 26 mM, or any range or value derivable therein. In some aspects, the in vitro transcription reaction system includes the nucleotide triphosphate GTP at a final concentration of, e.g., at least, at most, or about 2, 3, 4, 5, 6, 7, 8, 9, 10, 11, 12, 13, 14, 15, 16, 17, 18, 19, 20, 21, 22, 23, 24, 25, or 26 mM, or any range or value derivable therein. In some aspects, the in vitro transcription reaction system includes the UTP or nucleotide triphosphate modified UTP at a final concentration of, e.g., at least, at most, or about 2, 3, 4, 5, 6, 7, 8, 9, 10, 11, 12, 13, 14, 15, 16, 17, 18, 19, 20, 21, 22, 23, 24, 25, or 26 mM, or any range or value derivable therein. In certain aspects the concentration of NTPs in the reaction is 0.4 mM, 0.8 mM, 1 mM, 1.25 mM, 3. mM, 5 mM, 6 mM, 7 mM, 7.5 mM, 8 mM, 8.5 mM, 9 mM, 9.5 mM, or 10 mM.

In some aspects, the IVT reaction system includes magnesium ion, for example, as a magnesium salt, such as any one of magnesium chloride and magnesium acetate. In some aspects, the in vitro transcription reaction system includes the magnesium at a final concentration of, e.g., at least, at most, or about 12, 13, 14, 15, 16, 16.5, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, or 220 mM, or any range or value derivable therein. In some aspects, the in vitro transcription reaction system includes magnesium acetate at 30 mM. In some aspects, the in vitro transcription reaction system includes magnesium acetate at 40 mM. In some aspects, the in vitro transcription reaction system includes magnesium acetate at 16.5 mM. In some aspects, the in vitro transcription reaction system includes magnesium acetate at 33 mM. In some aspects, the in vitro transcription reaction system includes magnesium acetate at 36 mM. In some aspects, the in vitro transcription reaction system includes magnesium acetate at 50 mM. In some aspects, the in vitro transcription reaction system includes magnesium acetate at 110 mM. In some aspects, the Mg:NTP ratio can be maintained at a ratio of, e.g., at least, at most, or about, 0, 0.8, 0.9, 1.0, 1.1, 1.2, 1.3, 1.4, 1.5, 1.6, 1.7, 1.8, 1.9, 2.0, 2.1, or 2.2 mM Mg/mM NTP, or any range or value derivable therein. In some aspects, one or more reaction components are added during in vitro transcription by occasional bolus feeds, semi-continuous feeds, or continuous feeds. Bolus feeds can be delivered at intervals of, e.g., at least, at most, or about, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 minutes, or any range or value derivable therein. These components can include, but are not limited to, one or more NTPs and a cation such as magnesium. These components can be combined into a single feed, or they can be delivered separately in the form of multiple feeds. In some aspects, a continuous feed of at least 1 NTP can be delivered at flow rates of, e.g, at least, at most, or about 0, 0.5, 1, 1.5, 2, 2.5, 3, 3.5, or 4 mL/L/min. In some aspects, a continuous feed of a cation such as magnesium can be delivered at concentrations of, e.g., at least, at most, or about 0, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.2, 0.25, 0.3, 0.35, 0.4, 0.45, 0.5, 0.55, 0.6, 0.65, 0.7, 0.75, 0.8, 0.85, 0.9, 0.95, or 1.0 mM/min, or any range or value derivable therein

In some aspects, the in vitro transcription (IVT) reaction system includes a buffer. Exemplary buffers for the IVT reaction system may include Tris and/or HEPES. In some aspects, the in vitro transcription reaction system includes the buffer at a pH of, e.g., at least, at most, or about 7, 7.1, 7.2, 7.3, 7.4, 7.5, 7.6, 7.7, 7.8, 7.9, 8, 8.1, 8.2, 8.3, 8.4, or 8.5, or any range or value derivable therein. In some aspects, the buffer is Tris-HCl, pH 8.0. In some aspects, the in vitro transcription reaction system includes 40 mM Tris HCl, pH 8.0. Alternative buffers for the IVT reaction system include 40 mM Tris pH 7.5, 80 mM HEPES. In some aspects, the in vitro transcription reaction system does not include HEPES. In some aspects, the in vitro transcription reaction system includes HEPES and Tris. In some aspects, the buffer does not comprise dithiothreitol (DTT).

In some aspects, an RNase inhibitor is included in the in vitro transcription reaction system. The RNase inhibitor may reduce RNase-induced degradation during the transcription reaction. For example, murine RNase inhibitor may be utilized at a final concentration of 40, 50, 60, 70, 80, 90, 100, 150, 200, 250, 300, 350, 400, 450, 500, 550, 600, 650, 700, 750, 800, 850, 900, 950, 1000, 1050, 1100, 1150, or 1200 U/mL. In some aspects, the in vitro transcription reaction system comprises RNase inhibitor at a final concentration of 100 U/mL. In some aspects, the in vitro transcription reaction system comprises RNase inhibitor at a final concentration of 1000 U/mL.

In some aspects, a pyrophosphatase is included in the in vitro transcription reaction system. The pyrophosphatase may cleave the inorganic pyrophosphate generated following each nucleotide incorporation into two units of inorganic phosphate, which may reduce the likelihood of magnesium co-precipitating with pyrophosphate to form magnesium pyrophosphate. Pyrophosphatase in certain aspects may be diluted in pyrophosphatase buffer and present in the reaction at concentrations of 0.01 mU/uL, 0.02 mU/uL, 0.05 mU/uL, 0.08 mU/uL, 0.1 mU/uL, 0.2 mU/uL, 0.8 mU/uL, or 2 mU/uL. In some aspects, the in vitro transcription reaction system includes an inorganic pyrophosphatase at a final concentration of 0.25 U/mL. In some aspects, the in vitro transcription reaction system includes an inorganic pyrophosphatase at a final concentration of 0.5 U/mL. In some aspects, the in vitro transcription reaction system includes an inorganic pyrophosphatase at a final concentration of 1 U/mL. In some aspects, the in vitro transcription reaction system includes an inorganic pyrophosphatase at a final concentration of 2 U/mL. In some aspects, the in vitro transcription reaction system includes an inorganic pyrophosphatase at a final concentration of 3 U/mL. In some aspects, the in vitro transcription reaction system includes an inorganic pyrophosphatase at a final concentration of 6 U/mL.

In some aspects, the in vitro transcription reaction system includes a polyamine. Exemplary polyamines include spermine, putrescene, and spermidine. In some aspects 1 mM spermidine is included. In some aspects 2.0 mM spermidine is included. In some aspects 2.15 mM spermidine is included. In some aspects, the in vitro transcription reaction system lacks a polyamine. In some aspects, the in vitro transcription reaction system lacks spermidine.

In some aspects, the IVT reaction system includes a reducing reagent, such as, for example, DTT (dithiothreitol), e.g., at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mM, or any range or value derivable therein. In some aspects, the reducing agent is selected from the group consisting of dithiothreitol (DTT), dithioerythritol (DTE), Tris(2-carboxyethyl) phosphine (TCEP) and beta-mercaptoethanol. In some aspects, the IVT reaction system includes 1 mM DTT. In some aspects, the IVT reaction system includes 5 mM DTT. In some aspects, the IVT reaction system includes 10 mM DTT. In some aspects, the IVT reaction system includes 20 mM DTT. In some aspects, the IVT reaction ssytem lacks a reducing agent. In some aspects, the IVT reaction system does not contain DTT. In some aspects, the IVT reaction system does not contain added DTT beyond the protective amount of DTT present in the T7 RNA polymerase storage solution.

In some aspects, the in vitro transcription reaction proceeds, for example, at about 37° C. for about 4 hours or about 240 minutes, e.g., at least, at most, or about 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, or 240 minutes, or any range or value derivable therein. In some preferred aspects, the in vitro transcription reaction proceeds, for example, at less than 50° C. for less than 4 hours, such as for example, at least, at most, or about 50° C., 49° C., 48° C., 47° C., 46° C., 45° C., 44° C., 43° C., 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., or about 20° C., for at least, at most, or about 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, or 240 minutes, or any range or value derivable therein. In some aspects, the in vitro transcription reaction proceeds at about 36° C. for about 120 minutes. In some aspects, the in vitro transcription reaction proceeds at about 36° C. for about 150 minutes. In some aspects, the in vitro transcription reaction proceeds at about 37° C. for about 120 minutes. In some aspects, the in vitro transcription reaction proceeds at about 37° C. for about 150 minutes.

In some aspects, the in vitro transcription reaction proceeds at about 37° C., at greater than 120 minutes and less than 360 minutes, preferably greater than 120 minutes and less than 300 minutes, more preferably greater than 120 minutes and less than 260 minutes. In some preferred aspects, the in vitro transcription reaction proceeds at about 37° C. for about 150 minutes.

As disclosed herein, a reaction system proceeding at about 37° C. for less than 4 hours results in high yields of highly pure RNA. In some aspects, yields per in vitro transcription reaction may be at least 0.3 mg of RNA per mL starting volume of IVT reaction to about 20 mg of RNA per mL starting volume of IVT reaction. For example, in some aspects, the total yield of RNA molecule may be at least, at most, or about 0.3 mg RNA/mL, 0.4 mg RNA/mL, 0.5 mg RNA/mL, 0.6 mg RNA/mL, 0.7 mg RNA/mL, 0.8 mg RNA/mL, 0.9 mg RNA/mL, 1.0 mg RNA/mL, 2 mg RNA/mL, 3 mg RNA/mL, 4 mg RNA/mL, preferably at least 5 mg RNA/mL, 6 mg RNA/mL, 7 mg RNA/mL, 8 mg RNA/mL, 9 mg RNA/mL, 10 mg RNA/mL, 11 mg RNA/mL, 12 mg RNA/mL, 13 mg RNA/mL, 14 mg RNA/mL, 15 mg RNA/mL, 16 mg RNA/mL, 17 mg RNA/mL, 18 mg RNA/mL, 19 mg RNA/mL, or 20 mg RNA/mL starting volume of IVT reaction, or any range or value derivable therein. In preferred aspects, the total yield per in vitro transcription reaction of RNA molecule produced having at least 90% of the intended full length transcript may be at least 2 mg RNA/mL, 3 mg RNA/mL, 4 mg RNA/mL, preferably at least 5 mg RNA/mL, 6 mg RNA/mL, 7 mg RNA/mL, 8 mg RNA/mL, 9 mg RNA/mL, 10 mg RNA/mL, 11 mg RNA/mL, 12 mg RNA/mL, 13 mg RNA/mL, 14 mg RNA/mL, 15 mg RNA/mL, 16 mg RNA/mL, 17 mg RNA/mL, 18 mg RNA/mL, 19 mg RNA/mL, or 20 mg RNA/mL starting volume of IVT reaction. In some aspects, the total yield per in vitro transcription reaction of RNA molecule produced having at least 90% of the intended full length transcript is at least 17 mg RNA/mL starting volume of IVT reaction.

In some aspects, following an IVT reaction using a DNA template and an RNA polymerase as described here, a first composition that includes an uncapped RNA molecule is produced. In some aspects, the RNA molecule includes the coding sequence for a gene of interest and a poly(A) tail. As used herein, the RNA molecule includes an mRNA. The RNA molecule may include modifications, such as, modified nucleotides. As used herein, an “RNA molecule” produced by in vitro transcription may be referred to as an “RNA transcript.” An “RNA molecule” and an “RNA transcript” may encompass any one of modified mRNA “modRNA,” unmodified mRNA, and self-amplifying RNA (saRNA). In preferred aspects, the RNA molecule is a saRNA.

In some aspects, the methods of producing RNA molecules by contacting the DNA sample with the in vitro transcription reaction system described herein produces a first composition having an uncapped RNA molecule. In some aspects, at least 30% of the RNA molecules in the first composition includes uncapped RNA molecules. In some aspects, the first composition includes at least, at most, or about 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range or value derivable therein, uncapped RNA molecules.

### RNA Molecule

The RNA molecule produced by the methods described herein may be a non-coding and/or a coding RNA. A non-coding RNA (ncRNA) molecule includes a functional RNA molecule that is not translated into a peptide or polypeptide. Non-coding RNA molecules may include highly abundant and functionally important RNAs such as transfer RNA (tRNA) and ribosomal RNA (rRNA), as well as RNAs such as snoRNAs, microRNAs, siRNAs, snRNAs, guideRNAs, circularRNAs, exRNAs, and piRNAs and long ncRNAs. In a preferred aspect, the RNA molecule is an mRNA molecule that includes a modified nucleotide (herein referred to as a “modified RNA molecule” or “modified mRNA molecule”). In some preferred aspects, the RNA molecule is a self-amplifying RNA molecule.

Coding RNA includes a functional RNA molecule that may be translated into a peptide or polypeptide. In some aspects, the coding RNA molecule includes at least one open reading frame coding for at least one peptide or polypeptide. The coding RNA molecule may include one (monocistronic), two (bicistronic) or more (multicistronic) open reading frames (ORFs). The coding RNA molecule may be a messenger RNA (mRNA) molecule, viral RNA molecule or self-amplifying RNA molecule (saRNA, also referred to as a replicon). Preferably, the RNA molecule is an mRNA.

The RNA molecule may encode more than one protein, e.g., two, three, four, five, ten or more polypeptides. Alternatively, or in addition, one RNA molecule may also encode more than one antigen, e.g., a bicistronic, or tricistronic RNA molecule that encodes different or identical antigens.

The sequence of the RNA molecule may be codon optimized or de-optimized for expression in a desired host, such as a human cell.

The sequence of the RNA molecule may be modified if desired, for example to increase the efficacy of expression or replication of the RNA, or to provide additional stability or resistance to degradation. For example, the RNA sequence may be modified with respect to its codon usage, for example, to increase translation efficacy and half-life of the RNA.

In some aspects, the RNA molecules may include one or more structural and/or chemical modifications or alterations which impart useful properties to the polynucleotide including, in some aspects, the lack of a substantial induction of the innate immune response of a cell into which the polynucleotide is introduced. As used herein, a “structural” feature or modification is one in which two or more linked nucleotides are inserted, deleted, duplicated, inverted or randomized in an RNA molecule without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some aspects, the RNA molecule may include one or more modified nucleotides in addition to any 5′ cap structure. Naturally occurring nucleoside modifications are known in the art.

In some aspects, the RNA molecule produced by the in vitro transcription reactions described herein includes from at least, at most, or about 20 to at least, at most, or about 100,000 nucleotides, or any range or value derivable therein (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000). In preferred aspects, the RNA molecule includes at least 100 nucleotides. For example, in some aspects, the RNA has a length between 100 and 15,000 nucleotides; between 7,000 and 16,000 nucleotides; between 8,000 and 15,000 nucleotides; between 9,000 and 12,500 nucleotides; between 11,000 and 15,000 nucleotides; between 13,000 and 16,000 nucleotides. In some aspects, the RNA has a length between about 1,600 nucleotides and 9,600 nucleotides. In preferred aspects, the RNA molecule that is the polynucleotide product of the in vitro transcription reaction described herein includes a gene of interest and a poly(A) tail. In some aspects, the RNA molecule further includes a 5′ UTR and a 3′ UTR.

In some aspects, the modified mRNA molecule encodes a single polypeptide antigen or, optionally, two or more of polypeptide antigens linked together in a way that each of the sequences retains its identity (e.g., linked in series) when expressed as an amino acid sequence. The polypeptide(s) generated from the modified mRNA may then be produced as a fusion polypeptide or engineered in such a manner to result in separate polypeptide or peptide sequences. In preferred aspects, the modified mRNA molecule encodes a single polypeptide of interest.

In some preferred aspects, the RNA molecule is a saRNA. “Self-amplifying RNA,” “self-amplifying RNA,” and “replicon” refer to RNA with the ability to replicate itself. Self-amplifying RNA molecules may be produced by using replication elements derived from, e.g., alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNAs. These daughter RNAs, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the antigen. The overall result of this sequence of transcriptions is an amplification in the number of the introduced saRNAs and so the encoded gene of interest, e.g., a viral antigen, becomes a major polypeptide product of the cells.

In some aspects, the self-amplifying RNA includes at least one or more genes selected from any one of viral replicases, viral proteases, viral helicases and other nonstructural viral proteins. In some aspects, the self-amplifying RNA may also include 5′- and 3′-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES).

In preferred aspects, the self-amplifying RNA molecule is not encapsulated in a virus-like particle. Self-amplifying RNA molecules described herein may be designed so that the self-amplifying RNA molecule cannot induce production of infectious viral particles. This may be achieved, for example, by omitting one or more viral genes encoding structural proteins that are necessary to produce viral particles in the self-amplifying RNA. For example, when the self-amplifying RNA molecule is based on an alpha virus, such as Sindbis virus (SINV), Semliki Forest virus and Venezuelan equine encephalitis virus (VEEV), one or more genes encoding viral structural proteins, such as capsid and/or envelope glycoproteins, may be omitted.

In some aspects, a self-amplifying RNA molecule described herein encodes (i) an RNA-dependent RNA polymerase that may transcribe RNA from the self-amplifying RNA molecule and (ii) a polypeptide of interest, e.g., a viral antigen. In some aspects, the polymerase may be an alphavirus replicase e.g., including alphavirus protein nsP4. In some aspects, the self-amplifying RNA molecules described herein may include one or more modified nucleotides (e.g., pseudouridine, N6-methyladenosine, 5-methylcytidine, 5-methyluridine).

The saRNA construct may encode at least one non-structural protein (nsP), disposed 5′ or 3′ of the sequence encoding at least one peptide or polypeptide of interest. Preferably, the sequence encoding at least one nsP is disposed 5′ of the sequences encoding the peptide or polypeptide of interest. Thus, preferably the sequence encoding at least one nsP is disposed at the 5′ end of the RNA construct. In some aspects, at least one non-structural protein encoded by the RNA construct may be the RNA polymerase nsP4. Preferably, the saRNA construct encodes nsP1, nsP2, nsP3 and nsP4. As is known in the art, nsP1 is the viral capping enzyme and membrane anchor of the replication complex (RC). nsP2 is an RNA helicase and the protease responsible for the ns polyprotein processing. nsP3 interacts with several host proteins and may modulate protein poly- and mono-ADP-ribosylation. nsP4 is the core viral RNA-dependent RNA polymerase. In some aspects, the polymerase may be an alphavirus replicase, e.g., comprising one or more of alphavirus proteins nsP1 nsP2, nsP3 and nsP4.

Whereas natural alphavirus genomes encode structural virion proteins in addition to the non-structural replicase polypeptide, in some aspects, the self-amplifying RNA molecules do not encode alphavirus structural proteins. In some aspects, the self-amplifying RNA may lead to the production of genomic RNA copies of itself in a cell, but not to the production of RNA that includes virions. Without being bound by theory or mechanism, the inability to produce these virions means that, unlike a wild-type alphavirus, the self-amplifying RNA molecule cannot perpetuate itself in infectious form. The alphavirus structural proteins which are necessary for perpetuation in wild-type viruses are absent from self-amplifying RNAs of the present disclosure and their place is taken by gene(s) encoding the protein of interest, such that the subgenomic transcript encodes the immunogen rather than the structural alphavirus virion proteins.

In some aspects, the self-amplifying RNA molecule may have two open reading frames. The first (5′) open reading frame encodes a replicase; the second (3′) open reading frame encodes a polypeptide comprising an antigen of interest. In some aspects the RNA may have additional (e.g., downstream) open reading frames, e.g., to encode further antigens or to encode accessory polypeptides.

Optionally, self-amplifying RNA molecules described herein may also be designed to induce production of infectious viral particles that are attenuated or virulent, or to produce viral particles that are capable of a single round of subsequent infection.

When delivered to a vertebrate cell, a self-amplifying RNA molecule may lead to the production of multiple daughter RNAs by transcription from itself (or from an antisense copy of itself). The self-amplifying RNA may be directly translated after delivery to a cell, and this translation provides an RNA-dependent RNA polymerase which then produces transcripts from the delivered RNA, thereby producing multiple daughter RNAs. These RNA molecules are antisense relative to the delivered RNA and may be translated themselves to provide in situ expression of a gene product, or may be transcribed to provide further transcripts with the same sense as the delivered RNA which are translated to provide in situ expression of the gene product.

In some aspects, the saRNA molecule is alphavirus-based. Alphaviruses include a set of genetically, structurally, and serologically related arthropod-borne viruses of the Togaviridae family. Exemplary viruses and virus subtypes within the alphavirus genus include Sindbis virus, Semliki Forest virus, Ross River virus, and Venezuelan equine encephalitis virus. As such, the self-amplifying RNA described herein may incorporate an RNA replicase derived from any one of Semliki Forest virus (SFV), Sindbis virus (SINV), Venezuelan equine encephalitis virus (VEEV), Ross-River virus (RRV), or other viruses belonging to the alphavirus family. In some aspects, the self-amplifying RNA described herein may incorporate sequences derived from a mutant or wild-type virus sequence, e.g., the attenuated TC83 mutant of VEEV has been used in saRNAs.

Alphavirus-based saRNAs are (+)-stranded saRNAs that may be translated after delivery to a cell, which leads to translation of a replicase (or replicase-transcriptase). The replicase is translated as a polyprotein which auto-cleaves to provide a replication complex which creates genomic (−)-strand copies of the (+)-strand delivered RNA. These (−)-strand transcripts may themselves be transcribed to give further copies of the (+)-stranded parent RNA and also to give a subgenomic transcript which encodes the desired gene product. Translation of the subgenomic transcript thus leads to in situ expression of the desired gene product by the infected cell. Suitable alphavirus saRNAs may use a replicase from a Sindbis virus, a Semliki Forest virus, an eastern equine encephalitis virus, a Venezuelan equine encephalitis virus, or mutant variants thereof.

In some aspects, the self-amplifying RNA molecule is derived from or based on a virus other than an alphavirus, preferably, a positive-stranded RNA virus, and more preferably a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

In some aspects, the self-amplifying RNA molecules described herein are larger than other types of RNA (e.g., mRNA). Typically, the self-amplifying RNA molecules described herein include at least about 4 kb. For example, the self-amplifying RNA may include at least, at most, or about 5 kb, 6 kb, 7 kb, 8 kb, 9 kb, 10 kb, 11 kb, or 12 kb or more, or any range or value derivable therein. In certain examples, the self-amplifying RNA is about 4 kb to about 12 kb, about 5 kb to about 12 kb, about 6 kb to about 12 kb, about 7 kb to about 12 kb, about 8 kb to about 12 kb, about 9 kb to about 12 kb, about 10 kb to about 12 kb, about 11 kb to about 12 kb, about 5 kb to about 11 kb, about 5 kb to about 10 kb, about 5 kb to about 9 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb.

In some aspects, the self-amplifying RNA molecule may encode a single polypeptide antigen or, optionally, two or more of polypeptide antigens linked together in a way that each of the sequences retains its identity (e.g., linked in series) when expressed as an amino acid sequence. The polypeptides generated from the self-amplifying RNA may then be produced as a fusion polypeptide or engineered in such a manner to result in separate polypeptide or peptide sequences.

In some aspects, the self-amplifying RNA described herein may encode one or more polypeptide antigens that include a range of epitopes. Preferably epitopes capable of eliciting either a helper T-cell response or a cytotoxic T-cell response or both. In some aspects, the RNA molecule has a 3′ poly(A) tail, that is, a stretch of consecutive adenosine residues, that may be attached to the 3′ end of the RNA. The poly(A) tail may increase the half-life of the RNA molecule. The RNA molecule may further include a poly(A) polymerase recognition sequence (e.g., AAUAAA) near its 3′ end. In some aspects, the 3′ poly(A) tail has a stretch of at least 10 consecutive adenosine residues and at most 300 consecutive adenosine residues. Preferably, the RNA molecule includes at least 20 consecutive adenosine residues and at most 40 consecutive adenosine residues. In some preferred aspects, the RNA molecule includes about 40 consecutive adenosine residues. In some aspects, the RNA molecule includes about 80 consecutive adenosine residues. Poly(A) tails may play key regulatory roles in enhancing translation efficiency and regulating the efficiency of mRNA quality control and degradation. Short sequences or hyper-polyadenylation may signal for RNA degradation. Exemplary designs include a poly(A) tails of about 40 As, about 80 As. In some aspects, the RNA molecule further includes an endonuclease recognition site sequence immediately downstream of the poly(A) tail sequence.

In some aspects, the RNA molecule produced by the in vitro transcription reaction described herein is purified, e.g., including filtration that may occur via, e.g., ultrafiltration, diafiltration, or, e.g., tangential flow ultrafiltration/diafiltration.

### Capping of RNA Molecule

In some aspects, the methods of producing RNA molecules described herein further include capping uncapped RNA molecules by contacting the uncapped RNA molecules with a capping reaction system, which includes any one of guanylyltransferase, s-adenosyl-L-methionine (SAM), guanosine triphosphate (GTP), and 2′-O-methyltransferase, and any combination thereof, to produce a capped RNA molecule. In some aspects, the 5′ end of the RNA is capped with a modified ribonucleotide with the structure m7G (5′)ppp(5′) N (cap 0 structure) or a derivative thereof, which may be incorporated during RNA synthesis (co-transcriptional capping) or may be performed enzymatically after RNA transcription (post-transcriptional capping). In some preferred aspects, the 5′ end of the RNA molecule is capped with a modified ribonucleotide via an enzymatic reaction after RNA transcription. In some aspects, capping is performed after purification, e.g., tangential flow filtration, of the RNA molecule.

An exemplary enzymatic reaction for capping may include use of Vaccinia Virus Capping Enzyme (VCE) that includes mRNA triphosphatase, guanylyltransferase and guanine-7-methytransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2′-O-Methyltransferase which results in the generation of a cap 1 structure (m7Gppp [m2′-O] N), which may further increase translation efficacy.

In some aspects, the RNA molecule may be enzymatically capped at the 5′ end using Vaccinia guanylyltransferase, guanosine triphosphate and S-adenosyl-L-methionine to yield cap 0 structure. An inverted 7-methylguanosine cap is added via a 5′ to 5′ triphosphate bridge. Alternatively, use of a 2′-O-methyltransferase with Vaccinia guanylyltransferase yields the cap 1 structure where in addition to the cap 0 structure, the 2′-OH group is methylated on the first transcribed nucleotide. S-adenosyl-L-methionine (SAM) is a cofactor utilized as a methyl transfer reagent. In some preferred aspects, RNase inhibitor is not included in the enzymatic capping reaction. In a preferred aspect, the enzymatic capping reaction step is performed under constant mixing. In another aspect, the RNA molecule is not co-transcriptionally capped.

Non-limiting examples of 5′ cap structures are those which, among other things, have enhanced binding of cap binding polypeptides, increased half-life, reduced susceptibility to 5′ endonucleases and/or reduced 5′ de-capping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme may create a canonical 5′-5′-triphosphate linkage between the 5′-terminal nucleotide of an mRNA and a guanine cap nucleotide wherein the cap guanine includes an N7 methylation and the 5′-terminal nucleotide of the mRNA includes a 2′-O-methyl. Such a structure is termed the Cap1 structure. This cap results in a higher translational-competency and cellular stability and a reduced activation of cellular pro-inflammatory cytokines, as compared, e.g., to other 5′ cap analog structures known in the art. Cap structures include, but are not limited to, 7 mG(5′)ppp(5′) N,pN2p (cap 0) and 7 mG (5′)ppp(5′) N1mpNp (cap 1). Cap 0 is a N7-methyl guanosine connected to the 5′ nucleotide through a 5′ to 5′ triphosphate linkage, typically referred to as m7G cap or m7Gppp. In the cell, the cap 0 structure is essential for efficient translation of the mRNA that carries the cap. An additional methylation on the 2′-O position of the initiating nucleotide generates Cap 1, or refers to as m7GpppNm-, wherein Nm denotes any nucleotide with a 2′-O methylation. In some aspects, the 5′ terminal cap includes a cap analog, for example, a 5′ terminal cap may include a guanine analog. Exemplary guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

In some aspects, the capping region may include a single cap or a series of nucleotides forming the cap. In this aspect the capping region may be from 1 to 10, e.g., at least, at most, or about 1, 2, 3, 4, 5, 6, 7, 8, 9, or 10 nucleotides, or any range or value therein, e.g., 2-9, 3-8, 4-7, 1-5, 5-10, or at least 2, or 10 or fewer nucleotides in length. In some aspects, the cap is absent.

In some aspects, the first and second operational regions may range from 3 to 40, e.g., at least, at most, or about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides, or any range or value derivable therein, e.g., 5-30, 10-20, 15, or at least 4, or 30 or fewer nucleotides in length and may comprise, in addition to a Start and/or Stop codon, one or more signal and/or restriction sequences.

In some aspects, the self-amplifying RNA molecules described herein have a 5′ cap (e.g., a 7-methylguanosine). This cap may enhance in vivo translation of the RNA. In some aspects, the self-amplifying RNA may include (in addition to any 5′ cap structure) one or more nucleotides having a modified nucleobase. In some aspects, the RNA molecule includes only phosphodiester linkages between nucleosides. In some aspects, the RNA molecule may include phosphoramidate, phosphorothioate, and/or methyl phosphonate linkages.

In some aspects, the modified mRNA molecules described herein have a 5′ cap (e.g., a 7-methylguanosine). This cap may enhance in vivo translation of the RNA. In some aspects, the modified mRNA may include (in addition to any 5′ cap structure) one or more nucleotides having a modified nucleobase. In some aspects, the RNA molecule includes only phosphodiester linkages between nucleosides. In some aspects, the RNA molecule may include phosphoramidate, phosphorothioate, and/or methyl phosphonate linkages.

In one aspect, the capping reaction system includes enzymatic 5′ capping that is performed as follows. The final 1× buffer conditions includes the following: at least, at most, or about 50 mM Tris HCl, pH 8, 5 mM KCl, 1 mM MgCl2, 0.5 mM GTP, 0.2 mM S-adenosyl-methionine and 1 mM dithiothreitol. In some aspects, the final 1× buffer does not include dithiothreitol.

In some aspects, the capping reaction occurs in the vessel in which the IVT reaction was performed. In such aspects, the IVT reaction is diluted between 3-fold and 10-fold, e.g., at least, at most, or about 3-fold, 4-fold, 5-fold, 6-fold, 7-fold, 8-fold, 9-fold, or 10-fold, before the capping reaction. In some aspects, the IVT reaction is diluted with Tris pH 7.0 buffer.

To degrade residual DNA template from the IVT reaction, DNase I can be added. In some aspects, DNase I is added at a concentration between at least, at most, or about 1 U/μg of DNA to 10 U/μg of DNA, e.g., 1, 2, 3, 4, 5, 6, 7, 8, 9, or 10 U/μg of DNA, or any range or value derivable therein. In addition to DNase I, CaCl₂) can be added as a co-factor for DNase I at a concentration between at least, at most, or about 0.1 mM to 4 mM, e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM, or any range or value derivable therein.

In some aspects, pyrophosphatase is added into the capping reaction. Pyrophosphatase assists with degrading pyrophosphate, which is the inhibitory by-product that is generated by the IVT reaction or by the capping reaction.

In some aspects, the capping reaction is conducted under 37° C. for 30 minutes to 2 hours, e.g., at least, at most, or about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 minutes, or any range or value derivable therein. In some aspects, the capping reaction is conducted at a temperature greater than 20° C. and less than 50° C., e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50° C.

In some aspects, the step of capping the uncapped RNA molecules results in at least, at most, or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% capped RNA molecules of the total of RNA molecules (capped and uncapped), or any range or value derivable therein. Purity may be determined as described herein, e.g., via reverse phase HPLC or Fragment analyzer or Bioanalyzer chip-based electrophoresis and measured by, e.g., peak area of full-length RNA molecule relative to total peak.

### Purification

In some aspects, the RNA molecule produced by the methods described herein may be contacted with DNase I and CaCl₂) to enzymatically digest DNA template following the in vitro transcription reaction. In some aspects, such as during a large scale (reaction volumes above 10 mL) operation, the IVT reaction may include DNase I and CaCl₂) additions as well as an additional treatment with EDTA and proteinase K. The EDTA may quench any cationic metal species, including magnesium, and the proteinase K may digest proteins present in the IVT reaction, reducing their size.

In some aspects, the methods described herein do not include contacting the RNA molecule produced by the methods described herein with DNase I to enzymatically digest DNA template following the in vitro transcription reaction.

In some aspects, the linear DNA template is removed from the in vitro transcription reaction system, for example, the DNA template is separated from the RNA molecule via chromatography. In some aspects, the RNA molecule binds to an affinity substrate while the DNA template flow through and is removed. In some aspects, the poly(A) capture-based affinity purification is oligo (dT) purification. For example, a polythymidine ligand may be immobilized to a derivatized chromatography resin. The mechanism of purification may involve hybridization of the poly(A) tail of the RNA molecule to the oligonucleotide ligand, wherein the DNA template will not bind. In preferred aspects, the RNA molecules that do not include Poly(A) stretches (abortive transcripts and other truncates formed during in vitro transcription) will not bind to the resin and will not form a duplex with the affinity ligand. Poly-adenylated RNA may then be eluted from the resin utilizing a low ionic strength buffer or a competitive binding oligonucleotide solution.

Preferably, purified material is substantially free of one or more impurities or contaminants including the linear DNA template and/or reverse complement transcription products described herein and for instance is at least 90%, 91%, 92%, 93%, 94%, 95%, 96%, or 97% pure; more preferably, at least 98% pure, and more preferably still at least 99% pure.

The method for production of an RNA molecule may include additional purification steps after the in vitro transcription, e.g., an ion exchange chromatography step, a hydrophobic interaction chromatography (HIC) step, a ceramic hydroxyapatite (CHA) chromatography step, and/or an ultrafiltration/diafiltration step. In a preferred aspect, said synthesizing of an RNA molecule of a given sequence is performed as a large-scale synthesis.

As used herein, the term “large scale” refers to a reaction yield of said RNA molecule in the order of milligram quantities, preferably of at least one gram. In some aspects, the in vitro transcription reaction is carried out in a bioreactor for large-scale synthesis of RNA. Therefore, the bioreactor may be adapted to carry out the methods described herein. In some aspects, the bioreactor is adapted to perform a batch process (in which all components for in vitro transcription are supplied at the start of the reaction), a fed-batch process (in which in vitro transcription components are provided initially, and additional in vitro transcription components are supplied, continuously or in increments, during the process with or without periodic product harvest), or a continuous (also referred to as a perfusion) process (in which a component (such as template and/or one or more enzymes) is immobilized and other components and/or products are added and removed continuously, step-wise, or intermittently throughout the process). In some aspects, a bioreactor for synthesizing RNA molecules of a given sequence, preferably on a large scale, is a modularly designed in vitro transcription reactor system, which includes a reaction module for carrying out in vitro RNA transcription reactions in a sequence-optimized reaction mix, a capture module for temporarily capturing the transcribed RNA molecules, and a control module for controlling the infeed of components of the reaction mix into the reaction module. The reaction module preferably includes a filtration membrane for separating nucleotides from the reaction mix, and the control of the infeed of the components of the reaction mix by the control module is based on a measured concentration of nucleotides.

As used herein, the term “bioreactor” refers to a chamber or test tube or column wherein an in vitro transcription reaction is carried out under specified conditions. The bioreactor may be thermally regulated to maintain accurately a specific temperature, usually between 25° C. and 45° C. In some aspects, the bioreactor maintains a temperature of about 25° C. In some aspects, the bioreactor maintains a temperature of about 30° C. In some aspects, the bioreactor maintains a temperature of about 37° C. The bioreactor may range in volume from at least 1, 10, 100, or 200 liters or more, or any volume in between. In some aspects, the volume of the IVT reaction is at least 10 L, 30 L or 50 L. In some aspects, the volume of the IVT reaction is at least 100 L. In some aspects, the DNA template is obtained from fermentation with cells grown during the initial growth phase (or growth phase) for a greater or lesser amount of time, depending on the needs of the practitioner and the requirement of the cells themselves. In some aspects, the cells are grown for a period of time sufficient to achieve a predefined cell density. In some aspects, the cells are grown for a period of time sufficient to achieve a predefined cell density of about 1×10⁶ cells/mL, about 5×10⁶ cells/mL, about 1×10⁷ cells/mL, about 5×10⁷ cells/mL, about 1×10⁸ cells/mL or about 5×10⁸ cells/mL. In some aspects, the cells are grown for a period of time sufficient to achieve a predefined optical density (OD₆₀₀) of about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300. In some aspects, the cells are grown for a period of time sufficient to achieve a cell density that is a given percentage of the maximal cell density that the cells would eventually reach if allowed to grow undisturbed. For example, the cells may be grown for a period of time sufficient to achieve a desired viable cell density of 1, 5, 10, 15, 20, 25, 30, 35, 40, 45, 50, 55, 60, 65, 70, 75, 80, 85, 90, 95 or 99 percent of maximal cell density. In some aspects, the cells are grown until the cell density does not increase by more than 15%, 14%, 13%, 12%, 11%, 10%, 9%, 8%, 7%, 6%, 5%, 4%, 3%, 2% or 1% per day of culture. In some aspects, the cells are grown until the cell density does not increase by more than 5% per day of culture. In some aspects the cells are allowed to grow for a defined period of time. For example, depending on the starting concentration of the cell culture and the intrinsic growth rate of the cells, the cells may be grown for 0, 1, 2, 3, 4, 5, 6, 7, 8, 9, 10, 11, 12, 13, 14, 15, 16, 17, 18, 19, 20 or more days, preferably for 4 to 10 days. The cell culture may be agitated or shaken during the process. In some aspects the bioreactor is configured to include agitation. The bioreactor may be a stirred-cell with provision for variable rates of stirring. In some aspects, the bioreactor comprises an agitation speed at least 0, 10, 20, 30, 40, 50, 60, 70, 80, 90, 100, 150, 200, 250, 300, 350, 400, 450, 500, 550, 600, 650, 700, 750, 800, 850, 900, 950, or 1,000 RPM (or the corresponding power/volume, tip speed, or mixing time). In some aspects, the bioreactor comprises an agitation speed of 150 RPM (equivalent to a power/volume of 1.3 W/m³). In some aspects, the bioreactor comprises an agitation speed of 250 RPM (equivalent to a power/volume of 5.9 W/m³). In some aspects, the bioreactor comprises an agitation speed of 600 RPM (equivalent to a power/volume of 71.7 W/m³). In some aspects, the bioreactor comprises a mixing time at least 1.0, 1.2, 1.3, 1.5, 2.0, 5.0, 10, 15, 20, 25, 30, 35, 40, 45, 50, 55, or 60 seconds. In some aspects, the bioreactor comprises an impeller tip speed of at least 0.1, 0.2, 0.3, 0.4, 0.5, 0.6, 0.7, 0.8, or 0.9 m/s. In some aspects, the bioreactor may be configured with an inflow or feed line and an exit port. In some preferred aspects, the bioreactor includes a filtration membrane for separating small and large molecules from the reaction mix, in particular for separating nucleotides, pyrophosphates and other low molecular weight components from the large (e.g., greater than at least 7,000 nt) mRNA product in the reaction mix. The introduction of a filtration membrane in such a flow system, for example an ultrafiltration membrane, may be used for separation of high molecular weight components, such as, e.g., polypeptides and/or polynucleotides, from low molecular weight components, such as nucleotides and pyrophosphates. In some aspects, the filtration membrane selectively retains an immobilized DNA template, the RNA polymerase and the synthesized RNA molecules in the reactor core of the reaction module whereas smaller molecules such as nucleotide triphosphates (NTPs) may pass the filtration membrane into a separate smaller compartment of the reaction module, i.e., the filtration compartment. The nucleotide concentration may then be measured, for example, by spectroscopic analysis in the separated fluid including the low molecular weight components. Alternatively, the nucleotide concentration may be measured by an online HPLC system. The application of an NTP mix in this reactor system allows the real-time measurement of the nucleotide concentration during the in vitro transcription reaction to monitor the progress of the in vitro transcription reaction. In some aspects, the bioreactor comprises static or agitated tube reactions. In some aspects, the bioreactor comprises a Sartorius AMBR system including but not limited to the AMBR15, AMBR250 modular, and AMBR250 HT systems. In some aspects, the bioreactor comprises an Eppendorf BioBlu system. In some aspects, the bioreactor comprises well plate reactions utilizing a Tecan liquid handler. In some aspects, the bioreactor comprises a vessel controlled by the Mettler Toledo EasyMax 102 or 402 synthesis workstations.

### Characterization and Analysis of the RNA Molecule

The RNA molecule produced by the methods described herein may be analyzed and characterized using various methods. Analysis may be performed before or after capping. Alternatively, analysis may be performed before or after poly(A) capture-based affinity purification. In another aspect, analysis may be performed before or after additional purification steps, e.g., anion exchange chromatography and the like. For example, RNA transcript integrity may be determined using electrophoresis (using the fragment analyzer capillary or Bioanalyzer chip systems) or through a reverse phase HPLC method. The fragment analyzer can in some instances automate capillary electrophoresis and HPLC. In other aspects, RNA template purity is analyzed using analytical reverse phase HPLC respectively. Capping efficiency may be analyzed using, e.g., total nuclease digestion followed by LC-UV or LC-MS quantitation of the dinucleotide cap species vs. uncapped GTP species. In vitro efficacy may be analyzed by, e.g., transfecting RNA molecule into a human cell line. Protein expression of the polypeptide of interest may be quantified using methods such as ELISA or flow cytometry. Immunogenicity may be analyzed by, e.g., transfecting RNA molecules into cell lines that indicate innate immune stimulation, e.g., PBMCs. Cytokine induction may be analyzed using, e.g., methods such as ELISA to quantify a cytokine, e.g., Interferon-α.

The method of producing RNA molecules described herein may produce an RNA molecule that is at least 30% full-length transcript, or at least, at most, or about 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% full-length transcript, or any range or value derivable therein. Purity may be determined as described herein, e.g., via reverse phase HPLC or Bioanalyzer chip-based electrophoresis and measure by, e.g., peak area of full-length RNA molecule relative to total peak.

### Genes of Interest

The DNA template and resulting RNA molecule of the present invention include a gene of interest. The gene of interest encodes a polypeptide of interest selected from, e.g., biologics, antibodies, vaccines, therapeutic polypeptides or peptides, cell penetrating peptides, secreted polypeptides, plasma membrane polypeptides, cytoplasmic or cytoskeletal polypeptides, intracellular membrane bound polypeptides, nuclear polypeptides, polypeptides associated with human disease, targeting moieties or those polypeptides encoded by the human genome for which no therapeutic indication has been identified but which nonetheless have utility in areas of research and discovery. The sequence for a particular gene of interest is readily identified by one of skill in the art using public and private databases, e.g., GenBank.

In some aspects, the RNA molecule includes a coding region for an antigen preferably derived from a pathogen associated with infectious disease which are preferably selected from antigens derived from the pathogens Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DENV-1, DENV-2, DENV-3 and DENV-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157: H7, 0111 and 0104: H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBOV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (including Influenza such as avian influenza and human influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, Coronavirus (e.g., SARS-CoV-2), Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In some aspects, the RNA molecules of the present disclosure encode a viral polypeptide or fragment thereof, including naturally occurring or engineered variants thereof, for prophylaxis against a virus in humans. In some aspects, the viral polypeptide does not comprise a coronavirus polypeptide. In some aspects, the viral polypeptide does not comprise a severe acute respiratory syndrome (SARS) virus polypeptide. In some aspects, the viral polypeptide does not comprise a SARS-CoV-2 polypeptide.

Thus, in some aspects, the RNA molecules of the present disclosure do not encode a coronavirus polypeptide or fragment thereof, including naturally occurring or engineered variants thereof. In some aspects, the RNA molecules of the present disclosure do not encode a SARS virus polypeptide or fragment thereof, including naturally occurring or engineered variants thereof. In some aspects, the RNA molecules of the present disclosure do not encode a SARS-CoV-2 virus polypeptide or fragment thereof, including naturally occurring or engineered variants thereof.

In further aspects, the RNA molecules of the present disclosure are not used for prophylaxis against a coronavirus in humans. In some aspects, the RNA molecules of the present disclosure are not used for prophylaxis against a SARS virus in humans. In some aspects, the RNA molecules of the present disclosure are not used for prophylaxis against SARS-CoV-2 in humans.

### EXAMPLES

Below are examples of specific aspects for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art.

### Example 1

### Creation of a dsDNA Mini-Circle by Assembly of Synthetic Fragments

A 3′ UTR and poly(A) sequence were amplified by PCR from pVV-0513, a plasmid containing a mod flu-HA transcription cassette. A kanamycin-resistant vector plasmid, PFE-pUC-Kan 3, was linearized by digestion. Two fragments (3′ UTR-pA and T7p-5′ UTR) were assembled into the linearized pUC-Kan backbone using the NEBUILDER® HIFI DNA Assembly Cloning Kit (NEW ENGLAND BIOLABS®) to produce plasmid pREG5. A fragment containing the regulatory sequences (3′ UTR, poly(A), T7 promoter, 5′ UTR) was cut out of pREG5.

The regulatory sequences are assembled with a synthetic antigen sequence obtained from IDT® to produce a dsDNA minimal circle template.

After assembly, the reaction is treated with T5 exonuclease to remove any remaining linear fragments, leaving only circular, dsDNA for use as a template for RCA.

The T5 exonuclease is removed by digestion with heat-labile proteinase K followed by heat inactivation of proteinase K.

### Example 2

### RCA Reaction with Circular dsDNA as the Starting Template

In some aspects, a further simplified process for mRNA production utilizes circular double-stranded DNA constructed synthetically that is amplified isothermally by multiple-strand displacing rolling circle amplification (RCA) via Phi29 DNA polymerase or mutant thereof.

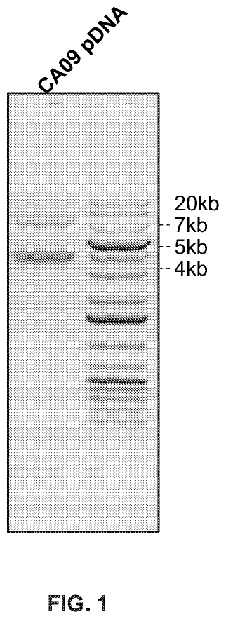

One to five ng of circular, supercoiled dsDNA template (containing a transcribable cassette) shown in FIG. 1 can be used in a rolling circle amplification (RCA) reaction (Table 1) to generate 10⁵-fold amplification of the template, yielding a dsDNA concatemer.

TABLE 1
RCA Reaction with EQUIPHI29 ™ DNA Polymerase

| | Final | |
|---|---|---|
| Reagents | concentration | |
| 10X EQUIPHI29 ™ reaction buffer | 1 | X |
| DTT | 4.00 | mM |
| Inorganic yeast Pyrophosphatase | 0.001-0.01 | U/μL |
| dATP | 1.00-2.00 | mM |
| dCTP | 1.00-2.00 | mM |
| dTTP | 1.00-2.00 | mM |
| dGTP | 1.00-2.00 | mM |
| 3′ exonuclease resistant random hexamer | 1-50 | μM |
| EQUIPHI29 ™ DNA polymerase | 0.50 | U/μL |
| circular dsDNA template | 1-5 | ng/mL |

Nuclease free water to reaction volume

The above mixture was mixed gently with or without heat-denaturation and then incubated for 3-6 hrs to overnight at 30-45° C. Post incubation, EQUIPHI29™ polymerase can be heat inactivated at 65° C. for 10 min. Preferably, the EQUIPHI29™ polymerase is not heat inactivated. The reaction can achieve 1.5-2 mg/ml of DNA.

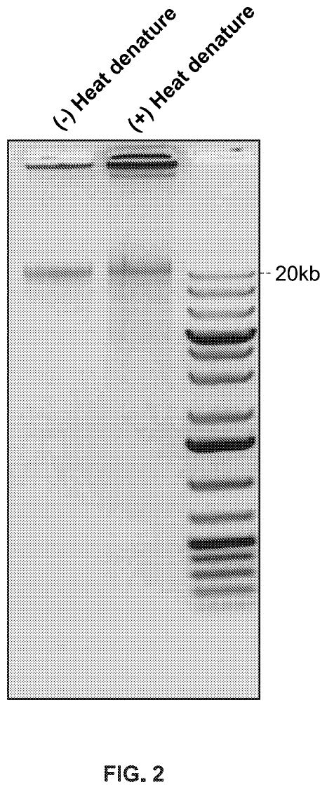

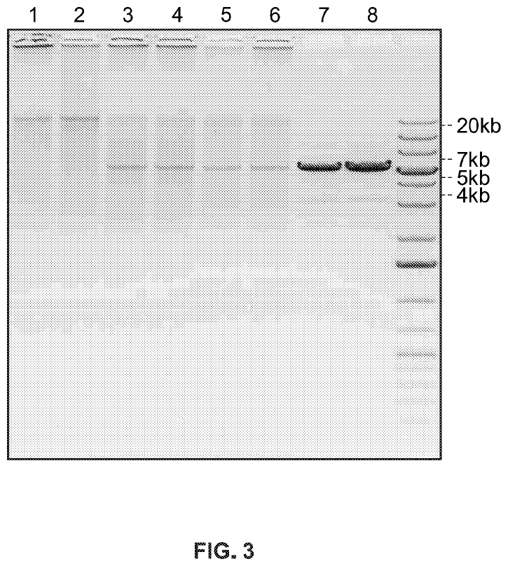

RCA DNA concatemers generated from the above reactions are shown in the agarose gel image in FIG. 2, without and with heat denaturation, and FIG. 3, Lanes 1-2 (without and with purification post RCA).

These experiments show that RCA reactions can be conducted at a temperature of 30-45° C. for either 3-6 hrs or overnight. Additionally, BSA can be removed and different dNTP concentrations and Phi29 polymerases used to successfully generate RCA templates for use in IVT reactions. Finally, a heat-denaturation step prior to incubation for RCA and a heat inactivation post-RCA, pre-linearization can be omitted from the RCA reaction process, with no impact on integrity of RNA produced from RCA-generated templates. Removing these two steps allow for commercial manufacturing ease and allow for an isothermal process, while still surprising producing quality mRNA as compared to plasmid DNA, as shown in the below Examples.

### Example 3

### Digestion of RCA DNA without Purification

Additionally, in some aspects, the RCA DNA of Example 2 generated from circular dsDNA template can be enzymatically digested by one or more restriction endonucleases to generate linearized DNA fragments for use as template for in vitro transcription reactions in a “one pot” reaction without purification of the RCA DNA prior to enzymatic digestion. Typical reactions require purification of DNA template, reconstitution of the DNA template in buffer, then enzyme digestion of the DNA template. As described herein, the entire reaction (i.e., amplification and linearization) can be performed in the same reaction vessel without losing material, since there is no need to transfer to different vessels for the different reaction steps.

RCA DNA can be digested with a restriction endonuclease to produce linearized dsDNA without the need for DNA purification or additional buffering reagents. 2-3 U of SapI (10 U/μL, NEB™, #R0659) per microgram of RCA DNA was pipetted into the RCA reaction vessel and incubated 8 hrs to overnight at 37° C. in a heat block. As shown in FIG. 3, lanes 3 and 4 (no purification prior to linearization) compared to lanes 5 and 6 (with purification), the cut products can be viewed using agarose gel electrophoresis.

### Example 4

### RCA Reaction with RCA DNA as the Starting Template

Optionally, in some aspects, a sequential multi-step RCA reaction—with RCA DNA from prior RCA reactions used as starting template for further rounds of rolling circle amplification—is used to achieve up to 10¹⁰ increased fold DNA amplification from as little as 1-5 ng of template DNA starting material.

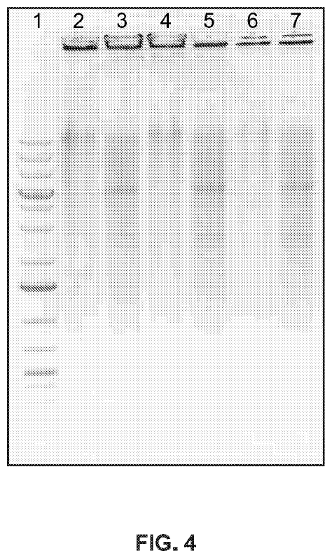

RCA DNA concatemers generated from Example 3 were used as a template for a second RCA reaction using RCA DNA as the starting template. The second RCA reaction was identical to the reactions described in Table 1 except that 5 ng of the RCA DNA obtained from the reactions in Example 3 (RCA DNA template 1) is substituted for circular dsDNA plasmid (plasmid template) as the template for RCA to produce RCA DNA reaction template 2. RCA DNA concatemers generated from the second RCA reaction are shown in the agarose gel image in FIG. 4, Lanes 4 (uncut template) and 5 (restriction digested, linearized template).

RCA DNA reaction template 2 generated from the second RCA reaction was then used as a template for a third RCA reaction. The third RCA reaction was identical to the second RCA reaction except that 5 ng of RCA DNA reaction template 2 is substituted for RCA DNA reaction template 1 as the template for RCA to produce RCA reaction template 3. RCA DNA concatemers generated from the second RCA reaction are shown in the agarose gel image in FIG. 4, Lanes 6 (uncut template) and 7 (restriction digested, linearized template).

These results demonstrate that an RCA reaction using RCA DNA as the starting template can be repeated up to 3 times without loss of yield or quality.

### Example 5

### IVT Using Linearized DNA as Template

Linearized DNA, generated from either circular dsDNA (plasmid DNA or minicircle) or RCA DNA template, can be used as a template for in vitro transcription.

The linearized RCA DNA from Examples 2-4 was used as an IVT template to generate mRNA. Linearized RCA DNA was purified using ethanol/2.5 M NH₄CH₃CO₂ precipitation and re-suspended in nuclease free water. The RCA DNA was then used as a template in an IVT reaction as shown in Table 2 below (Trilink protocol). RCA DNA was supplemented at 0.05 mg/mL final concentration.

TABLE 2
IVT Reaction with Linear RCA DNA as Template

| Reagents | Final concentration | |
|---|---|---|
| Tris buffer, pH 8.0 | 40 | mM |
| NTP mix | 20 | mM |
| Inorganic yeast Pyrophosphatase | 0.002 | U/μL |
| Rnase inhibitor | 0.5 | U/μL |
| T7 polymerase | 8 | U/μL |
| MgOAc | 16.5 | mM |
| Spermidine | 2 | mM |
| DTT | 10 | mM |
| RCA DNA | 50 | ng/μL |

Nuclease free water to reaction volume

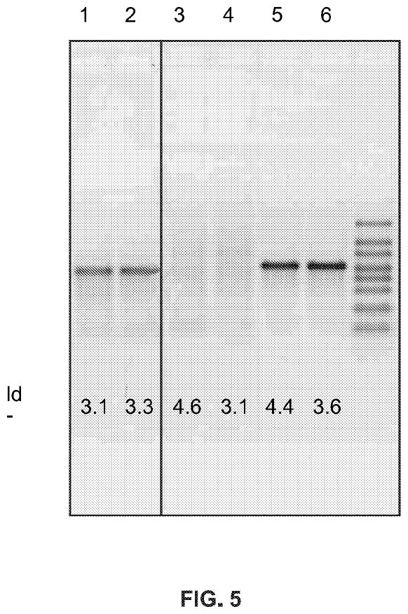

The above reaction was gently vortexed and incubated at 37° C. for 120 min. DNaseI and CaCl₂) were added, and the reaction mixture was incubated at 37° C. The reaction was purified by LiCl precipitation. The reaction mixture was centrifuged, the resulting pellet was washed with ethanol, and the reaction was further centrifuged. The final pellet was re-suspended in water and RNA concentration measured on nanodrop. As shown in FIG. 5, the products can be viewed using agarose gel electrophoresis without further purification.

### Example 6

### Purification of mRNA Generated from Linearized RCA DNA Template to Increase RNA Integrity

In some aspects, the in vitro transcription reaction mixture produced according to Example 5 can be purified by oligo (dT) affinity capture in order to further enrich for full length mRNA product. Purification of mRNA generated from linearized RCA DNA template using oligo (dT) capture can increase RNA integrity equal to or beyond RNA integrity generated from linearized plasmid DNA.

Polyadenylated mRNA generated from linearized RCA DNA can be enriched for full length transcripts by capture using oligo (dT) magnetic beads (DYNABEADS™ Oligo (dT) 25, INVITROGEN™). RNA was re-suspended in an equal volume of binding buffer (Tris-HCl, pH 7.5; LiCl; EDTA) and mixed with an equal volume of magnetic beads in binding buffer. The mixture was incubated at room temperature with rotation, and the unbound fraction was removed. The beads were washed twice with wash buffer B (Tris-HCl, pH 7.5; LiCl; EDTA) before RNA was eluted using Tris-HCl, pH 7.5. In some aspects, RNA is then precipitated in LiCl, followed by centrifugation at 4° C. The resulting pellet is then washed with ethanol and further centrifuged at 4° C., and the final pellet is re-suspended in water.

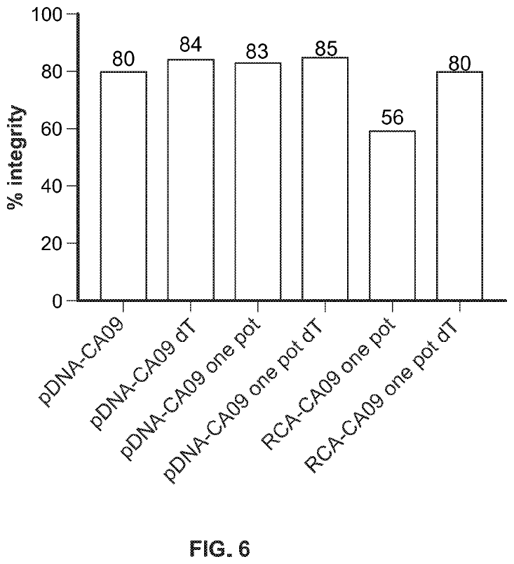

The integrity of the mRNA was assessed by fragment analysis. Oligo (dT)-selected mRNA that was transcribed from plasmid DNA was used as a control. As shown in FIG. 6, after oligo (dT) selection, RNA integrity of mRNA from RCA DNA templates was improved to equal that obtained from plasmid DNA-templated RNA. Thus, an oligo (dT) purification step after IVT reactions is a creative solution for enrichment of RNA yield having high integrity.

### Example 7

### Sequential, Single-Vessel Reaction to Generate Uncapped mRNA

An RCA reaction with circular dsDNA (plasmid or mini-circle) followed by restriction enzyme linearization and IVT can be performed sequentially, in a single reaction vessel, generating mRNA without any previous purification steps. The process allows for DNA-directed synthesis of RNA molecules of any sequence, ranging in size from short oligonucleotides to several kilobases.

In some aspects, DNA, either plasmid or DNA generated from an RCA reaction, can be linearized and used as template for IVT reaction in a step-wise reaction in a single vessel without the need for purification or volume transfer. For the RCA single vessel reaction, template can be as little as 1 ng of circular supercoiled dsDNA. Heat denaturation of template DNA is not required. The RCA reaction components for such a step-wise single-vessel “one pot” RCA reaction are as in Table 1 above.

The reaction mixture is gently vortexed and incubated for 25 hrs at 30° C. Then, the RCA reaction is digested in the same vessel with a restriction endonuclease to produce linearized dsDNA without the need for DNA purification or additional buffering reagents. 2-3 U of SapI (10 U/L, NEB, #R0659) per microgram of RCA DNA is pipetted into the RCA reaction vessel and incubated 8 hrs to overnight at 37° C. As shown in FIG. 3, lane 3, the cut products from the duplicate reaction can be viewed by agarose gel electrophoresis without any purification.

The entirety of the digestion reaction (either linearized plasmid DNA or linearized RCA DNA) can be used in a subsequent IVT step in the same vessel without need for purification. IVT reagents were added directly to the enzyme-digested reaction according to Table 2, above. Nuclease free water was used to adjust DNA concentration to 50 ng/uL for IVT. Reagents were gently mixed, and the reaction was incubated at 37° C. for 120 min. DNaseI and CaCl₂) were added to final concentration as 5 U DNaseI/μg RCA DNA and 10 mM of CaCl₂), and the reaction was incubated for additional 40 min at 37° C. 500 mM EDTA and 0.6 U/uL of ProteinaseK were added to a concentration of 50 mM EDTA and 0.001 U/μL of ProteinaseK, and the reaction was incubated for an additional 10 min at 37° C.

The reaction can be purified by LiCl precipitation. The mixture is centrifuged at 4° C., the resulting pellet is washed with ethanol and further centrifuged at 4° C., and the final pellet is re-suspended in water and heated at 50° C. for 10 minutes to dissolve RNA into solution. Concentration is measured on nanodrop and typically generates 2.0 to 5.0 mg/ml of uncapped RNA.

The integrity of the resulting RNA was assessed using fragment analysis. RNA produced using linearized plasmid DNA was used as a control. Upon capture and purification with oligo (dT), RNA integrity is further increased beyond precipitation alone.

### Example 8

### In Vitro Transcription Reactions

Transcription buffer components were varied to determine the most impactful levers to the in vitro transcription (IVT) performance and product quality attributes. The reaction components and the concentration ranges studied are shown in Table 3 below.

Reactions were carried out in a high-throughput small-scale model. A list of the constant parameters throughout the experiment can be found in Table 37. RNA samples were purified by precipitation and concentration was measured by UV. RNA integrity was determined by Fragment Analyzer (FA). Parameter ranges were identified based on production of RNA≥1.85 mg/mL, accommodation to the model, and economic considerations. Table 3 below shows reagents and their studied ranges. A 2-level factorial Design of Experiment (DoE) was generated with 16 of the possible 64 high-low combinations of each factor. 2 replicate center-points were also run.

TABLE 3
DOE1 Factors and Ranges

| Process | | | | Studied | Low | Center | High | One |
|---|---|---|---|---|---|---|---|---|
| Parameter | Name | Units | Target | Range | (−1) | (0) | (+1) | Unit* |
| A | Tris | mM | 40 | 20-60 | 20 | 40 | 60 | 20 |
| B | Spermidine | mM | 2.5 | 0-5 | 0 | 2.5 | 5 | 2.5 |
| C | DTT | mM | 10 | 0-20 | 0 | 10 | 20 | 10 |
| D | Triton | % | 0.005 | 0-0.01 | 0 | 0.005 | 0.01 | 0.005 |
| E | DMSO | % | 0.835 | 0-1.67 | 0 | 0.835 | 1.67 | 0.835 |
| F | PEG-8000 | % | 2 | 0-4 | 0 | 2 | 4 | 2 |

*Note:
One unit represents the actual number of units for one coded unit.

Models capable of detecting all main effects and two factor interactions were generated for UV concentration and integrity. The criteria of p-value<0.05 was used to determine whether parameters were significant.

The ranges in RNA concentration and integrity observed throughout the experiment are listed in Table 4. The integrity and concentration values were acceptable across the tested ranges, which indicates that the process can operate safely within the ranges.

TABLE 4
DOE1 Responses

| | | | | | Standard |
|---|---|---|---|---|---|
| Response | Units | Minimum | Maximum | Mean | Deviation |
| UV Concentration | mg/mL | 3.96 | 4.41 | 4.14 | 1.11 |
| Integrity | % | 80.97 | 92.33 | 87.58 | 3.75 |

The fit statistics of the concentration and integrity models are displayed in Table 5. Based on analysis of the R² values, a relatively strong model for integrity was developed.

TABLE 5
DOE1 Model Overview

| Model | R² | Adjusted R² | Predicted R² |
|---|---|---|---|
| UV Concentration | 0.6666 | 0.5777 | 0.3396 |
| Integrity | 0.9136 | 0.9034 | 0.8838 |

In the model for UV concentration shown in Table 6, only PEG-8000 concentration and an interaction term between Tris and spermidine were statistically significant. The coefficient of each of these parameters was very small, so varying each parameter within the tested ranges resulted in negligible changes in concentration. Thus, it was determined that these terms were statistically significant, but not practically significant in their tested ranges.

TABLE 6
DOE1 UV Concentration Model

| | | Statistically | Coefficient | Practically |
|---|---|---|---|---|
| Parameter | p-Value | Significant? | Estimate | Significant? |
| A - Tris | 0.6664 | N | −0.0080 | N |
| B - Spermidine | 0.0604 | N | −0.0371 | N |
| F - PEG-8000 | 0.0172 | Y | −0.0465 | N |
| AB (Tris and | 0.0006 | Y | 0.0792 | N |
| Spermidine | | | | |
| interaction term) | | | | |
| Intercept | N/A | N/A | 4.14 | N/A |

In the model for integrity, Tris and Spermidine were statistically significant. However, Tris concentration (20-60 mM) was not practically significant as the coefficient estimate for the data was within FA assay variability. Surprisingly, addition of spermidine correlated with a significant drop of integrity. Spermidine is usually considered a critical component for in vitro transcription reactions [Kartje et al., J Biol Chem 296 (2021)]. Spermidine is a polyamine that is believed to assist the polymerase in disassociating from a DNA molecule and initiating RNA synthesis on a new DNA molecule. As shown in Table 6 and Table 7, spermidine can be removed from the reaction for the purpose of boosting integrity without significantly impacting concentration.

TABLE 7
DOE1 Integrity Model

| | | Statistically | Coefficient | Practically |
|---|---|---|---|---|
| Parameter | p-Value | Significant? | Estimate | Significant? |
| A - Tris | 0.0053 | Y | −0.9300 | N |
| B - Spermidine | <0.0001 | Y | −3.78 | Y |
| Intercept | N/A | N/A | 4.14 | N/A |

Interestingly, DTT concentration was not statistically significant in the tested range. DTT is a reducing agent that is commonly included in IVT reactions to protect enzymes such as T7 polymerase from protein oxidation [Kartje et al., J Biol Chem 296 (2021)]. The T7 polymerase used in these reactions contained small amounts of DTT to protect the enzyme for long-term storage, but it appeared that additional DTT was not needed to protect the T7 during reaction progression.

As demonstrated herein, the process can be run without spermidine for the purpose of boosting integrity. Further, a streamlined IVT matrix without spermidine, DTT, Triton, DMSO, and PEG-8000 is feasible for the process, leading to simplified manufacturing operations, improved stability of intermediate solutions, and reduced costs.

### Example 9

### Enhanced In Vitro Transcription Reactions

The following factors were evaluated to determine the most significant in IVT and which warrant further optimization studies: concentration of each NTP, T7 concentration, pyrophosphatase concentration, initial pH, Mg/NTP ratio, time, and temperature. Mg/NTP ratio was selected as a factor as opposed to Mg concentration because there is a codependence between Mg and NTP levels. NTPs are known to chelate Mg ions and if a certain Mg/NTP ratio threshold is exceeded, mRNA production would be significantly reduced [Kartje et al., J Biol Chem 296:100175 (2021); Young et al., Biotechnol Bioeng 56 (2): 210-220 (1997); Thomen et al., Biophysical Journal 95 (5) 2423-2433 (2008)].

As in Example 8, ranges were determined based on results of IVT reactions and then further adjusted to ensure response in the model and minimize cost.

Reactions were carried out in a small-scale high throughput model with varying levels of ATP, CTP, GTP, and pUTP; T7 concentration; pyrophosphatase concentration; initial reaction pH; and Mg acetate. Mg acetate was varied dependent on NTP concentrations in ratios ranging from 0.8-2.2. This equated to an operating range of 12.8-110 mM Mg. For simplicity, the ratio of ATP:CTP:GTP:pUTP was maintained at 1:1:1:1; e.g. 16 mM Total NTPs equated to 4 mM ATP, 4 mM CTP, 4 mM GTP, and 4 mM pUTP. Reactions were incubated at varying temperatures for varying periods of time. All other consistent parameters can be found in Table 41. RNA samples were purified by precipitation and concentration was measured by UV.

The levels of each tested parameter are captured in Table 8. The design consisted of 16 of the 128 possible combinations of high/low of each parameter and contained 2 center replicates.

TABLE 8
DOE2 Factors and Ranges

| Process | | | | Studied | Low | Center | High | One |
|---|---|---|---|---|---|---|---|---|
| Parameter | Name | Units | Target | Range | (−1) | (0) | (+1) | Unit* |
| A | Total NTP | mM | 33 | 16-50 | 16 | 33 | 50 | 17 |
| B | T7 | U/ml | 8000 | 4000-12000 | 4000 | 8000 | 12000 | 4000 |
| C | Pyrophosphatase | U/ml | 4.125 | 0.25-8 | 0.25 | 4.125 | 8 | 3.875 |
| D | pH | pH | 8.0 | 7.5-8.5 | 7.5 | 8.0 | 8.5 | 0.5 |
| E | Mg Ratio | mM | 1.5 | 0.8-2.2 | 0.8 | 1.5 | 2.2 | 0.7 |
| | | Mg/mM | | | | | | |
| | | NTP | | | | | | |
| F | Time | Min | 150 | 60-240 | 60 | 150 | 240 | 90 |
| G | Temperature | (deg C.) | 37 | 30-44 | 30 | 37 | 44 | 7 |

*Note:
One unit represents the actual number of units for one coded unit.

Models were generated in Design Expert for UV concentration that captured all main effects and two-factor interactions. The criteria of p-value<0.05 was used to determine whether each parameter was significant.

The performance of the model generated in Design Expert for UV concentration is displayed in Table 9. The predicted R² was low and was not in line with the adjusted R²; this indicates that the model is limited in its ability to predict concentration.

TABLE 9
DOE2 Model Overview

| Model Name | R² | Adjusted R² | Predicted R² |
|---|---|---|---|
| UV Concentration | 0.7204 | 0.6330 | 0.4300 |

One possible model for the data is shown in Table 10. The midpoint controls, which consisted of the “Target” ranges recorded in Table 8 had higher concentrations than the rest of the conditions. This resulted in significant curvature in the data, and this is indicated by the high coefficient present in the A² term. Many different models were possible, including quadratic models for any of the tested parameters (likely because all factors were centered in the midpoint controls). While most of the parameters could have been incorporated in the quadratic model, there were already plans to test pyrophosphatase, initial pH, and time through other experiments. The remainder of the parameters were carried forward for further optimization.

TABLE 10
DOE2 UV Concentration Model

| | | Statistically | Coefficient | Practically |
|---|---|---|---|---|
| Parameter | p-Value | Significant? | Estimate | Significant? |
| A - Total NTP | 0.0627 | N | −0.6879 | Y |
| B -T7 | 0.0282 | Y | 0.8701 | Y |
| F - Time | 0.0255 | Y | 0.8806 | Y |
| G - Temperature | 0.0330 | Y | 0.8418 | Y |
| A² | 0.0005 | Y | −4.39 | Y |
| Intercept | N/A | N/A | 6.67 | N/A |

RNA was successfully produced across all tested ranges of total NTP, T7, pyrophosphatase, initial pH, Mg ratio, time, and temperature.

### Example 10

### Refinement of Enhanced In Vitro Transcription Reactions

Based on the results of the experiments described in the previous examples, it was determined that NTP levels, T7 RNA polymerase concentration, Mg concentration, and temperature would be further optimized. The goal of this experiment was to optimize the setpoints of each of these parameters for RNA concentration. Mg was tested as a separate parameter, as opposed to a combined Mg:NTP ratio, to read the separate effects of Mg and NTP concentrations.

Reactions were carried out in a high throughput small-scale model with varying levels of ATP, CTP, GTP, and pUTP; T7 concentration; and Mg acetate concentration. For simplicity, the ratio of ATP:CTP:GTP:pUTP was fixed at 1:1:1:1. For instance, a total NTP concentration of 20 mM refers to 5 mM each of ATP, CTP, GTP, and pUTP. Further description of the fixed parameters can be found in Table 41. All varied parameters are displayed in Table 11. A modified full factorial DOE design was employed to test the parameters at the specified ranges. The design consisted of 6 centers, 8 axials, and 16 factorial points. For experimental simplicity, runs 4 and 15 were run at 37° C. and 30° C., respectively, as opposed to at a setpoint outside of the design space. RNA samples were purified by precipitation and concentration was approximated by UV. Percent integrity was determined by Fragment Analyzer (FA).

TABLE 11
DOE3 Design

| Process | | | | | | Low | High | One |
|---|---|---|---|---|---|---|---|---|
| Parameter | Name | Units | Min | Max | Mid | Alpha | Alpha | Unit* |
| A | Total NTP | mM | 20 | 36 | 28 | 2 | 2 | 8 |
| B | T7 | U/mL | 8000 | 14000 | 11000 | 2 | 2 | 3000 |
| C | Mg | mM | 20 | 36 | 28 | 2 | 2 | 8 |
| D | Temperature | (deg C.) | 30 | 37 | 33.5 | 1 | 1 | 3.5 |

*Note:
One unit represents the actual number of units for one coded unit.

Models to optimize RNA concentration were generated in Jmp and Design Expert based on the data collected in this experiment. The R², Adjusted R², and Predicted R² were high, as shown in Table 12, which suggests that the model fit well.

TABLE 12
DOE3 Model Performance

| Model Name | R² | Adjusted R² | Predicted R² |
|---|---|---|---|
| UV Concentration | 0.9090 | 0.8680 | 0.7506 |

The model for UV concentration from Design Expert can be found in Table 13. All four of the tested parameters were statistically significant, as p<0.05 for each parameter. Prediction profilers were generated in Jmp. The ranges displayed within each plot of the prediction profile were significant and could not be accounted for by experimental variability. Interaction terms were identified for the terms total NTP with Mg and Mg with temperature.

TABLE 13
DOE3 Model for UV Concentration

| | | Statistically | Coefficient |
|---|---|---|---|
| Parameter | p-Value | Significant? | Estimate |
| A - Total NTP | 0.0002 | Y | −0.7294 |
| B -T7 | 0.0047 | Y | 0.5053 |
| C - Mg | <0.0001 | Y | 0.9381 |
| D - Temp | 0.0002 | Y | 0.8317 |
| AC | 0.0001 | Y | 0.9286 |
| CD | 0.0089 | Y | 0.5642 |
| A² | <0.0001 | Y | −0.8826 |
| C² | <0.0001 | Y | −0.7149 |
| D² | 0.0047 | Y | −0.9357 |
| Intercept | N/A | N/A | 5.33 |

Prediction profilers were generated in Jmp to predict at what levels of each parameter mRNA concentration would be maximized. The desirability profiles suggest that total ATP, CTP, GTP, and pUTP is optimized at approximately 28 mM, T7 is optimized at higher concentrations, Mg is maximized at around 33-36 mM, and temperature is optimized at approximately 36 degrees C.

The best-performing conditions from this experiment was termed “DOE3 F1” and contained 36 mM total ATP/CTP/GTP/pUTP; 14,000 U/mL T7; and 36 mM Mg. The reaction temperature was 37 degrees C. This condition had a resulting mRNA concentration of 6.9 mg/mL and integrity at 89%. However, the multiple successful runs with varied parameters demonstrates that the process is robust and can be run at wide ranges.

### Example 11

### T7 and Temperature

Based on the previous examples, it was identified that reaction yield is optimized at higher levels of T7 RNA polymerase. For this reason, the impact of higher T7 levels was investigated in this experiment. Additionally, it was of interest to investigate temperature further as a parameter in the IVT reaction. Further, it is feasible that lowering temperature may boost integrity.

The experiment was performed in a high throughput small-scale model. The condition named DOE3 Opt described in Table 37 was run as a control in both one factor at a time (OFAT) experiments and is labeled as Condition A in Table 14 and Table 15. In the remaining conditions, T7 or temperature was manipulated.

RNA samples were purified by precipitation and concentration was measured by UV. Percent integrity was determined by Fragment Analyzer (FA). As T7 concentration increased, the resulting mRNA concentration remained comparable within experimental variability. A 6% drop in integrity was observed when T7 concentration was increased from 17000 to 40000 U/mL. These results are displayed in Table 14 and Table 15.

TABLE 14
T7 Concentration OFAT Results

| | | | Resulting | |
|---|---|---|---|---|
| | | T7 | mRNA | |
| | Temperature | Concentration | Concentration | Integrity |
| Condition | (degrees C.) | (U/mL) | (mg/mL) | (%) |
| A (DOE3 | 36 | 17000 | 5.2 | 87 |
| Opt) | | | | |
| B | 36 | 25000 | 5.7 | 84 |
| C | 36 | 40000 | 5.2 | 81 |

As temperature was decreased from 36° C. to 32° C., concentration and integrity were not significantly impacted; all results were within expected assay variability. These results are displayed in Table 15.

TABLE 15
Temperature OFAT Results

| | | | Resulting | |
|---|---|---|---|---|
| | T7 | | mRNA | |
| | Concentration | Temperature | Concentration | Integrity |
| Condition | (U/mL) | (degrees C.) | (mg/mL) | (%) |
| A (DOE3 | 17000 | 36 | 5.2 | 87 |
| Opt) | | | | |
| B | 17000 | 34 | 4.4 | 88 |
| C | 17000 | 32 | 5.0 | 89 |

This experiment demonstrates that the process is capable of producing quality mRNA within the temperature and T7 polymerase ranges tested.

### Example 12

### Linearized Plasmid DNA Concentration

This experiment determined whether starting DNA concentration impacted integrity and yield and assessed a range of 0.025-0.075 mg/mL DNA.

This experiment was performed in a high throughput small-scale model. The control, denoted as Condition B in Table 16, was the process denoted as DOE3 F1 in Table 37. The remainder of the conditions had the same parameters, but with varying levels of linearized plasmid DNA. RNA samples were purified by precipitation and concentration was measured by UV. Percent integrity was determined by Fragment Analyzer (FA).

Results from this experiment are displayed in Table 16. When DNA concentration was varied between 0.025 and 0.075 mg/mL, mRNA concentration was impacted. mRNA concentration increased significantly when DNA concentration was increased from 0.025 mg/mL to 0.05 mg/mL although increasing DNA concentration from 0.05 mg/ml to 0.075 mg/mL did not have as large of an effect on resulting mRNA concentration. Integrity remained within assay variability within this range, indicating no impact to integrity was observed.

TABLE 16
DNA OFAT Results

| | pDNA | Resulting mRNA | |
|---|---|---|---|
| | Concentration | Concentration | Integrity |
| Condition | (mg/mL) | (mg/mL) | (%) |
| A | 0.025 | 4.2 | 82 |
| B (DOE3 F1) | 0.05 | 6.5 | 83 |
| C | 0.075 | 7.1 | 80 |

### Example 13

### Effect of Mg and Total NTPS on IVT Performance

The ANOVA model shown in Table 17 contains a significant interaction term between Total NTPs and Mg (labeled AC). It was of interest to confirm the model. Additionally, further confirmation studies were desired to supplement the data. The goal of this experiment was to determine the impact of varying sum of ATP+CTP+GTP+pUTP from 28-44 mM. At each NTP level, four different Mg/NTP levels were assessed: 1.1, 1.3, 1.4, and 1.6. The experiment was performed in a small-scale high-throughput model with varying levels of NTPs and varying levels of Mg as shown in Table 17. All other parameters were taken from the condition DOE3 Opt-listed in Table 37. RNA samples were purified by precipitation and concentration was approximated by UV. Percent integrity was determined by Fragment Analyzer.

TABLE 17
Varied Parameters in Mg/NTP Experiment

| ATP + CTP + GTP + pUTP (mM) | Mg/NTP Ratio | Mg (mM) |
|---|---|---|
| 28 | 1.1 | 32 |
| 28 | 1.3 | 36 |
| 28 | 1.4 | 40 |
| 28 | 1.6 | 44 |
| 36 | 1.1 | 41 |
| 36 | 1.3 | 46 |
| 36 | 1.4 | 51 |
| 36 | 1.6 | 57 |
| 44 | 1.1 | 50 |
| 44 | 1.3 | 57 |
| 44 | 1.4 | 63 |
| 44 | 1.6 | 69 |

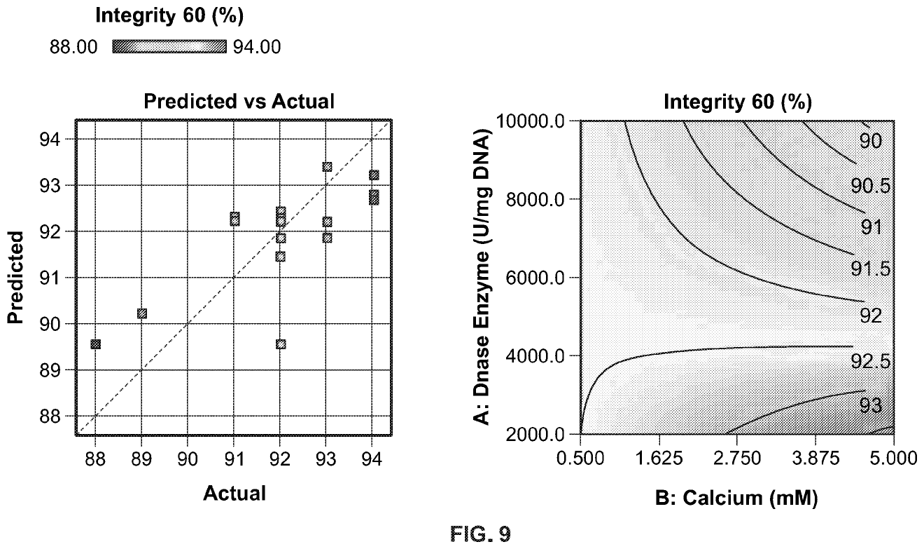

The impact of Mg concentration and total ATP+CTP+GTP+pUTP on IVT reaction performance is shown in FIGS. 9A-B. Adding NTPs above 28 mM increased RNA concentration considerably. However, it was observed that increasing Mg levels too high while keeping NTP concentration constant can also impact RNA concentration.

### Example 14

### Impact of Divalent Metals on Residual DNA

This DOE experiment was carried out to investigate the impact of divalent metals: calcium, magnesium and manganese during the DNA digestion step. Table 18 shows a four factorial DOE design with axial points. The range of calcium concentrations were obtained from a standard protocol (THERMO FISHER® DNase I) using a 1× concentration for the low and a 10× concentration for the high. The range of magnesium used a low and high of 1 mM and 10 mM, respectively, to account for loss of magnesium from pyrophosphate complexing. The low and high range of manganese mimicked the range of calcium. Time course samples were taken at 30 mins, 45 mins and 60 mins during DNase digestion.

Automated microbioreactor systems were used and Process FB V1 with DNase I digestion was used as a baseline. The reactions completed in this experiment used modified Process FB V1 with 0.05 U/mL pyrophosphatase concentration to determine residual DNA trends. Calcium, magnesium, manganese, and DNase enzyme concentrations were varied within DOE ranges and were added to DNase I enzyme addition. The concentrations studied for the DOE are provided in Table 18 below. Experimental outputs were RNA yield, integrity, and residual DNA.

TABLE 18
Varied Parameters and DOE Design

| Process | | | | Low | Center | High | One |
|---|---|---|---|---|---|---|---|
| Parameter | Name | Units | Target | (−1) | (0) | (+1) | Unit* |
| A | DNase | U/mg | 6000 | 2000 | 6000 | 10000 | 4000 |
| | Enzyme | DNA | | | | | |
| B | Calcium | mM | 2.75 | 0.5 | 2.75 | 5 | 2.25 |
| C | Magnesium | mM | 5.5 | 1 | 5.5 | 10 | 4.5 |
| D | Manganese | mM | 2.75 | 0.5 | 2.75 | 5 | 2.25 |

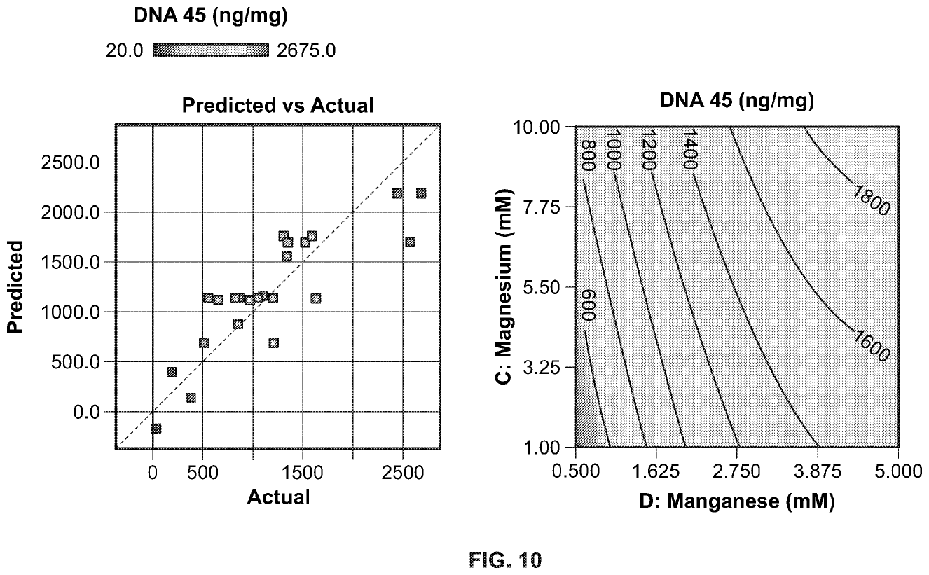

The results for this DOE design show that DNase I enzyme concentration and the calcium concentration ranges had no impact on residual DNA trends at the 30 minute and 45 minute time points, however have an impact on integrity at the 60 minute mark (FIG. 9). Manganese and magnesium show impact on residual DNA trends at both the 30 minute (data not shown) and 45 minute (FIG. 10) time point. The contour plot in FIG. 10 demonstrates that the low points for manganese and magnesium concentration resulted in the lowest residual DNA impurity.

### Example 15

### Proteinase K Analysis

After IVT is complete and prior to downstream purification, breaking down proteins with a protease such as Proteinase K (ProK) is ideal to further quench the IVT reaction and reduce risks of off-target behavior by proteins such as DNase I. Furthermore, the breakdown of larger proteins facilitates robust clearance by purification methods such as UF/DF, chromatography, etc. Proteinase K has been described as an enzyme that is commonly used to purify RNA [Tullis and Rubin, Anal Biochem 107 (1): 260-264 (1980)]. A bulk IVT reaction was assembled (“Conf Opt”) and subjected to DNase digestion with the setpoints identified described in Table 37 under the condition “Conf Opt.” DNase digestion parameters included 1 mM calcium chloride, 2000 DNase I (U/mg pDNA) for 45 minutes of DNase digestion time at a DNase digestion temperature of 36 C. This bulk mixture was separated into smaller aliquots and further digested with Proteinase K using the parameters described in Table 19. The recommended range of approximately 1.5-30 U/mL of ProK at 50° C. for the digestion step (THERMO FISHER® Proteinase K) was extended to a lower limit of 0.5 U/mL and modified to run for 45 minutes at the reaction temperature of 36° C. to prevent damage to the mRNA.

EDTA was used to reduce the levels of free CaCl₂) within the reaction prior to ProK digestion, as CaCl₂) can potentially inhibit ProK's ability to digest DNase [Tullis and Rubin, Anal Biochem 107 (1): 260-264 (1980)]. 50 mM EDTA was sufficient to quench the IVT reactions described in example 8, therefore 50 mM EDTA was used as the starting point for the experiment. After the incubation period, samples were flash frozen and submitted for the residual protein assay on the Perkin Elmer LabChip. Integrity was also measured by Fragment Analyzer (FA).

TABLE 19
ProK Digestion Conditions

| | | EDTA | ProK | | |
|---|---|---|---|---|---|
| | | Conc | Conc | Integrity | Residual |
| Condition | Process | (mM) | (U/mL) | (%) | Protein |
| A | Conf Opt | 50 | 30 | 84 | None detected |
| B | | 50 | 15 | 85 | None detected |
| C | | 50 | 1.5 | 85 | None detected |
| D | FB V1 | 75 | 1.5 | 92 | None detected |
| E | | 75 | 1.0 | 92 | None detected |
| F | | 75 | 0.5 | 94 | None detected |

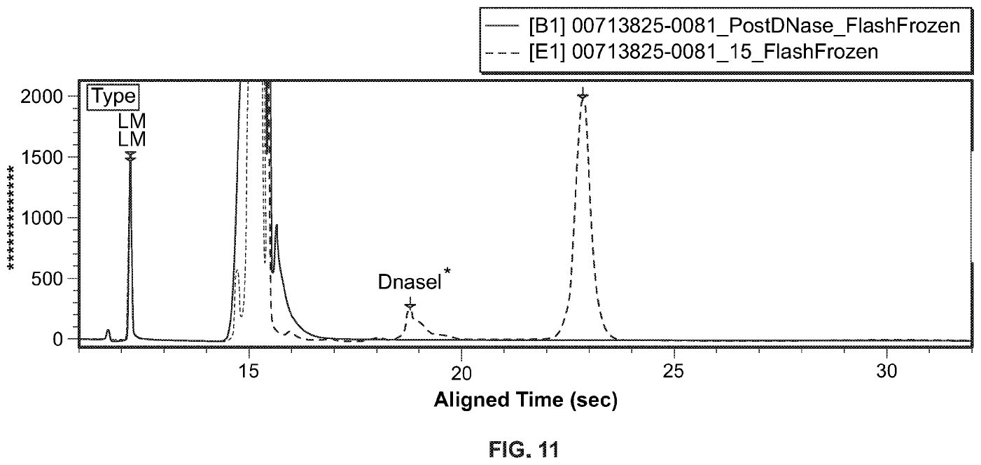

The integrity results are listed in Table 19. Integrity did not appear to be impacted by modulating levels of Proteinase K. Complete protein digestion was confirmed by the residual protein assay across all of the ProK conditions. The conditions for rows A, B, and C (“Conf Opt”) listed in Table 19 were run on the residual protein assay and the resulting electropherogram is shown in FIG. 11. The conditions for rows D, E, and F (“FB V1”) were obtained using the fed-batch process labeled FB V1 and described in Table 65. A sample that was not ProK digested was run as a control. This control contained peaks at around 19 seconds and 23 seconds that correspond to Dnase I and T7, respectively. For a sample dosed with 1.5 U/mL ProK, these peaks were no longer visible, which suggests that the digest was successful. Further, this demonstrates that ProK ranges of 1.5 U/mL and above are sufficient for digestion, with no impact on integrity at the range of concentrations tested. The residual protein analysis was also performed on the conditions for rows D, E, and F, and the resulting electropherogram showed similar results (data not shown), with the same DNase I and T7 peaks observed in the control sample at 19 seconds and 23 seconds respectively, but the samples dosed at 0.5, 1.0, and 1.5 U/mL ProK did not have any detected peaks at those residence times.

### Example 16

### General Applicability of IVT to Various RNA Constructs

The goal of this experiment was to evaluate whether the IVT batch process developed herein could be applicable to other RNA molecules of various sizes and compositions. To this end, RNA #2 was manufactured using Process BV1. The impact of spermidine on process performance was also evaluated. Data shown in Table 20 below demonstrates that Process BV1 manufactures high-quality RNA. Furthermore, as shown in Table 20, removing spermidine improves RNA capping efficiency, which further demonstrates that spermidine can be removed from the IVT process.

TABLE 20
General applicability of Process BV1 on RNA#2

| | Spermidine | DTT | Resulting RNA | Integrity | 5′Cap |
|---|---|---|---|---|---|
| Condition | (mM) | (mM) | Conc (mg/mL) | (%) | (%) |
| A | 0 | 0 | 8.77 | 93.2 | 91.1 |
| B | 2.15 | 0 | 8.98 | 91.5 | 86.6 |
| C | 0 | 20 | 9.62 | 92.7 | 91.4 |
| D | 2.15 | 20 | 8.96 | 90.9 | 86.5 |

Next, Process BV1-modified (outlined in Table 37) was evaluated using RNA #3, which is ˜5.7× larger than RNA #2. Process BV1-modified is modular and can easily be tailored to both enzymatic and co-transcriptional processes. Here, a co-transcriptional process was implemented using a capping molecule, although an analogue capping molecule could also be used (Table 21). RNA samples were purified by precipitation and IVT yield (as measured by UV), integrity (as measured by Fragment Analyzer), 5′ cap (as measured by LC-UV), and residual DNA (as measured by quantitative Polymerase Chain Reaction) were determined (Table 22). This example demonstrated high capping efficiency and RNA integrity using larger RNA constructs and the flexibility of Process BV1-modified in the manufacture of various RNAs.

TABLE 21
Experimental Parameters

| | Value Tested in | Value Tested in |
|---|---|---|
| Parameter Varied | 1 mL Reaction | 100 ml Reaction |
| Conc of 5′ capping analogue (mM) | 2-9 | 6 |
| Conc of Pyrophosphatase (U/mL) | 0.02-0.5 | 0.25 |
| IVT time (min) | 150-210 | 150 |
| Temperature (degrees C.) | 25-36 | 36 |

RNA yield was positively correlated to IVT temperature, and RNA yield was increased by ˜18-fold by increasing temperature to 36° C. Furthermore, all conditions produced sufficiently high RNA integrity of >70% using a large construct.

TABLE 22
Determination of IVT Temperature on IVT
Performance using Process BV1-modified

| | IVT time | IVT Temp | mRNA Yield | Integrity |
|---|---|---|---|---|
| Condition | (min) | (° C.) | (g/L ) | (%) |
| A | 150 | 36 | 7.1 | 71 |
| C | | 33 | 3.4 | 77 |
| E | | 30 | 1.5 | 74 |
| G | | 27 | 0.6 | 81 |
| | | 25 | 0.4 | 82 |

Next, impact of IVT time (150-210 min), and pyrophosphatase concentration (0.02-0.5 U/mL) were evaluated. As shown below in Table 23-Table 26, increasing IVT time results in >20% increase in RNA yield, however, a slight decrease in integrity was also observed. Furthermore, a few conditions were evaluated for capping efficiency and all of them were >90% demonstrating Process BV1-modified robustness in producing high-quality RNA.

TABLE 23
Effect of IVT time and 0.5 U/mL Pyrophosphatase
concentration on Reaction Performance

| | IVT time | IVT Temp | Pyrophosphatase | mRNA | | |
|---|---|---|---|---|---|---|
| Condition | (min) | (° C.) | (U/mL) | Yield (g/L) | Integrity (%) | 5′Cap (%) |
| A | 150 | 36 | 0.5 | 7.8 | 69 | N/A |

TABLE 24
Effect of IVT time and 0.25 U/mL Pyrophosphatase
concentration on Reaction Performance

| | IVT | IVT | Pyrophos- | mRNA | | |
|---|---|---|---|---|---|---|
| | time | Temp | phatase | Yield | Integrity | 5′Cap |
| Condition | (min) | (° C.) | (U/mL) | (g/L) | (%) | (%) |
| B | 150 | 36 | 0.25 | 7.7 | 68 | 94 |
| E | 210 | | | 9.2 | 63 | 93 |

TABLE 25
Effect of IVT time and 0.1 U/mL Pyrophosphatase
concentration on Reaction Performance

| | IVT | IVT | Pyrophos- | mRNA | | |
|---|---|---|---|---|---|---|
| | time | Temp | phatase | Yield | Integrity | 5′Cap |
| Condition | (min) | (° C.) | (U/mL) | (g/L) | (%) | (%) |
| H | 150 | 36 | 0.1 | 7.4 | 68 | N/A |
| K | 210 | | | 9.0 | 62 | N/A |

TABLE 26
Effect of IVT time and 0.02 U/mL Pyrophosphatase
concentration on Reaction Performance

| | IVT | IVT | Pyrophos- | mRNA | | |
|---|---|---|---|---|---|---|
| | time | Temp | phatase | Yield | Integrity | 5′Cap |
| Condition | (min) | (° C.) | (U/mL) | (g/L) | (%) | (%) |
| N | 150 | 36 | 0.02 | 5.3 | 69 | N/A |
| Q | 210 | | | 7.5 | 68 | 96 |

Capping molecule concentration was also evaluated. The concentration range of 2-9 mM was tested and as shown in Table 27, a dose-dependent response was observed between RNA concentration and capping molecule.

TABLE 27
Effect of Capping molecule concentration on Reaction Performance

| | IVT time | IVT Temp | Capping analogue | mRNA Yield |
|---|---|---|---|---|
| Condition | (min) | (° C.) | (mM) | (g/L ) |
| A | 150 | 36 | 2 | 3.1 |
| B | | | 4 | 5.8 |
| C | | | 6 | 8.2 |
| D | | | 7.2 | 8.3 |
| E | | | 9 | 7.2 |

To demonstrate Process BV1-modified is scalable, a 100 mL scale IVT run was conducted on an AMBR® 250 multi-parallel bioreactor system (SARTORIUS®). As shown in Table 28, data were comparable to 1 mL scale, and it also showed very low residual DNA impurities.

TABLE 28
Scale-up IVT of RNA#3 in AMBr 250 System

| | | | Residual DNA |
|---|---|---|---|
| Condition | mRNA Yield (g/L) | 5′Cap (%) | (ng/mg product) |
| A | 8.7 | 98 | <1 |

To further improve mRNA integrity, mRNA affinity chromatography steps were developed using POROS™ oligo (dT) 25 (THERMO FISHER®) and oligo (dT) 18 monolith (SARTORIUS BIA SEPARATIONS®) purification. Both steps bound mRNA in 10 mM Tris/500 mM KCl/0.1 mM EDTA, pH 7.2 and were eluted with 10 mM Tris/0.1 mM EDTA, PH 7.2. As shown in Table 29 below, mRNA integrity improved from 61% for non-affinity purified material up to 79% when purified by the affinity step.

TABLE 29
mRNA Affinity Chromatography

| Condition | Pool Integrity (%) |
|---|---|
| Non-Affinity Purified Pool | 61 |
| POROS oligo(dT)25 Elution Pool | 76 |
| Sartorius BIA Separations oligo(dT)18 | 79 |
| Monolith Elution Pool | |

The data in this example demonstrated the general applicability and scalability of Process BV1 with multiple RNA constructs of different sizes. This process was applied to RNA #3 and demonstrated comparable product quality and low impurities.

### Example 17

### Development of a Fed-Batch Process

The goal of this experiment was to develop a fed-batch process by feeding through boluses, semi-continuous, and or continuous manner [Kern and Davis, Biotechnol Prog 13 (6): 747-56 (1997)] starting from Process BV1 described above. Here, automated microbioreactor systems were used to determine feeding approaches. Experimental design is shown in Table 30 below and initial baseline NTPs were adapted from Henderson et al. [Henderson et al., Curr Protoc 1 (2): e39 (2021)]. Furthermore, an approach using various initial GTP concentrations was used [mMESSAGE mMACHINE® Kit User Guide (Pub. No. 1340M Rev. G) THERMO FISHER] to evaluate impact on capping efficiency while maintaining total concentrations through feeds [Kern and Davis, Biotechnol Prog 13 (6): 747-56 (1997)].

TABLE 30
Outline of Fed-Batch Evaluation Experiment

| | | Total | Initial | Initial | Bolus |
|---|---|---|---|---|---|
| | | NTPs | NTPs | GTP | Interval |
| Condition | Process | (mM) | (mM) | (mM) | (min) |
| A | BV1 | | 9 | 9 | N/A |
| B | Fed Batch | 9 mM each | 5 | 5 | 5 |
| C | | | | | 10 |
| D | | | | | 25 |
| E | | | | 2.5 | 5 |
| F | | | | | 10 |
| G | | | | | 25 |
| H | | | | 1 | 5 |
| I | | | | | 10 |
| J | | | | | 25 |

Automated microbioreactor systems were used and Process BV1 was modified by reducing pyrophosphatase concentration and increasing temperature to 0.25 U/mL and 37° C., respectively. This modified Process BV1 was used as a baseline in these experiments. Experimental outputs were RNA yield, integrity, capping efficiency, and residual NTPs.

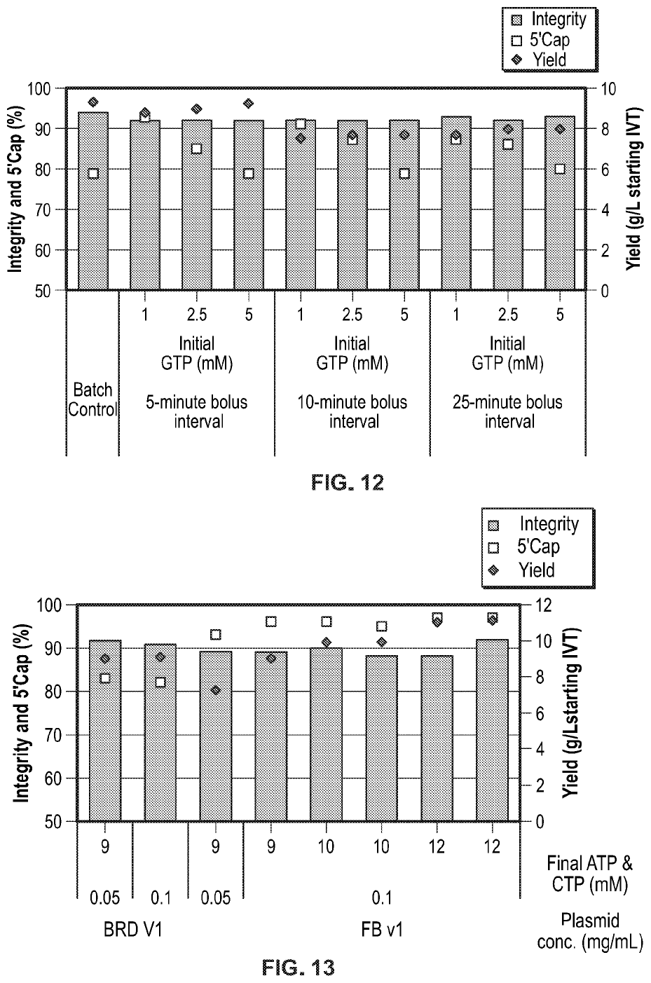

As shown in FIG. 12, RNA integrity was comparable across all conditions. However, a high frequency NTPs feeding schedule at 5 min intervals had better yield compared to its 10 min and 25 min counterparts. Furthermore, decreasing initial GTP concentration increased capping efficiency across all feeding schedules (highest at 93%). Fed-batch process with GTP concentrations at 1 and 2.5 mM had better capping efficiency than batch process. Next, residual NTPs after 150 min of IVT were evaluated and some NTPs were low (data not shown). To this end, a follow up experiment was completed to evaluate increased NTP concentrations.

BV1 Process was used as a baseline control in this second fed-batch development experiment. The best fed-batch process (highest capping efficiency at 93%, condition H in Table 30 above) was further used in the follow-up experiment by moderately adjusting NTP concentrations to similar ranges described in FIGS. 9A-9B and DNA template concentration to expand upon the results from Example 12: linearized plasmid DNA (pDNA), which demonstrated improved yield with increased pDNA concentration. The experimental design is shown in Table 31 below.

TABLE 31
Experiment 2 Reaction Conditions Evaluating
Higher NTPs and DNA concentration

| | DNA concentration | Final ATP & CTP | Final GTP |
|---|---|---|---|
| Process | (mg/ml) | (mM) | (mM) |
| Process BV1 | 0.05 | 9 | 9 |
| | 0.1 | | 9 |
| Condition H | 0.05 | 9 | 9 |
| | 0.1 | 9 | 9 |
| | | 10 | 9 |
| | | 10 | 10 |
| | | 12 | 9 |
| | | 12 | 10 |

Results of this experiment are shown in FIG. 13. Yield and % 5′-cap improved to 11.1 mg/mL and 97% respectively when increasing ATP/CTP to 12 mM and the final GTP concentration to 10 mM. Therefore, Condition H with 0.1 g/L plasmid DNA, 12 mM final ATP and CTP, and 10 mM final GTP (listed in Table 31) was continued forward for further development as Process FB V1.

### Example 18

### Scale-Up, Continuous Feed Simulation, and Synthetic DNA

The goals of this experiment were to demonstrate the scalability of Process FB V1 and that bolus feeds or semi-continuous feeds developed herein is technically equivalent to a continuous feed by producing comparable high quality RNA in Process FB V1. A further goal was to demonstrate that Process FB V1 is generally applicable in that it produces high quality RNA (using either pDNA or synthetically produced DNA, for example, from a unified sequential template amplification and transcription [USTAT] process) that is comparable across scales. NTP feeds were implemented either through bolus or semi-continuous feeds and continuous feeds using automated microbioreactor systems.

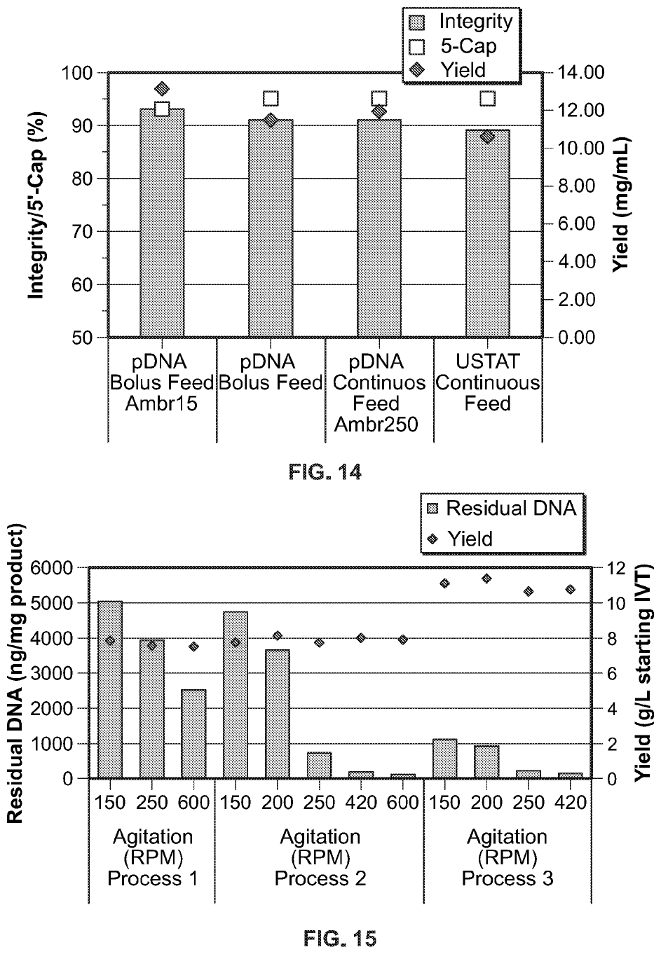

As shown in FIG. 14, bolus feeds or semi-continuous feeds produce RNA quality and yield that are comparable to a continuous feed counterpart. Data also show that the continuous feed process manufactures RNA with high and comparable quality using either pDNA or synthetically produced DNA (USTAT). Furthermore, Process FB V1 is also scalable across multiple scales as shown in FIG. 14. Together, these data show general scalability and applicability of Process FB V1 to various DNA template sources.

### Example 19

### Implementation of Mg Boluses in the IVT Reaction

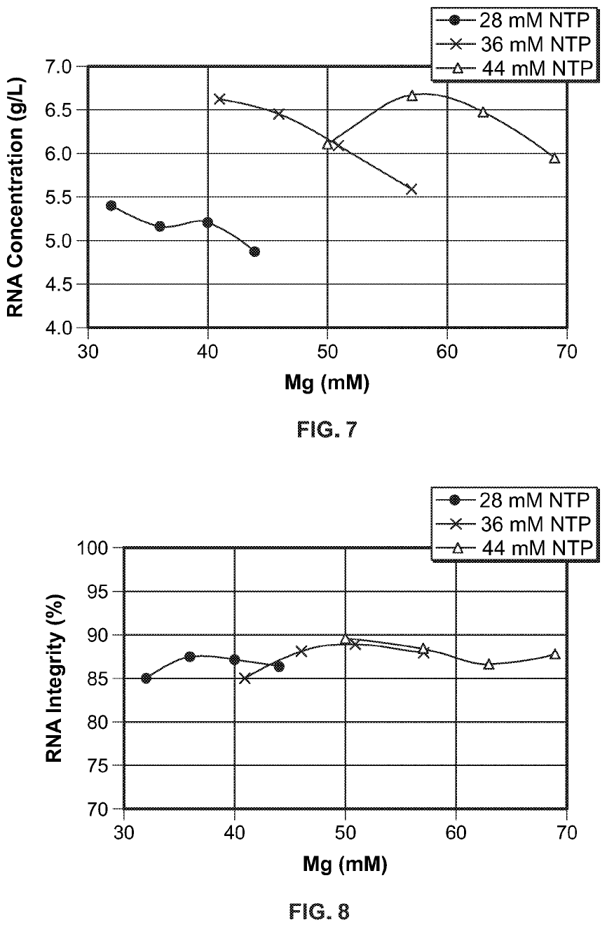

As shown in FIG. 7, either too high or too low levels of Mg impacts RNA concentration. Importantly, not only do the data show magnesium concentration has an optimal operating range, but also addition strategy during IVT can be optimized.

As more NTPs are added to the reaction system over time, the Mg:NTP ratio may not remain consistent. Further, precipitates of Mg2+ and pyrophosphate that form throughout the reaction can lower the amount of Mg available to enzymes such as T7 as shown in FIG. 17 and reported elsewhere [[Kartje et al., J Biol Chem 296 (2021)]. For these reasons, gradual delivery of Mg was explored in order to improve RNA quality attributes without negatively impacting yield and maintain a more consistent ratio of Mg:NTPs throughout the reaction.

Reaction systems with gradual feeding of a mixture of NTP and MgCl₂, where three feeding phases were initiated based on in-line measurements of residual NTPs from CIMac PrimaS™ HPLC, have been described [Skok et al., Chem Ing Tech 94 (12): 1-9 (2022)]. Unlike those systems previously described, the process described herein is not divided into multiple feeding phases and does not rely on measurements from a CIMac PrimaS™ HPLC system. Additionally, the feed consists of MgAcetate and further optimized levels of NTPs, demonstrating an overall benefit to % capping.

The IVT reactions were assembled with varying starting levels of ATP, CTP, GTP, and pUTP and Mg acetate. Throughout the reaction, a combined feed of ATP, CTP, GTP, and pUTP was used to reach the total levels listed in Table 32. Some conditions were fed with boluses of Mg acetate every 35 minutes while other conditions were “front loaded” with the target amount of Mg acetate. Samples were precipitated prior to analytical measurements. Concentration was measured by UV, integrity was measured by Fragment Analyzer (FA), and % cap was measured by an LC-UV method.

As shown in Table 32, the conditions that were front loaded with Mg had lower % capping than their counterparts that received Mg boluses. In one pair of conditions, Mg boluses appeared to improve capping by approximately 15%. Each pair had comparable mRNA yields within experimental and analytical variability, but this is likely because the Mg:NTP ratio was kept consistent across all tested conditions.

TABLE 32
Effect of Mg Boluses on IVT Performance

| Starting / | | Starting / | | | | | | |
|---|---|---|---|---|---|---|---|---|
| Ending | Starting / | Ending | Starting | Ending | Mg | mRNA | | |
| ATP and | Ending | pUTP | Mg | Mg | Delivery | Yield (g/L | Integrity | 5′Cap |
| CTP (mM) | GTP (mM) | (mM) | (mM) | (mM) | Method | Starting) | (%) | (%) |
| 7/12 | 3/12 | 7/11 | 43 | 143 | Front- | 6.8 | 80 | 66 |
| | | | | | Loaded | | | |
| 7/12 | 3/12 | 7/11 | 36 | 43 | Bolus | 7.2 | 81 | 72 |
| 9/14 | 5/14 | 9/13 | 50 | 50 | Front- | 7.9 | 78 | 56 |
| | | | | | Loaded | | | |
| 9/14 | 5/14 | 9/13 | 36 | 50 | Bolus | 8.5 | 80 | 66 |
| 11/16 | 7/16 | 11/15 | 57 | 57 | Front- | 8.8 | 81 | 52 |
| | | | | | Loaded | | | |
| 11/16 | 7/16 | 11/15 | 36 | 57 | Bolus | 8.7 | 80 | 67 |

This example demonstrates the benefits to gradually feeding Mg. For instance, in this example, gradual feeding of Mg improved 5′ cap, demonstrating that gradual delivery of Mg is a strategy to improve capping. The Mg bolus approach may also demonstrate at varying Mg:NTP target ratios that gradual feeding is needed to maintain yield.

### Example 20

### Capping Molecule Concentration and Scale-Up

This example explored the feasibility of decreasing capping molecule concentration down to 2.5 mM while still maintaining 95% capping. As shown in Example 19: Implementation of Mg Boluses in IVT Reaction, gradual feeding of Mg may boost capping. For this reason, starting Mg was also investigated in this example while ending Mg was kept constant at 36 mM. An additional objective was to determine whether the Mg feeding strategy was scalable and could be performed through a continuous feed.

This example includes two separate experiments. One was performed in a 15 mL reactor while the other experiment was performed in a 100 mL reactor. The constant parameters in each experiment are listed in Table 38 and the varying parameters are shown in Table 33. In the 8 mL system, a feed of ATP/CTP/GTP/modified UTP was delivered every 5 minutes and a separate feed of MgAc was delivered every 15 minutes. In the 90 mL system, a combined feed of NTPs and MgAc was fed continuously. Concentration was measured by a UV method, integrity was measured by Fragment Analyzer (FA), and % capping was evaluated by an LC-UV assay.

As shown in Table 33, all reactions at the 8 mL scale performed well. mRNA yield and Fragment Analyzer (FA) integrity across all tested conditions was within assay and analytical variability. A marginal increase of approximately 3% in capping was observed between condition B and F, which is only slightly above the expected assay variability. Because the 5′cap was so high across the board, Mg bolusing did not contribute as much to % cap, but it may still assist in overall process robustness if GTP is overfed. Based on these results, it may be feasible to reduce capping molecule concentration below 2.5 mM.

TABLE 33
Capping Molecule and Mg Assessment 8 mL

| | Capping | Starting | Ending | mRNA | FA | |
|---|---|---|---|---|---|---|
| Condi- | Molecule | Mg | Mg | Yield (g/L | Integrity | 5′Cap |
| tion | Conc (mM) | (mM) | (mM) | Starting) | (%) | (%) |
| A | 4 | 36 | 36 | 10.6 | 91 | 96 |
| B | 2.5 | 36 | 36 | 10.7 | 91 | 94 |
| C | 4 | 24 | 36 | 10.3 | 93 | 97 |
| D | 2.5 | 24 | 36 | 10.2 | 93 | 95 |
| E | 4 | 18 | 36 | 10.6 | 93 | 98 |
| F | 2.5 | 18 | 36 | 10.5 | 93 | 97 |

The reaction concentration and integrity from Condition B was compared to the performance of the 90 mL condition (Table 34). RNA concentration and integrity were within experimental variability, which suggests that the process can be scaled up and a combined Mg:NTP feed is feasible to simplify operations. It also provides support that Mg boluses are comparable to a continuous feed containing Mg.

TABLE 34
Comparison of AMBr15 to EasyMax Performance

| Starting | NTP and Mg Feed | RNA Concentration | Integrity |
|---|---|---|---|
| Scale | Delivery Strategies | (mg/ml) | (%) |
| 8 mL | NTP bolus delivered | 6.8 | 93 |
| | every 5 minutes. Mg | | |
| | feed delivered every | | |
| | 15 minutes. | | |
| 90 mL | Single feed composed of | 7.7 | 92 |
| | NTPs and Mg delivered | | |
| | continuously throughout | | |
| | reaction. | | |

As demonstrated herein, capping molecule can be used at 2.5 mM (and possibly lower) and the process can be run with 5-min boluses, with a continuous feed, with separate Mg and NTP feeds, and/or combined Mg/NTP feeds.

### Example 21

### High NTP Feeding

This example explored the highest level of NTPs that can be fed at 36 mM front-loaded Mg acetate. In Example 10, an interaction term between NTPs and Mg was observed. For this reason, it was of interest to determine how robust the process is over a wide variety of NTP concentrations.

This example was performed at an 8 mL starting volume in an automated microscale bioreactor system. All constant parameters are listed in Table 38. A liquid handler was used to add varying amounts of feed that contained ATP, CTP, GTP, and pUTP every 5 minutes to reach the total levels listed in Table 35. Concentration was measured by UV, integrity was measured by FA, and % capping was evaluated by an LC-UV assay.

The process showed robustness over a wide range of NTP concentrations. Every reaction produced a significant amount of mRNA at a range of 45-79 mM total NTPs. In Example 10, the batch process showed a lower optimal range of NTPs. Together, these results suggest that a fed batch process can handle higher total NTPs with high yield and improved product quality profiles.

TABLE 35
High NTP Feeding Results

| | Start | Total | Start | Total | Start | Total | mRNA | |
|---|---|---|---|---|---|---|---|---|
| | ATP/CTP | ATP/CTP | GTP | GTP | pUTP | pUTP | Yield (g/L | Integrity |
| Condition | (mM) | (mM) | (mM) | (mM) | (mM) | (mM) | Starting) | (%) |
| A | 5 | 12 | 1 | 10 | 5 | 9 | 10.3 | 91 |
| B | 5 | 15 | 1 | 14 | 5 | 11 | 12.4 | 92 |
| C | 5 | 18 | 1 | 18 | 5 | 12 | 14.5 | 92 |
| D | 7 | 14 | 1.4 | 10 | 7 | 11 | 11.8 | 91 |
| E | 7 | 17 | 1.4 | 14 | 7 | 13 | 13.8 | 93 |
| F | 7 | 20 | 1.4 | 18 | 7 | 14 | 14.6 | 93 |
| G | 9 | 16 | 1.8 | 10 | 9 | 13 | 11.0 | 93 |
| H | 9 | 19 | 1.8 | 14 | 9 | 15 | 12.4 | 92 |
| I | 9 | 22 | 1.8 | 18 | 9 | 16 | 13.3 | 93 |

### Example 22

### Impact of Agitation on Residual DNA

Impurities in a drug substance need to be well controlled and minimized for patient safety. One of the impurities that is challenging to remove from the IVT process is residual DNA (resDNA). A number of parameters were investigated with regard to their effect on resDNA. Here, agitation and mixing parameters were identified and shown as key factors that impact resDNA.

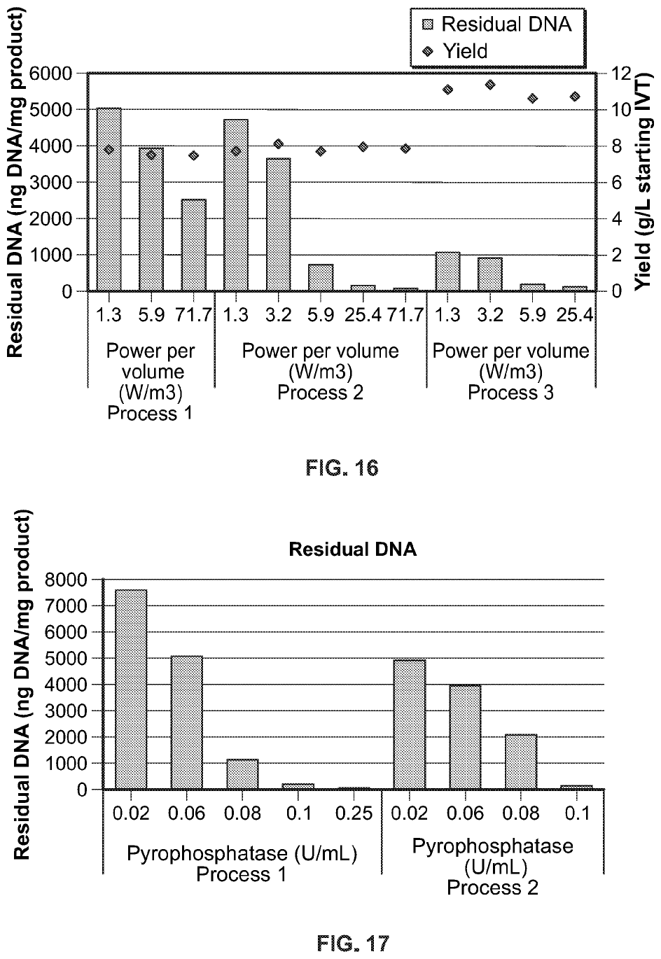

Three processes were performed in this experiment with RNA #1 in an automated microbioreactor system. Process FB V1 with DNase digestion was used as a baseline to investigate the impact of mixing parameters on resDNA impurity. These reactions were modified from Process FB V1 in agitation rate and pyrophosphatase concentrations. The three processes shown in FIG. 15 and FIG. 16 evaluated the agitation ranges described below at three varying pyrophosphatase levels across multiple vendors. The agitation rates studied ranged from 150-600 RPM (FIG. 15) which is synonymous to ranges of power per volume (FIG. 16), mixing time, and impeller tip speeds. These ranges are listed below in Table 36. These four measures of mixing, as well as other mixing factors not listed, can be used similarly to reduce residual DNA impurity levels.

TABLE 36
Studied Mixing Parameter Ranges

| Agitation | Power/Volume | Mixing Time | Impeller Tip Speed |
|---|---|---|---|
| (RPM) | (W/m³) | (S) | (m/s) |
| 150-600 | 1.3-71.7 | 1.3-12.1 | 0.1-0.4 |

The results of this experiment highlight the impact of agitation rate on residual DNA impurities, as residual DNA levels are reduced consistently across all three processes as agitation rate, or power per volume, mixing time, or impeller tip speed, is increased. Agitation rate can therefore be modulated intentionally to reduce residual DNA impurities in the IVT reaction. This example demonstrates that the agitation rate range studied corresponds to multiple parameters that affects the mixing profile, including parameters such as power per volume, impeller tip speed, and mixing time, and that these mixing parameters can be modulated similarly to reduce resDNA impurity.

TABLE 37
Batch Process Parameters

| | Setpoint | | | | | | | |
|---|---|---|---|---|---|---|---|---|
| | Example | Example | Example | DOE3 | DOE3 | Conf | | |
| Parameter | 8: DOE1 | 9: DOE2 | 10: DOE3 | F1 | Opt | Opt | BV1 | BV1-modified |
| 5′Cap analog | 4 | 4 | 4 | 4 | 4 | 9 | 4 | See Table 21 in |
| molecule (mM) | | | | | | | | Example 16 |
| ATP (mM) | 5 | N/A | N/A | 9 | 7 | 9 | 9 | 9 |
| CTP (mM) | 5 | N/A | N/A | 9 | 7 | 9 | 9 | 9 |
| GTP (mM) | 5 | N/A | N/A | 9 | 7 | 9 | 9 | 9 |
| Modified UTP | 5 | N/A | N/A | 9 | 7 | 9 | 9 | 9 (UTP) |
| (mM) | | | | | | | | |
| Tris Concentration | N/A | 40 | 40 | 40 | 40 | 40 | 40 | 40 |
| (mM) | | | | | | | | |
| Tris Buffer pH | 8.0 | N/A | 8.0 | 8.0 | 8.0 | 8.0 | 8.0 | 8.0 |
| Magnesium (mM) | 16.5 | N/A | N/A | 36 | 33 | 50 | 36 | 36 |
| Spermidine (mM) | N/A | 2 | 0 | 0 | 0 | 0 | 0 | 0 |
| DTT (mM) | N/A | 10 | 0 | 0 | 0 | 0 | 0 | 0 |
| Triton (%) | N/A | 0 | 0 | 0 | 0 | 0 | 0 | 0 |
| DMSO (%) | N/A | 0 | 0 | 0 | 0 | 0 | 0 | 0 |
| PEG-8000 (%) | N/A | 0 | 0 | 0 | 0 | 0 | 0 | 0 |
| DNA Conc | 0.05 | 0.05 | 0.05 | 0.05 | 0.05 | 0.075 | 0.05 | 0.05 |
| (mg/mL) | | | | | | | | |
| Rnase Inhibitor | 1000 | 1000 | 100 | 100 | 100 | 100 | 100 | 100 |
| (U/mL) | | | | | | | | |
| Pyrophosphatase | 2 | N/A | 0.5 | 0.5 | 0.5 | 0.5 | 0.25 | See Table 21 in |
| (U/mL) | | | | | | | | Example 16 |
| T7 Polymerase | 8000 | N/A | N/A | 14000 | 17000 | 25000 | 14000 | 14000 |
| (U/mL) | | | | | | | | |
| Total IVT Time | 120 | N/A | 150 | 150 | 150 | 150 | 150 | See Table 21 in |
| (min) | | | | | | | | Example 16 |
| Temperature | 37 | N/A | N/A | 37 | 36 | 36 | 37 | See Table 21 in |
| (deg C.) | | | | | | | | Example 16 |

TABLE 38
Fed Batch Process Parameters

| | Setpoint | | | | |
|---|---|---|---|---|---|
| | | Example 19: | | | |
| | | Implementation | Example 20: | Example 20: | Example 21: |
| Parameter | | of Mg Boluses in | Concentration, | Concentration, | High NTP |
| Process Name | FB V1 | IVT Reaction | Scale-Up: 8 mL | Scale-Up: 90 mL | Feeding |
| 5′Cap analog | 4 | 2.5 | 2.5 | 2.5 | 2.5 |
| molecule (mM) | | | | | |
| Initial ATP (mM) | 5 | N/A | 5 | 5 | N/A |
| Final ATP (mM) | 12 | N/A | 12 | 12 | N/A |
| Initial CTP (mM) | 5 | N/A | 5 | 5 | N/A |
| Final CTP (mM) | 12 | N/A | 12 | 12 | N/A |
| Initial GTP (mM) | 1 | N/A | 1 | 1 | N/A |
| Final GTP (mM) | 10 | N/A | 10 | 10 | N/A |
| Initial modified | 5 | N/A | 5 | 5 | N/A |
| UTP (mM) | | | | | |
| Final modified | 9 | N/A | 9 | 9 | N/A |
| UTP (mM) | | | | | |
| NTP Delivery | Varies | Boluses | Boluses | Combined | Boluses |
| Strategy | | every 5 min | every 5 min | continuous | every 5 min |
| | | | | Mg/NTP feed | |
| Tris Buffer, pH | 40 | 40 | 40 | 40 | 40 |
| 8.0 (mM) | | | | | |
| HEPES Buffer, pH | 0 | 0 | 0 | 0 | 0 |
| 8.0 (mM) | | | | | |
| Starting Mg (mM) | 36 | N/A | N/A | 18 | 36 |
| Ending Mg (mM) | 36 | N/A | 36 | 36 | 36 |
| Mg Delivery | N/A | Separate bolus | Separate bolus | Combined | N/A |
| Strategy | | every 35 min | every 15 min | continuous | |
| | | | | Mg/NTP feed | |
| Spermidine (mM) | 0 | 0 | 0 | 0 | 0 |
| DTT (mM) | 0 | 0 | 0 | 0 | 0 |
| Plasmid DNA | 0.1 | 0.05 | 0.1 | 0.1 | 0.1 |
| Template | | | | | |
| (mg/ml) | | | | | |
| Rnase Inhibitor | 100 | 100 | 100 | 100 | 100 |
| (U/mL) | | | | | |
| Pyrophosphatase | 0.25 | 0.25 | 0.25 | 0.25 | 0.25 |
| (U/mL) | | | | | |
| T7 Polymerase | 14000 | 14000 | 14000 | 14000 | 14000 |
| (U/mL) | | | | | |
| Total IVT Time | 150 | 150 | 150 | 150 | 150 |
| (min) | | | | | |
| Temperature | 37 | 37 | 37 | 37 | 37 |
| (deg C.) | | | | | |

### Example 23

### Plasmids

Several plasmids were used in the following examples. The GFP plasmid (5854 bp) included the coding sequence for GFP (Green fluorescent protein) and a BsaI restriction endonuclease recognition site immediately downstream of the poly(A) tail sequence. The self-amplifying (SA) plasmid (13,826 bp) included the NSPs and RSV F protein and a LguI restriction endonuclease recognition site immediately downstream of the poly(A) tail sequence. The B globin plasmid (5101 bp) encoded for RSV F protein and a LguI restriction endonuclease recognition site immediately downstream of the poly(A) tail sequence tract. Both the GFP plasmid and the B globin plasmid used the 5′ and 3′ UTR of beta-globin. The poly(A) tail for the GFP plasmid RNA was 96 A's (segmented) and the poly(A) tail for the B globin and SA plasmid was 80 A's. E. coli strains harboring the desired plasmids were grown in animal-free LB broth with Kanamycin and the resulting cell paste was processed with a Plasmid Giga Kit (QIAGEN®) to isolate and purify the plasmid DNA. Plasmid DNA homogeneity was confirmed by Agarose gel electrophoresis.

Plasmid Linearization was done at 37 deg C. with 18 hours of Incubation. Plasmid GFP was linearized with 1.5U/μg BsaI restriction enzyme, SA plasmid was linearized with 0.5U/μg of LguI, and B globin plasmid was linearized with 1.5U/μg of LguI. Linearization was confirmed using agarose gel electrophoresis.

All in vitro transcription reaction components (except for the ribolock, pyrophosphatase, and T7 RNA polymerase) were thawed on ice for 10-15 minutes, the reaction components combined, and the reaction incubated for 2 or 3 hours. Following reaction completion, the DNA template was removed by treating with 6U/uL or 8U/ul DNase I and 1-2 mM CaCl₂) for 30 minutes. T7 RNA polymerase and pyrophosphatase (both THERMO FISHER SCIENTIFIC®) were used in the reactions. ATP, CTP, UTP, N1 methyl pseudo uridine (P-UTP) and GTP (THERMO FISHER SCIENTIFIC®) were stored at recommended temperature and used as needed. DTT and spermidine (both SIGMA ALDRICH®) were used as needed.

RNA was precipitated overnight at −80 deg C. in a freezer by lithium chloride. The pellet was then washed three times with chilled 100% ethanol followed by addition of nuclease free water to dry the pellet. RNA quantification and yield was calculated as mg/mL IVT reaction (NANODROP TECHNOLOGIES®).

### Example 24

### NTP Concentrations

Different concentrations of NTPs, e.g., 5 mM (total 20 mM NTP), 6 mM (total 24 mM NTP), 7 mM (total 28 mM NTP) and 8 mM (total 32 mM NTP) in the IVT reaction were studied to cover a broader range of NTPs. Cap analogue was added upfront in the reaction to produce capped mRNA in this Batch Mode IVT method development. The IVT Method described by Henderson et al. [Henderson et al., Curr Protoc 1 (2): e39 (2021)] has 5 mM of each NTP and 4 mM CleancapAG (30Me). For varied NTP concentrations, the individual NTP/CleancapAG (30Me) ratio was maintained at 1.25. The optimum concentration of magnesium, the cofactor of T7 RNA polymerase, can be determined to maintain ≥90% RNA Integrity and ≥90% cap incorporation with maximum achievable yield. Another hypothesis tested was whether Mg+2 concentration should be 4-6 mM more than total NTP. Therefore, varied concentrations of MgOAC2 were studied to find baseline concentration that worked comparable across different NTP concentrations. During the IVT reaction, as NTPs are incorporated by T7 RNA polymerase, pyrophosphate is released which forms a complex with magnesium and precipitates in reaction [Kern & Davis, Biotech progress 13 (6): 747-756 (1997). Pyrophosphatase enzyme can be added in the reaction to break pyrophosphate into monophosphate to make magnesium free and available in the reaction. Some groups have demonstrated pyrophosphatase in the IVT reaction increases RNA yield [Cunningham & Ofengand, Biotechniques 9 (6): 713-714 (1990)] whereas other groups have shown no effect of pyrophosphatase in increasing the RNA yield [Samnuan et al., F1000Research 11:333 (2022)]. Given these different observations, this example was tested with lower concentrations of pyrophosphatase to study broader ranges. To get maximum RNA yield from different IVT conditions, GFP template concentration was kept at 0.1 ug/uL. The range of T7 RNA polymerase concentration studied was 8U/uL and 10U/uL and the reactions have 1U/ul of Ribolock RNase inhibitor (THERMO FISHER SCIENTIFIC®), 10 mM DTT, 2 mM spermidine, and a reaction incubation temperature of 37 deg C., as shown in Table 39. The yield, % capped, and % RNA integrity from the reactions are shown in Table 40.

TABLE 39
IVT Process Parameters

| | Concen- | | | | |
|---|---|---|---|---|---|
| IVT Components | tration | Final concentration in the reaction | | | |
| Tris HCL buffer pH8 | mM | 40 | 40 | 40 | 40 |
| Each NTP | mM | 5 | 6 | 7 | 8 |
| Cleancap | mM | 4 | 4.8 | 5.6 | 6.4 |
| AG(3OMe) | | | | | |
| Ribolock | U/uL | 1 | 1 | 1 | 1 |
| Total NTP in | mM | 20 | 24 | 28 | 32 |
| reaction | | | | | |
| Magnesium | mM | 24, 26, | 28, 30, | 30, 32, | 30, 32, |
| | | 28, 30, | 32 | 34, 36 | 34, 36, |
| | | 32 | | | 38 |
| DTT | mM | 10 | 10 | 10 | 10 |
| Spermidine | mM | 2 | 2 | 2 | 2 |
| Pyrophosphatase, | mU/uL | 0.02 | 0.02 | 0.02 | 0.02 |
| Animal Origin-Free | | | | | |
| (AOF) | | | | | |
| T7 RNA | U/uL | 8, 10 | 8, 10 | 8, 10 | 8, 10 |
| Polymerase, AOF | | | | | |
| Temperature | DegC | 37 | 37 | 37 | 37 |
| Incubation Time | minutes | 120 | 120 | 120 | 120 |

TABLE 40
IVT Process Results

| | | | | | mM Mg + 2 | | | |
|---|---|---|---|---|---|---|---|---|
| | Cleancap | MgO | Each | T7 RNA | as | | | |
| Sample | AG(3OMe) | AC2 | NTP | polymerase | compared | Yield | % | % RNA |
| ID | mM | mM | mM | U/uL | total NTP | (mg/mL) | Cap | Integrity |
| 1 | 6.4 | 30 | 8 | 8 | −2 | 7.11 | 99 | 91.5 |
| 2 | 6.4 | 32 | 8 | 8 | 0 | 7.36 | 99 | 93.5 |
| 3 | 6.4 | 34 | 8 | 8 | 2 | 7.28 | 99 | 93 |
| 4 | 6.4 | 36 | 8 | 8 | 4 | 7.49 | 10 | 94.3 |
| | | | | | | | 0 | |
| 5 | 6.4 | 38 | 8 | 8 | 6 | 7.00 | 98 | 94.2 |
| 6 | 6.4 | 30 | 8 | 10 | −2 | 8.27 | 99 | 93.7 |
| 7 | 6.4 | 32 | 8 | 10 | 0 | 8.05 | 99 | 93.7 |
| 8 | 6.4 | 34 | 8 | 10 | 2 | 7.91 | 98 | 94.2 |
| 9 | 6.4 | 36 | 8 | 10 | 4 | 8.24 | 99 | 95 |
| 10 | 6.4 | 38 | 8 | 10 | 6 | 7.46 | 99 | 94.9 |
| 11 | 5.6 | 30 | 7 | 8 | 2 | 6.69 | 98 | 93.8 |
| 12 | 5.6 | 32 | 7 | 8 | 4 | 7.15 | 98 | 94.2 |
| 13 | 5.6 | 34 | 7 | 8 | 6 | 6.54 | 98 | 93 |
| 14 | 5.6 | 36 | 7 | 8 | 8 | 7.84 | 98 | 94.8 |
| 15 | 5.6 | 30 | 7 | 10 | 2 | 7.25 | 98 | 94.1 |
| 16 | 5.6 | 32 | 7 | 10 | 4 | 7.30 | 98 | 94.8 |
| 17 | 5.6 | 34 | 7 | 10 | 6 | 6.96 | 97 | 94 |
| 18 | 5.6 | 36 | 7 | 10 | 8 | 6.61 | 98 | 94.3 |
| 19 | 4.8 | 28 | 6 | 8 | 4 | 6.33 | 96 | 92.1 |
| 20 | 4.8 | 30 | 6 | 8 | 6 | 5.85 | 96 | 94.3 |
| 21 | 4.8 | 32 | 6 | 8 | 8 | 5.88 | 96 | 94.6 |
| 22 | 4.8 | 28 | 6 | 10 | 4 | 6.50 | 98 | 93.9 |
| 23 | 4.8 | 30 | 6 | 10 | 6 | 5.73 | 89 | 93.7 |
| 24 | 4.8 | 32 | 6 | 10 | 8 | 5.83 | 94 | 92.9 |
| 25 | 4 | 24 | 5 | 8 | 4 | 5.51 | 97 | 93.1 |
| 26 | 4 | 26 | 5 | 8 | 6 | 4.97 | 91 | 91.8 |
| 27 | 4 | 28 | 5 | 8 | 8 | 4.86 | 95 | 92.6 |
| 28 | 4 | 30 | 5 | 8 | 10 | 4.84 | 80 | 90.5 |
| 29 | 4 | 32 | 5 | 8 | 12 | 5.83 | 93 | 89.5 |
| 30 | 4 | 24 | 5 | 10 | 4 | 4.94 | 97 | 91.4 |
| 31 | 4 | 26 | 5 | 10 | 6 | 4.49 | 95 | 91.9 |
| 32 | 4 | 28 | 5 | 10 | 8 | 4.14 | 88 | 87.4 |
| 33 | 4 | 30 | 5 | 10 | 10 | 5.25 | 84 | 90.2 |
| 34 | 4 | 32 | 5 | 10 | 12 | 5.50 | 95 | 90.9 |

The 7 mM NTP concentration and Cap concentration of 5.6 mM were selected to pursue the maximum target yield of 9.2 mg/mL. 32 mM MgOAC2 was selected as it worked comparable for different NTP concentrations. Mg+2 is 4 mM above total NTP where RNA Integrity and cap incorporation is maintained above 90% for most of the IVT conditions.

### Example 25

### DTT, Spermidine, and Pyrophosphatase Concentrations

DTT, a reducing agent, can prevent enzyme oxidation in the reaction. DTT concentrations of 5, 10 and 20 mM were studied to determine the optimum concentration. Spermidine, a polyamine, can help T7 RNA polymerase to dissociate from DNA template and swiftly move on to another DNA template for RNA synthesis thereby, increasing transcription efficiency. To check the effect of spermidine on RNA attributes, 0 mM and 2 mM concentrations were selected. Pyrophosphatase concentrations of 0.01 mU/uL and 0.02 mU/uL were studied, as shown in Table 41. The yield, % capped, and % RNA integrity from the reactions are shown in Table 42. Different RNase inhibitor concentrations in control reactions were also studied, as shown in Table 43.

TABLE 41
IVT Process Parameters (DTT, spermidine, pyrophosphatase)

| IVT Components | Units | Varied Concentration studied |
|---|---|---|
| T7 RNA Polymerase, AOF | U/uL | 8, 10 |
| DTT | mM | 5, 10, 20 |
| Spermidine | mM | 0, 2 |
| Pyrophosphatase, AOF | mU/uL | 0.01, 0.02 |

TABLE 42
IVT Process Results

| | Pyrophos- | | | T7 RNA | | | |
|---|---|---|---|---|---|---|---|
| Sample | phatase | DTT | Spermidine | polymerase | Yield | Cap | RNA |
| ID | mU/uL | mM | mM | U/uL | (mg/mL) | % | Integrity % |
| 35 | 0.01 | 5 | 0 | 10 | 5.85 | 99 | 94.3 |
| 36 | 0.01 | 5 | 2 | 10 | 7.3 | 97 | 94.4 |
| 37 | 0.01 | 10 | 0 | 10 | 4.64 | 100 | 94.4 |
| 38 | 0.01 | 10 | 2 | 10 | 6.35 | 98 | 94.2 |
| 39 | 0.01 | 20 | 0 | 10 | 4.91 | 99 | 94.4 |
| 40 | 0.01 | 20 | 2 | 10 | 5.6 | 98 | 94.1 |
| 41 | 0.02 | 5 | 0 | 10 | 6.09 | 98 | 94.5 |
| 42 | 0.02 | 5 | 2 | 10 | 6.75 | 96 | 94.5 |
| 43 | 0.02 | 10 | 0 | 10 | 6.97 | 98 | 93.6 |
| 44 | 0.02 | 10 | 2 | 10 | 6.34 | 98 | 94.4 |
| 45 | 0.02 | 20 | 0 | 10 | 6.88 | 100 | 92.9 |
| 46 | 0.02 | 20 | 2 | 10 | 6.65 | 98 | 93.8 |
| 47 | 0.01 | 5 | 0 | 8 | 5.29 | 100 | 95.5 |
| 48 | 0.01 | 5 | 2 | 8 | 5.78 | 98 | 94.1 |
| 49 | 0.01 | 10 | 0 | 8 | 4.41 | 100 | 94.7 |
| 50 | 0.01 | 10 | 2 | 8 | 5.23 | 98 | 94.9 |
| 51 | 0.01 | 20 | 0 | 8 | 4.85 | 100 | 94.7 |
| 52 | 0.01 | 20 | 2 | 8 | 4.93 | 99 | 94.8 |
| 53 | 0.02 | 5 | 0 | 8 | 5.6 | 100 | 93.5 |
| 54 | 0.02 | 5 | 2 | 8 | 6.61 | 99 | 93.3 |
| 55 | 0.02 | 10 | 0 | 8 | 7.14 | 100 | 94.4 |
| 56 | 0.02 | 10 | 2 | 8 | 6.49 | 98 | 94.3 |
| 57 | 0.02 | 20 | 0 | 8 | 7.11 | 98 | 94.6 |
| 58 | 0.02 | 20 | 2 | 8 | 6.05 | 98 | 94.5 |

TABLE 43
IVT Process Parameters (RNase inhibitor concentrations)

| | Ribolock | | | |
|---|---|---|---|---|
| Sample ID | mU/uL | Yield (mg/mL) | Cap % | RNA Integrity % |
| 59 | 1 | 5.75 | 96 | 87.6 |
| 60 | 0.2 | 5.60 | 96 | 88.5 |
| 61 | 0.08 | 5.65 | 96 | 88.2 |
| 62 | 0.04 | 5.65 | 96 | 86.8 |
| 63 | No ribolock | 5.60 | 97 | 66.2 |

80% of the reactions showed a higher yield with 10 U/uL of T7 RNA polymerase (see Table 42), accordingly, 10 U/uL of T7 RNA polymerase was selected. There was not much difference in RNA quality attributes seen with different concentrations of DTT. However, because DTT helps maintain enzyme activity in the reaction, 5 mM of DTT concentration was selected. The presence of spermidine showed an increase to yield at the lower pyrophosphatase concentration of 0.01 mU/uL, although it didn't show the same impact on yield at pyrophosphatase concentration of 0.02 mU/uL. Spermidine concentration of 2 mM was selected. The control IVT reaction containing no Ribolock RNase inhibitor showed lower RNA integrity. The Ribolock concentration was reduced to 0.08 U/uL as it is effective as 1 U/uL at maintaining RNA integrity. Similar results were seen with reduced Triton X-100 levels (see Table 45).

### Example 26

### Cap and Pyrophosphatase Concentrations

Different concentrations of cap and pyrophosphatase were studied to determine the lowest cap concentration for the developed batch IVT method and whether pyrophosphatase increases RNA yield while maintaining high cap incorporation and RNA Integrity. The cap analogue concentrations studied (mM) were 1, 2, 2.5, 3, 3.5, 4, 4.5, and 5.6, and the pyrophosphatase concentrations studied (mU/uL) were 0.01, 0.02, 0.05, 0.1, 0.2, and 2. The yield, % capped, and % RNA integrity from the reactions are shown in Table 44.

TABLE 44
IVT Process Results

| | Pyrophos- | CleancapAG | Individual | | | |
|---|---|---|---|---|---|---|
| Sample | phatase | (30Me) | NTP/cap | Yield | Cap | RNA |
| ID | mU/uL | mM | ratio | (mg/mL) | % | Integrity % |
| 64 | 0.01 | 5.6 | 1.25 | 6.77 | 98 | 93 |
| 65 | | 4.5 | 1.55 | 6.72 | 97 | 93.3 |
| 66 | | 4 | 1.75 | 5.76 | 98 | 93.3 |
| 67 | | 3.5 | 2 | 5.73 | 98 | 93.5 |
| 68 | | 3 | 2.33 | 6.54 | 95 | 92.5 |
| 69 | | 2.5 | 2.8 | 6.09 | 88 | 93 |
| 70 | | 2 | 3.5 | 5.51 | 91 | 92.9 |
| 71 | | 1 | 7 | 6.78 | 71 | 92.6 |
| 72 | 0.02 | 5.6 | 1.25 | 7.57 | 97 | 92.9 |
| 73 | | 4.5 | 1.55 | 7.32 | 96 | 92.2 |
| 74 | | 4 | 1.75 | 7.49 | 97 | 93.3 |
| 75 | | 3.5 | 2 | 7.48 | 97 | 92.8 |
| 76 | | 3 | 2.33 | 7.44 | 93 | 92 |
| 77 | | 2.5 | 2.8 | 7.29 | 95 | 93.2 |
| 78 | | 2 | 3.5 | 7.03 | 90 | 91.6 |
| 79 | | 1 | 7 | 7.05 | 85 | 91.7 |
| 80 | 0.05 | 5.6 | 1.25 | 7.61 | 98 | 92.2 |
| 81 | | 4.5 | 1.55 | 7.67 | 97 | 91.3 |
| 82 | | 4 | 1.75 | 7.53 | 97 | 91.5 |
| 83 | | 3.5 | 2 | 7.21 | 95 | 92 |
| 84 | | 3 | 2.33 | 7.45 | 95 | 91.8 |
| 85 | | 2.5 | 2.8 | 7.12 | 92 | 90.8 |
| 86 | | 2 | 3.5 | 6.74 | 88 | 91.4 |
| 87 | | 1 | 7 | 6.91 | 72 | 91.9 |
| 88 | 0.1 | 5.6 | 1.25 | 7.75 | 98 | 90.6 |
| 89 | | 4.5 | 1.55 | 7.29 | 97 | 89.2 |
| 90 | | 4 | 1.75 | 7.81 | 97 | 88.1 |
| 91 | | 3.5 | 2 | 7.30 | 94 | 89.3 |
| 92 | | 3 | 2.33 | 7.89 | 97 | 88.6 |
| 93 | | 2.5 | 2.8 | 7.51 | 97 | 88.7 |
| 94 | | 2 | 3.5 | 7.23 | 89 | 87.7 |
| 95 | | 1 | 7 | 7.20 | 73 | 86.8 |
| 96 | 0.2 | 5.6 | 1.25 | 7.44 | 97 | 87.1 |
| 97 | | 4.5 | 1.55 | 7.22 | 97 | 85.7 |
| 98 | | 4 | 1.75 | 7.78 | 97 | 86.5 |
| 99 | | 3.5 | 2 | 7.60 | 96 | 86.8 |
| 100 | | 3 | 2.33 | 7.62 | 91 | 86.7 |
| 101 | | 2.5 | 2.8 | 7.58 | 84 | 85.7 |
| 102 | | 2 | 3.5 | 7.43 | 76 | 84.9 |
| 103 | | 1 | 7 | 7.49 | 63 | 84.3 |
| 104 | 2 | 5.6 | 1.25 | 7.21 | 93 | 80.3 |
| 105 | | 4.5 | 1.55 | 8.45 | 95 | 82.8 |
| 106 | | 4 | 1.75 | 8.00 | 86 | 81.5 |
| 107 | | 3.5 | 2 | 7.16 | 77 | 79.2 |
| 108 | | 3 | 2.33 | 7.65 | 71 | 80.7 |
| 109 | | 2.5 | 2.8 | 7.33 | 77 | 81 |
| 110 | | 2 | 3.5 | 7.50 | 58 | 80.6 |
| 111 | | 1 | 7 | 7.18 | 46 | 80 |

RNA integrity decreased with increase to the pyrophosphatase. RNA yield increased from 0.01 mU/uL of pyrophosphatase to 0.02 mU/uL. However, yield was consistent with increasing pyrophosphatase from 0.02 mU/uL to 2 mU/uL which suggests the reaction condition is balanced in terms of magnesium and pyrophosphatase. Capping efficiency decreased by lowering cap concentration. Cap concentration of 3 mM works equally well across different pyrophosphatase concentration up to 0.1 mU/uL and still maintain >90% cap. The drop in yield with lowering of the cap was observed for most of the conditions.

### Example 27

### Developed Batch Mode IVT Method for RNA Molecules

Teachings from the previous examples were used to develop a batch mode IVT reaction containing: Tris HCl buffer (40 mM concentration and pH of 8), Ribolock (0.08 U/uL), cap analogue (3 mM or 4 mM), ATP (7 mM), CTP (7 mM), GTP (7 mM), P-UTP/UTP (7 mM), Mg(OAc) 2 (32 mM), DTT (5 mM), spermidine (2 mM), DNA template (0.1 ug/uL), pyrophosphatase, AOF (0.02 mU/uL), T7 RNA polymerase, AOF (10 U/uL), with static reaction agitation and run at 37° C. for 120 minutes. Results from control reaction using decreased levels of Triton X-100 (100 times lower than the results shown in Table 43) are shown in Table 45.

TABLE 45
IVT Process Results

| | Ribolock | | | |
|---|---|---|---|---|
| Sample ID | mU/uL | Yield (mg/mL) | Cap % | RNA Integrity % |
| 59c | 1 | 5.52 | 95 | 88 |
| 60c | 0.2 | 5.54 | 96 | 87 |
| 61c | 0.08 | 5.26 | 95 | 87 |
| 62c | 0.04 | 5.44 | 97 | 88.4 |
| 63c | No ribolock | 5.47 | 94 | 58.2 |

### Example 28

### Template Concentration

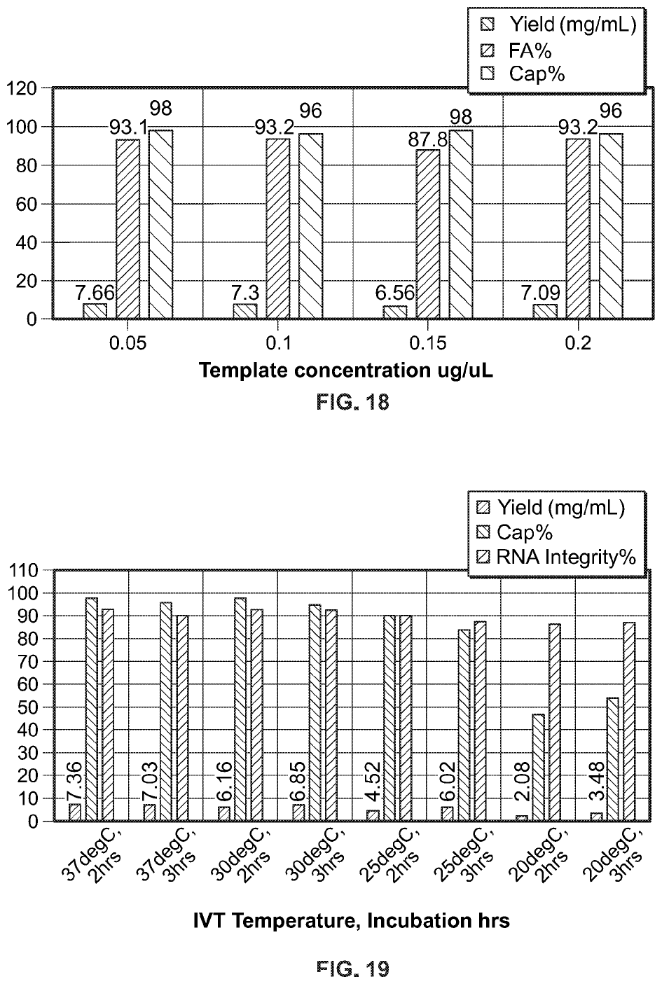

To investigate optimal template concentration, a range of template concentrations were studied. The optimal template concentration can help reduce downstream DNA impurities. The developed IVT method has 0.1 ug/uL of DNA template concentration. Results from these experiments are shown in FIG. 18 and suggest that increasing template concentration does not result in a major yield difference. Accordingly, template concentration can be lowered to 0.05 ug/uL.

### Example 29

### Incubation Temperature and Time

To investigate whether incubation temperature may have any impact on the synthesis of intact RNA, varied temperature and incubation time of IVT was studied. As shown in FIG. 19, as the incubation temperature of IVT lowers to 25° C., a drop in RNA yield is observed. RNA Integrity of ˜90% is maintained at 30° C. for incubation times of both 2 and 3 hrs. However, at 20° C. temperature, cap incorporation is the lowest (about 50%).

### Example 30

### Implementing the Batch Mode Ivt Process on Long RNA Molecules

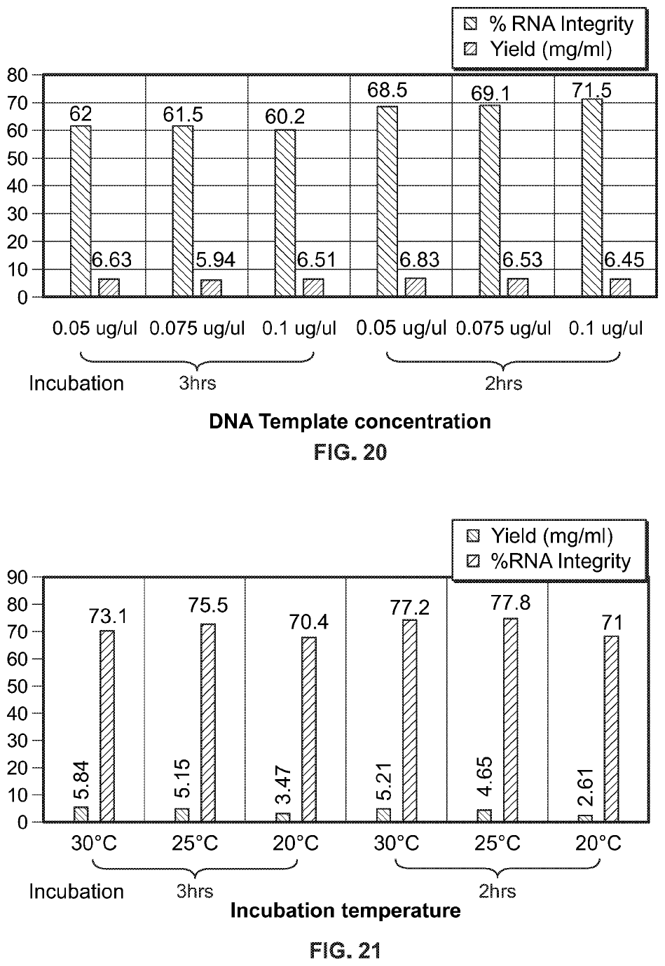

The Batch Mode IVT method described above was tested on a long RNA molecule (˜9.48 KB Self-Amplifying RNA) at 37° C. for incubation times of 2 or 3 hours, and with template concentration of 0.05, 0.075, or 0.1 ug/uL. In addition to the parameters tested, there was no cap molecule added to the reaction. As shown in FIG. 20, at 37° C., the RNA integrity for large RNA molecules (˜9.48 KB) is about 60 to 70% integrity and DNA concentrations of 0.05 ug/uL, 0.075 ug/uL, and 0.1 ug/uL had consistent results for yield and RNA integrity. Therefore, a lower DNA concentration of 0.05 ug/uL was selected. In addition, a longer incubation time resulted in decreased RNA integrity. As a result, a lower incubation temperature was tested.

### Example 31

### Testing Lower IVT Incubation Temperatures and Time

Lower incubation temperatures were investigated to determine whether they can generate higher amounts of intact RNA molecules. Temperatures of 20° C., 25° C., and 30° C. were tested with template concentration at 0.05 ug/uL at incubation times of 2 or 3 hours. As shown in FIG. 21, lowering the incubation temperature for large RNA molecules resulted in a higher RNA integrity compared to 37° C. (FIG. 20). At 25° C. and 30° C., the RNA integrity was ≤77%. 25° C. was selected for the incubation temperature. Also, an increase in the incubation time to 3 hours resulted in a slightly higher RNA yield as compared to a 2 hour incubation time.

### Example 32

### Testing Mg2+ Concentration to Increase RNA Integrity

To achieve the target of ≥80% RNA integrity, a magnesium titration was done to balance the reaction with the concentration of pyrophosphatase and NTPs for long RNA molecules. In addition, two incubation times were tested to obtain optimal yield and RNA integrity. As shown in Table 46, as the magnesium concentration was lowered to 26 mM, RNA integrity increased by 2-4%. 28 mM concentration of Mg2+ was the selected concentration for long RNA molecules because ≥80% of RNA integrity was achieved, and RNA yield was still optimal. In addition, a 3-hour incubation was selected to ensure that ≥5 mg/ml yield is obtained.

TABLE 46
Mg2+ Titration Results

| | Mg2+ | | | | |
|---|---|---|---|---|---|
| Sample | Concentration | Yield | % RNA | Incubation | Temper- |
| ID | (mM) | (mg/mL) | Integrity | (Hr) | ature |
| SA13 | 24 | 4.9 | 79.8 | 2 | 25° C. |
| SA14 | 26 | 4.74 | 81.9 | | |
| SA15 | 28 | 4.92 | 81.4 | | |
| SA16 | 30 | 4.82 | 80.6 | | |
| SA17 | 32 | 4.88 | 80 | | |
| SA18 | 24 | 5.2 | 81.3 | 3 | |
| SA19 | 26 | 5.07 | 81.4 | | |
| SA20 | 28 | 5.2 | 81.1 | | |
| SA21 | 30 | 5.2 | 78.3 | | |
| SA22 | 32 | 5.32 | 77.1 | | |

### Example 33

### Pyrophosphatase Titration and Yield

A pyrophosphatase titration study was done to test whether RNA yield can be increased while maintaining RNA integrity. Pyrophosphatase concentration was tested at 0.02, 0.05, 0.08, 0.1, 0.15, 0.2, and 2 mM with a 3 hour incubation time at 25° C. and a Mg2+ concentration of either 26 or 28 mM. As shown in Table 47, increasing the pyrophosphatase concentration in the reaction resulted in an increase in RNA yield by ˜1-2 mgs at certain concentrations. However, increasing the concentration resulted in RNA integrity to lower; therefore, the selected pyrophosphatase concentration was 0.02 mU/uL.

TABLE 47
Pyrophosphatase Titration Results

| | | Pyrophosphatase | Mg 2+ | |
|---|---|---|---|---|
| Sample | Yield | Concentration | Concentration | % RNA |
| ID | (mg/mL) | (mU/uL) | (mM) | Integrity |
| SA32 | 4.7 | 0.02 | 26 | 79.1 |
| SA33 | 5.76 | 0.05 | | 74.2 |
| SA34 | 6.78 | 0.08 | | 76.3 |
| SA35 | 7.19 | 0.1 | | 75.4 |
| SA36 | 5.56 | 0.15 | | 68.9 |
| SA37 | 6.04 | 0.2 | | 71.3 |
| SA38 | 5.98 | 2 | | 57.7 |
| SA39 | 4.84 | 0.02 | 28 | 81.2 |
| SA40 | 5.19 | 0.05 | | 72.2 |
| SA41 | 5.84 | 0.08 | | 68.9 |
| SA42 | 6.27 | 0.1 | | 71.6 |
| SA43 | 6.18 | 0.15 | | 69.3 |
| SA44 | 6.94 | 0.2 | | 68.1 |
| SA45 | 5.76 | 2 | | 48.3 |

### Example 34

### Batch Mode IVT Method for Long RNA Molecules

Teachings from the previous examples were used to develop a batch mode IVT reaction for long RNA molecules containing: Tris HCl buffer (40 mM concentration and pH of 8), Ribolock (0.08 U/uL), ATP (7 mM), CTP (7 mM), GTP (7 mM), UTP (7 mM), Mg(OAc) 2 (28 mM), DTT (5 mM), spermidine (2 mM), DNA template (0.05 ug/uL), pyrophosphatase, AOF (0.02 mU/uL), T7 RNA polymerase, AOF (10 U/uL), and run at 25° C. for 180 minutes.

### Example 35

### Fed IVT Development—Increasing NTP Concentration to Increase RNA Yield

In addition to lowering cap concentration, a goal for the Fed IVT Method was to increase RNA yield with around 1 mM cap. To increase yield, the NTP concentration was increased in the Batch Mode IVT. As shown in Table 48, increasing the NTP concentration resulted in a decrease in capping efficiency in the Batch Mode IVT while still maintaining RNA integrity. As the target yield is ˜12.5 mg/mL, 9.5 mM of each NTP concentration was selected for the Fed Batch IVT. The following examples explored Mg2+, T7, and pyrophosphatase titration to increase RNA yield and balance the reaction.

TABLE 48
Increased NTP Concentration Results

| | | | CleancapAG | | |
|---|---|---|---|---|---|
| SAMPLE | Yield | NTP | (3OMe) | % RNA | Cap |
| ID | (mg/mL) | (mM) | (mM) | Integrity | % |
| FM07 | 7.01 | 7 | 1 | 93.1 | 88 |
| FM08 | 7.12 | 7.5 | | 93.7 | 87 |
| FM09 | 7.32 | 8 | | 92.6 | 88 |
| FM10 | 6.46 | 8.5 | | 92.7 | 83 |
| FM11 | 6.66 | 9 | | 92.5 | 82 |
| FM12 | 5.21 | 9.5 | | 92.1 | 81 |
| FM13 | 4.78 | 10 | | 91.5 | 71 |
| FM14 | 7.83 | 7 | 4 | 93.5 | 98 |
| FM15 | 8.15 | 7.5 | | 93.6 | 97 |
| FM16 | 8.61 | 8 | | 93.5 | 97 |
| FM17 | 7.81 | 8.5 | | 93.7 | 98 |
| FM18 | 7.88 | 9 | | 93.5 | 98 |
| FM19 | 6.55 | 9.5 | | 92.8 | 97 |
| FM20 | 5.78 | 10 | | 91.6 | 93 |

### Example 36

### Effect of Specific NTPS on Capping Efficiency

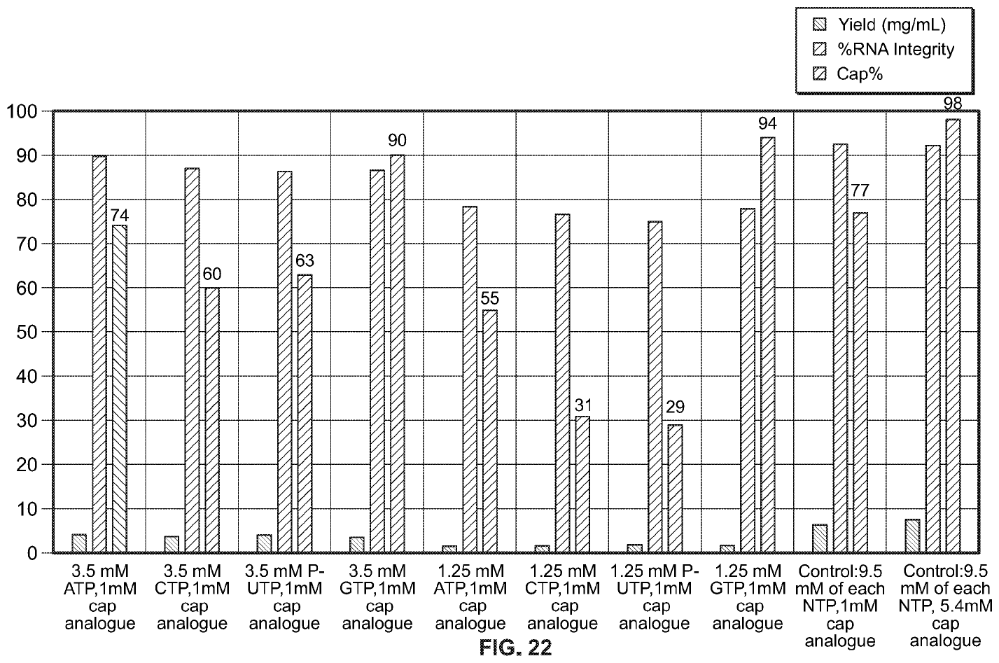

To determine if any of the four NTPs have an impact on cap incorporation, two different ratios of NTP to cap were tested. The two NTP to cap ratios that were tested were 1.25 and 3.5. The NTP concentration was kept at 9.5 mM for all other NTPs that were not being tested in that sample and the cap concentration was 1 mM. Cap analogue refers to CleancapAG (30Me). As shown in FIG. 22, lowering the GTP concentration resulted in higher cap incorporation compared to lowering the concentrations of all other NTPs. For both GTP to cap ratios tested, the cap incorporation was >90%. For all other NTPs, the cap incorporation lowered with the NTP concentration. This shows that the initial concentration of GTP influences the cap percentage. As a result, GTP fed was selected to go forward.

### Example 37

### Increasing RNA Yield

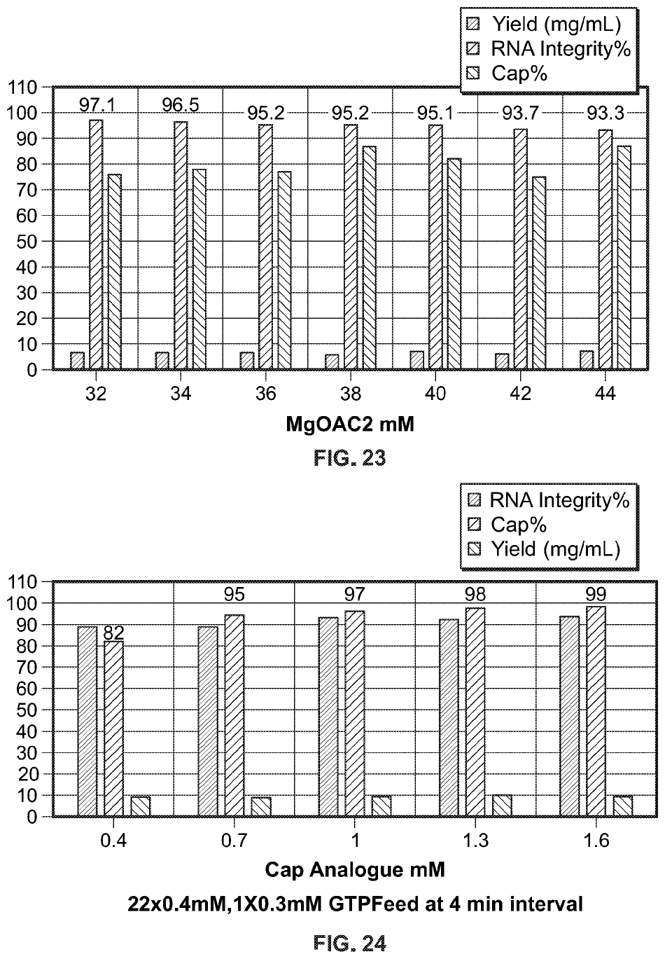

Before studying feeding strategies, IVT was further optimized to increase the yield with the baseline 1 mM cap concentration. To increase RNA yield, a range of Magnesium acetate concentrations were studied, followed by testing of various pyrophosphatase and T7 RNA polymerase concentrations. In this example, the concentration of NTP was 9.5 mM and the concentration of cap was at 1 mM. As shown in FIG. 23, as the concentration of magnesium increased there was slight drop in RNA integrity. RNA yield ranged from 6-7.5 mg/mL. 36 mM of magnesium was selected as a balanced concentration in the reaction and to avoid any negative impact on RNA integrity.

To further increase the yield, various T7 RNA Polymerase and pyrophosphatase concentrations were tested. As shown in Table 49, as pyrophosphatase increased, RNA yield increased for most of the conditions. However, at 0.1 mU/uL pyrophosphatase with 13 U/uL and 15 U/uL of T7 RNA polymerase there is a slight decrease in yield. As a result, 0.08 mU/uL pyrophosphatase was selected. With 15 U/uL and 13 U/uL of T7 RNA polymerase and 0.08 mU/uL of pyrophosphatase, ≤10 mg/mL yield was achieved. However, 15 U/uL was selected to make sure maximum possible yield was achieved.

TABLE 49
Varied Concentrations of T7 RNA
Polymerase and Pyrophosphatase

| | | | | T7 RNA | |
|---|---|---|---|---|---|
| Sample | Yield | % RNA | Cap | polymerase | Pyrophosphatase |
| ID | mg/mL | Integrity | % | (U/uL) | (mU/uL) |
| FM40A | 5 | 93.9 | 66 | 10 | 0.02 |
| FM40B | 7.6 | 95.3 | 84 | 13 | 0.02 |
| FM40C | 7.2 | 94.7 | 84 | 15 | 0.02 |
| FM41A | 6.7 | 93.5 | 77 | 10 | 0.04 |
| FM41B | 6.3 | 94 | 83 | 13 | 0.04 |
| FM41C | 8.7 | 93.4 | 85 | 15 | 0.04 |
| FM42A | 6 | 95.1 | 77 | 10 | 0.06 |
| FM42B | 8.1 | 95.7 | 83 | 13 | 0.06 |
| FM42C | 9.2 | 95.3 | 84 | 15 | 0.06 |
| FM43A | 8.5 | 94 | 78 | 10 | 0.08 |
| FM43B | 9.5 | 94.4 | 79 | 13 | 0.08 |
| FM43C | 10 | 95 | 84 | 15 | 0.08 |
| FM44A | 8.65 | 92.9 | 78 | 10 | 0.1 |
| FM44B | 8.3 | 92.7 | 81 | 13 | 0.1 |
| FM44C | 8.65 | 92.5 | 75 | 15 | 0.1 |

### Example 38

### Determining Amount of GTP Used During Time Interval

To determine the amount of GTP consumed at different time intervals in the developed IVT process, reactions were carried out with the following GTP concentrations: 0.4 mM, 0.8 mM, and 1 mM. The reaction was stopped at various time points and NTP residual was tested. In the first reaction, 0.4 mM of GTP was added to the developed IVT process. The reaction was stopped either at 4, 5, 8, 10, 12, or 15 minutes. At 4 minutes into the reaction, there was still residual GTP present. Accordingly, a 4-minute GTP feed time interval with a feeding concentration of 0.4 mM GTP was selected to test in fed IVT studies.

In the second reaction, 0.8 mM of GTP was added to the developed IVT process. The reactions were stopped either at 5, 8, 10, 12, or 15 minutes and tested for residual GTP. At 8 minutes into the reaction, there was still residual GTP present. Accordingly, a 5-minute and an 8-minute GTP feed time interval with a feeding concentration of 0.8 mM GTP was selected to test in fed IVT studies.

In the third reaction, 1 mM of GTP was added to the developed IVT process. The reactions were stopped either at 5, 8, 10, 12, or 15 minutes and tested for residual GTP. At 8 minutes into the reaction, there was still residual GTP present. Accordingly, a 5-minute and an 8-minute GTP feed time interval with a feeding concentration of 1 mM GTP was selected to test in fed IVT studies.

### Example 39

### Determining Amount of GTP:Cap Analogue

In the following example, different GTP to Cap ratios were tested to determine how low the concentration of cap analogue can be in the reaction with the amount of GTP to feed. The baseline GTP/cap ratio was 0.25 as preferred for ARCA Cap analogue G. In this example, three different GTP concentrations were tested, and the cap concentrations ranged from 0.1 mM to 4 mM. As shown in Table 50, the lowest cap concentration that showed ≥90% cap % in the reaction is 0.4 mM. 0.4 mM cap and any concentration above 0.4 mM allowed for ≥90% cap. In addition, 0.4 mM, 0.8 mM, and 1 mM GTP concentration allows ˜90% cap incorporation. As a result, various GTP:Cap ratios and different GTP feed concentrations were tested in the following studies.

TABLE 50
Varied Ratios of GTP/Cap

| | Clean- | | | | | Yield mg |
|---|---|---|---|---|---|---|
| Sample | capAG | GTP | GTP/Cap | % RNA | Cap | RNA/mL |
| ID | (30Me) mM | mM | Ratio | Integrity | % | rxn |
| FM56 | 0.1 | 0.4 | 4 | 73 | 72 | 0.3 |
| FM57 | 0.4 | | 1 | 59.6 | 95 | 0.58 |
| FM58 | 0.7 | | 0.57 | 71 | 96 | 0.63 |
| FM59 | 1 | | 0.4 | 64.7 | 94 | 0.4 |
| FM60 | 1.3 | | 0.307 | 64.3 | 94 | 0.42 |
| FM61 | 1.6 | | 0.25 | 70.7 | 93 | 0.35 |
| FM62 | 0.4 | 0.8 | 2 | 71 | 93 | 0.59 |
| IFM63 | 0.7 | | 1.14 | 71.4 | 95 | 0.97 |
| FM64 | 1 | | 0.8 | 73.8 | 97 | 0.78 |
| FM65 | 1.3 | | 0.61 | 79.1 | 98 | 0.83 |
| FM66 | 1.6 | | 0.5 | 75.6 | 97 | 0.99 |
| FM67 | 3.2 | | 0.25 | 75.1 | 94 | 0.75 |
| FM68 | 0.4 | 1 | 2.5 | 78.3 | 93 | 0.81 |
| FM69 | 0.7 | | 1.428 | 76.8 | 94 | 1.23 |
| FM70 | 1 | | 1 | 79.5 | 96 | 1.11 |
| FM71 | 1.3 | | 0.769 | 78.5 | 96 | 0.95 |
| FM72 | 1.6 | | 0.625 | 75.1 | 98 | 1.2 |
| FM73 | 4 | | 0.25 | 79 | 97 | 1.26 |
| Control | 1 | 9.5 | 9.5 | 94.8 | 75 | 9.72 |

### Example 40

### Various GTP Feed Concentrations

In the following example, different GTP feed concentrations were tested. In the first study, 0.4 mM of GTP was fed every 4 minutes. In total, there was 9.5 mM GTP added during the IVT process. Different cap analogue concentrations were tested to determine the optimal condition. As shown in FIG. 24, increasing the cap analogue resulted in a higher cap incorporation. ≥0.7 mM cap concentration with 0.4 mM GTP feed was the baseline condition to get >90% cap, 90% RNA Integrity and ˜9 mg/mL yield.

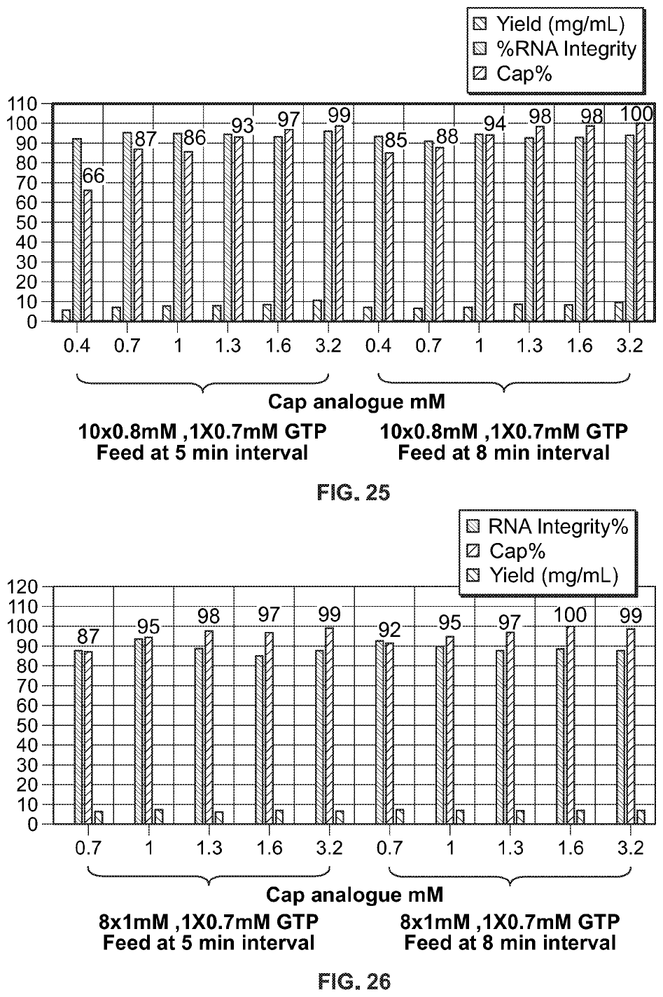

In the second study, 0.8 mM of GTP was fed every 5 or 8 minutes. Various cap analogue concentrations were also tested. As shown in FIG. 25, 0.8 mM GTP fed every 8 minutes resulted in a higher yield and increased cap incorporation compared to 5-minute feeds. In this study, 0.8 mM GTP feed at every 8 minutes with ≥1 mM cap was a baseline condition to get ˜90% RNA integrity, ˜90% cap incorporation with yield of ˜7.5-10 mg/mL.

In the third study, 1 mM of GTP was fed every 5 or 8 minutes. Once again, different cap analogue concentrations were tested. As shown in FIG. 26, once again, 8-minute feeds resulted in a little higher yield compared to 5-minute feeds. From these results, the baseline optimum condition is 0.7 mM cap analogue with 1 mM GTP feeds at every 8 minutes to get >7 mg/mL yield while still maintaining RNA Integrity at ˜90% and cap incorporation at ˜90%.

### Example 41

### RCA Reactions with Varied RCA Template Concentrations

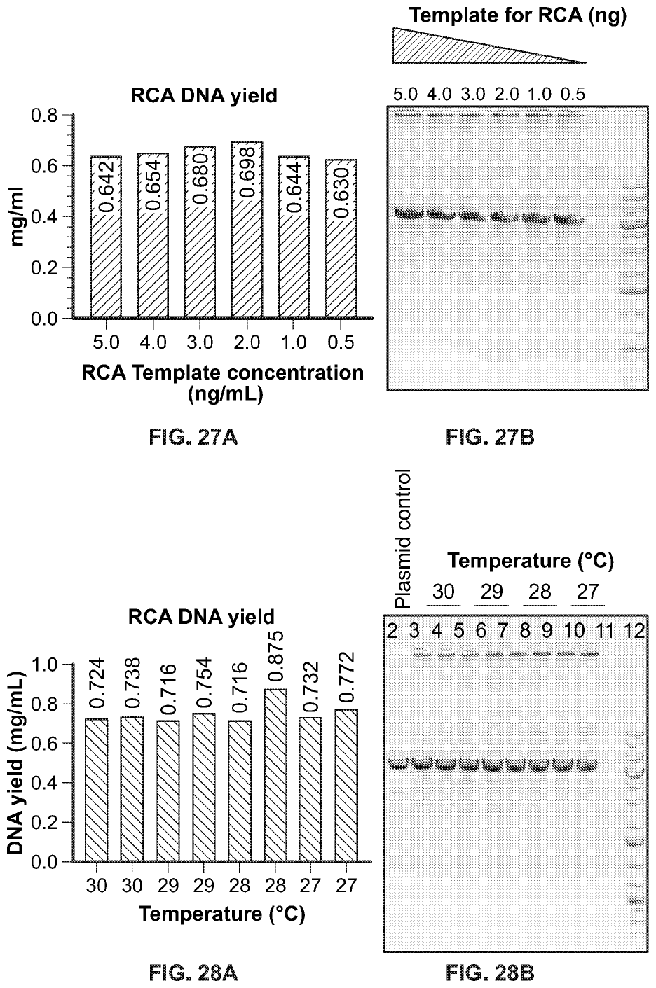

In the following example, as little as one-half to five (0.5-5) nanograms of circular, supercoiled double-stranded DNA (dsDNA) template (containing a transcribable cassette) was used in a rolling circle amplification (RCA) reaction (as described in Table 51) to generate 10⁵-10⁶-fold amplification of the template, yielding a dsDNA concatemer, as shown in FIG. 27A and after linearization in FIG. 27B.

TABLE 51
RCA Reaction with phi29 DNA Polymerase

| | Final | |
|---|---|---|
| Reagents | concentration | |
| Reaction buffer containing: | 1 | X |
| Tris-HCl, (NH₄)₂ SO₄, MgCl₂, KCl, DTT | | |
| Inorganic yeast Pyrophosphatase | 1 | U/mL |
| dATP | 0.5-1.00 | mM |
| dCTP | 0.5-1.00 | mM |
| dTTP | 0.5-1.00 | mM |
| dGTP | 0.5-1.00 | mM |
| 3′ exonuclease resistant random hexamer | 1-10 | μM |
| Phi29 DNA polymerase | 0.50 | U/μL |
| circular dsDNA template | 0.5-5 | ng/mL |

Nuclease free water to rxn volume

The above mixture was mixed with or without heat-denaturation and then incubated for 25-30 hrs at 30° C. Post incubation, phi29 polymerase can be heat inactivated at 65° C. for 10 min. Preferably, the polymerase is not heat inactivated. The reaction can achieve 0.5-0.85 mg/ml of DNA.

As demonstrated herein, RCA reactions can be conducted at a temperature of 27-30° C. for 25-30 hours (see FIG. 28). Additionally, BSA can be removed, RCA buffer composition adjusted, and different dNTP concentrations and Phi29 polymerases used to successfully generate RCA templates for use in in vitro transcription reactions. Finally, both a heat-denaturation step prior to incubation for RCA and a heat inactivation step post-RCA (but pre-linearization) can be omitted from the RCA reaction process. Removing these two steps allow for commercial manufacturing ease and allow for an isothermal process, while still producing surprising quality mRNA as compared to plasmid DNA, as shown below.

### Example 42

### Digestion of RCA DNA without Purification

In the following example, the RCA DNA generated from circular dsDNA template can be enzymatically digested by one or more restriction endonucleases to generate linearized DNA fragments for use as template for in vitro transcription reactions without purification of the RCA DNA prior to enzymatic digestion. Typical reactions require purification of DNA template, reconstitution of the DNA template in buffer, then enzyme digestion of the DNA template. As described herein, the entire reaction (i.e., amplification and linearization) can be performed in the same reaction vessel without losing material, since there is no need to transfer to different vessels for the different reaction steps.

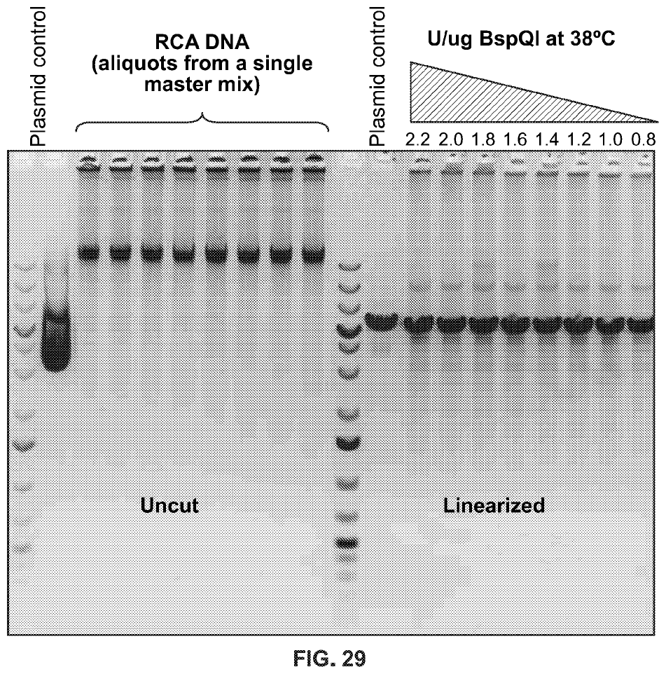

RCA DNA can be digested with a restriction endonuclease to produce linearized dsDNA without the need for DNA purification or additional buffering reagents. 0.8-2.2 U of BspQI (10 U/L, NEB™ #0712) per microgram of RCA DNA was pipetted into the RCA reaction vessel and incubated overnight at 37° C. in a heat block. As shown in FIG. 29, the cut products can be viewed using agarose gel electrophoresis.

### Example 43

### IVT Using Linearized DNA Template without Purification

Linearized RCA DNA or linearized plasmid DNA can be used as a template for in vitro transcription without purification post digestion with restriction endonuclease. Typically, DNA template requires purification and resuspension in buffer prior to its use in an IVT reaction. Surprisingly, this purification step can be eliminated without impact on the yield or quality of the RNA from an IVT reaction.

The linearized RCA DNA prepared as in the above examples will be used as an IVT template to generate mRNA without purification post digestion. Additionally, linearized plasmid DNA without purification can be included as template for IVT. Linearized DNA can also be purified using ethanol/2.5 M NH₄CH₃CO₂ precipitation and re-suspended in nuclease free water. Each DNA sample will be used as the template in an IVT reaction using the parameters shown in Table 52. DNA will be supplemented at 0.1 mg/mL final concentration.

TABLE 52
USTAT v1 IVT Reaction with Linear RCA DNA as Template

| Reagents | Final concentration | |
|---|---|---|
| Tris acetate; pH 8.0 | 50 | mM |
| Mg acetate | 40 | mM |
| NTP mix | 40 | mM |
| Inorganic yeast Pyrophosphatase | 0.003 | U/μL |
| RNase inhibitor | 0.5 | U/μL |
| T7 polymerase | 11 | U/μL |
| Spermidine | 3.5 | mM |
| DTT | 12.5 | mM |
| RCA DNA | 100 | ng/μL |

Nuclease free water to reaction volume

The above reaction will be gently vortexed and incubated at 37° C. for 150 min. DNase I and 1 M CaCl₂) will be added, and the reaction mixture incubated at 37° C. The products will then be purified by LiCl precipitation. Concentration will be measured on nanodrop and RNA integrity will be assessed using fragment analysis. RNA produced using linearized, purified plasmid DNA will be used as a control.

### Example 44

### Unified Sequential Template Amplification and Transcription

Linearized RCA DNA or linearized plasmid DNA can be used as a template for in vitro transcription without purification post digestion with restriction endonuclease. Typically, DNA template requires purification and resuspension in buffer prior to its use in an IVT reaction. Surprisingly, this purification step can be eliminated without impact on the yield or quality of the RNA from an IVT reaction.

An RCA reaction with circular dsDNA (plasmid or synthetic) followed by restriction enzyme linearization and IVT can be performed sequentially, in a single reaction vessel, generating mRNA without any previous purification steps. The process allows for DNA-directed synthesis of RNA molecules of any sequence, ranging in size from short oligonucleotides to several kilobases.

DNA generated from an RCA reaction can be linearized and used as template for IVT reaction in a step-wise reaction in a single vessel without the need for purification or volume transfer, referred to as Unified Sequential Template Amplification and Transcription (USTAT). Template for this single vessel reaction can be as little as 0.5 ng of circular supercoiled dsDNA per mL RCA reaction, without any impact on RNA yield or quality. Heat denaturation of template DNA is not required. The RCA reaction components for such a step-wise single-vessel RCA reaction are as in Table 51.

An RCA reaction was assembled as in Table 51, using 0.5 ng plasmid DNA per mL reaction volume. A similar reaction using commercial buffer conditions was included for comparison. The reaction mixture was gently vortexed and incubated for 25-30 hrs at 30° C. Then, the RCA reactions were digested in the same vessel with a restriction endonuclease to produce linearized dsDNA without the need for DNA purification or additional buffering reagents. 1.0 U of BspQI (10 U/L, NEB, #R0712) per microgram of RCA DNA was pipetted into the RCA reaction vessel and incubated 20-25 hrs at 37° C.

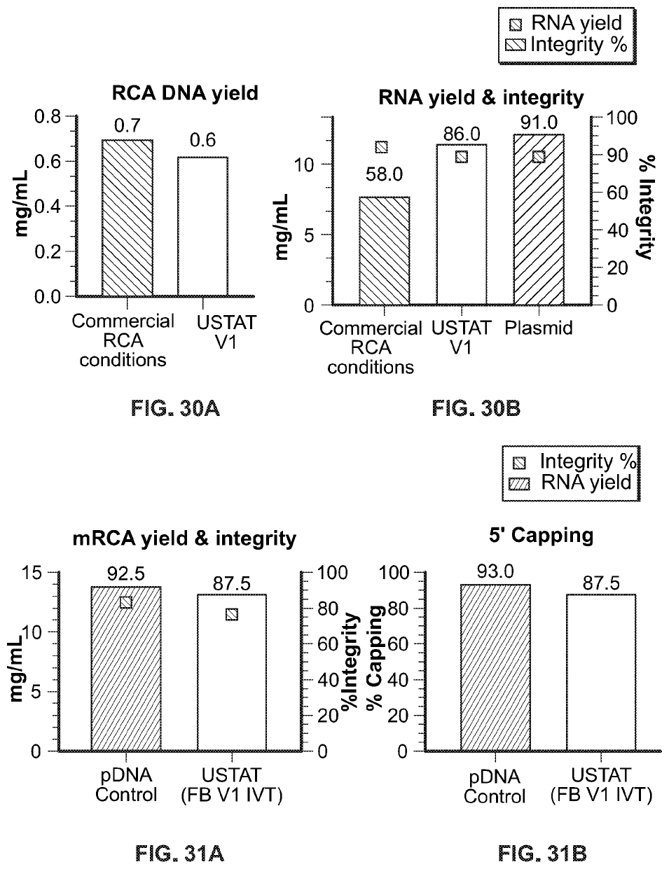

The products of the digestion reaction can be used in a subsequent IVT step without the need for purification. Linearized RCA DNA was used directly in an IVT reaction as in Table 52. Linearized plasmid DNA template was included as a control. The reaction was gently vortexed and incubated at 37° C. for 150 minutes. DNase I and 1 M CaCl₂) were added, and the reaction mixture incubated at 37° C. The products were purified by LiCl precipitation and concentration measured using Nanodrop. RNA integrity was assessed using fragment analysis. Results are shown in FIG. 30.

### Example 45

### USTAT Combined with Improvements to the IVT Reaction

Unified Sequential Template Amplification and Transcription (USTAT) can produce higher quality RNA products when combined with improvements to the IVT reaction.

An RCA reaction was performed, the products digested, and the DNA used as template for IVT without further purification as in Example 44. In a second aspect, plasmid DNA was linearized using 2 U BspQI per microgram DNA and used as template for IVT without purification. A linearized, purified plasmid DNA template for IVT was included as a control.

IVT reactions using the above DNA templates were performed using one of three IVT reaction conditions: a USTAT v1 IVT reaction shown in Table 52, a batch IVT reaction as shown in Table 37 (BV1), or a fed batch IVT reaction as shown in Table 38 (FB V1). Reactions can optionally include components for co-transcriptional capping and/or modified NTPs. RCA DNA or plasmid DNA was supplemented at 0.05-0.1 mg/mL final concentration.

RNA samples were purified by precipitation and concentration measured by UV. The integrity of the resulting RNA was assessed using fragment analysis and an evaluation of the fraction of capped mRNA was performed. Results for RCA DNA template compared to linearized plasmid template (purified control or unpurified) in IVT using USTAT V1, BV1, or FB

V1 IVT processes are shown in Table 53.

TABLE 53
Comparison of RCA DNA Template to Linearized Plasmid

| | IVT | | Yield | 5′Cap | Integrity |
|---|---|---|---|---|---|
| Reaction | Process | DNA | (g/L) | (%) | (%) |
| A | USTAT V1 | Linearized pDNA | 9.0 | 66 | 91.9 |
| B | | Unpurified | 8.7 | 67 | 87.6 |
| | | linearized pDNA | | | |
| C | | USTAT RCA | 6.7 | 49 | 84.4 |
| D | BV1 | Linearized pDNA | 8.5 | 82 | 84.1 |
| E | | Unpurified | 8.7 | 81 | 83.0 |
| | | linearized pDNA | | | |
| F | | USTAT RCA | 9.5 | 70 | 87.4 |
| G | FB V1 | Linearized pDNA | 13.1 | 93 | 92.5 |
| H | | Unpurified | 13.0 | 94 | 92.1 |
| | | linearized pDNA | | | |
| I | | USTAT RCA | 11.2 | 87 | 87.5 |

### Example 46

### Scalability of USTAT Reaction

The volume of a Unified Sequential Template Amplification and Transcription (USTAT) reaction can be increased to enable production of larger amounts of RNA.

The RCA DNA amplification reaction was performed at 100 mL scale and products were linearized using BspQI as in Example 44. The DNA was supplemented at 0.1 mg/ml to an FB V1 IVT reaction shown in Table 38. A reaction using linearized, purified plasmid DNA template was included as control. RNA samples were purified by precipitation and concentration determined by UV. RNA integrity was determined by FA and % capping was evaluated by an LC-UV assay. Results are shown in FIG. 31.

### Example 47

### Next Gen Sequencing of Linearized USTAT RCA DNA

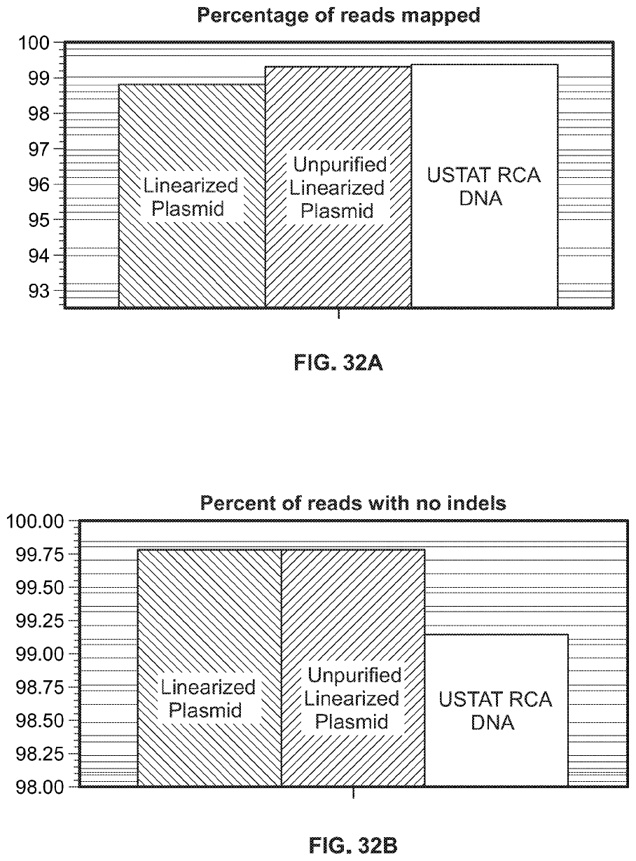

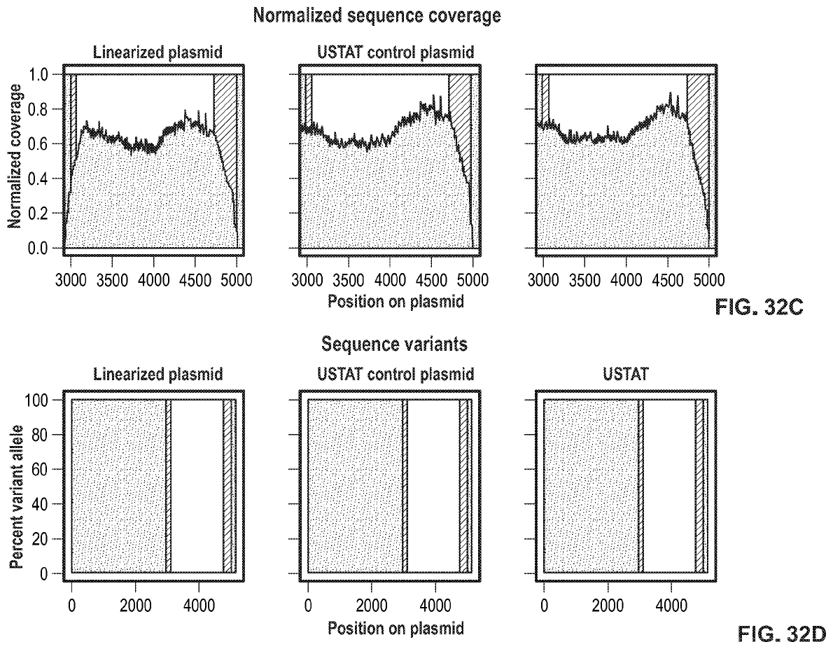

Next generation sequencing can be used to evaluate the sequence fidelity of DNA amplified in the RCA reaction relative to plasmid template. Sequences from linearized and purified plasmid DNA, linearized but unpurified plasmid DNA, and linearized RCA DNA were evaluated for the presence of insertions/deletions or for any sequence variants. No variants were observed within the transcription template, as shown in FIG. 32.

### Example 48

### USTAT RNA Expression in Cell Culture

The RNA produced using USTAT can be transfected into HEK293 cells, resulting in protein expression similar to that observed using RNA derived from a plasmid DNA template.

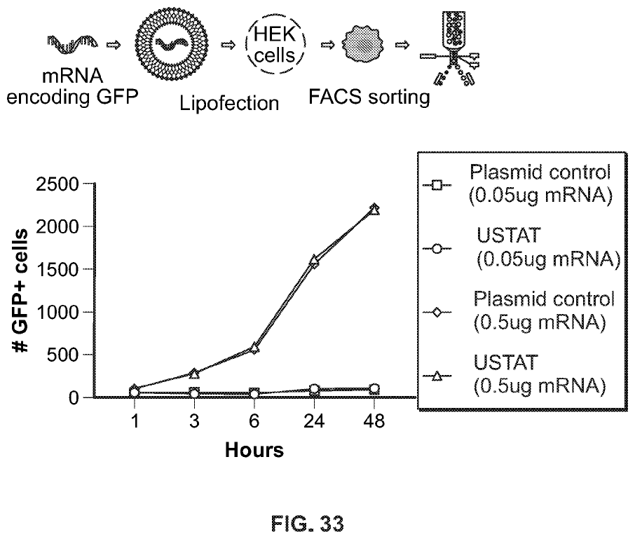

USTAT RCA DNA or linearized plasmid control DNA, each containing a transcription cassette encoding GFP, were used as template in an IVT reaction as in Table 37. Reactions were run in duplicate to produce uncapped as well as co-transcriptionally capped RNA. The RNA was purified using lithium chloride precipitation, resuspended in water and concentration was measured using Nanodrop. A total of 5 μg of RNA was transfected per well, with 0.5 ug or 0.05 ug of capped RNA and the balance uncapped RNA. Transfected cells were allowed to recover for 1 hour before evaluating GFP expression by FACS (BD FACSMELODY™). The number of GPF expressing cells per 5000 counts is shown in FIG. 33. Control transfections using only uncapped RNA did not exhibit measurable GFP expression.

### Example 49

### USTAT Reactions Using Variable Sized Templates

A USTAT reaction can be used to produce RNA of various sizes, from 1.4 kb to over 10 kb. A set of plasmids containing a transcription cassette for various sized RNAs was used as template in USTAT RCA as in Example 44: Unified Sequential Template Amplification and Transcription. The linearized DNA was added to an IVT reaction comprised of components in Table 37. RNA was purified using lithium chloride precipitation and resuspended in water. The yield and integrity for reactions using constructs with variable sized IVT transcript templates are shown in Table 54. RCA DNA yield, RNA yield and RNA integrity are shown. Plasmid DNA template was included as a control. RNA integrity was measured using FA.

TABLE 54
Comparison of Reactions Using Variable
Sized IVT Transcript Templates

| | | RCA DNA | | |
|---|---|---|---|---|
| | Transcript size | yield | RNA Yield | Integrity |
| Construct | (kb) | (mg/mL) | (mg/mL) | (%) |
| pDNA C09 | 1.8 | 0.874 | 4.8 | 85 |
| pDNA 89 | 1.4 | 0.874 | 5.1 | 77 |
| pDNA 92 | 4.3 | 0.978 | 5.6 | 81 |
| pDNA 93 | 5.9 | 0.920 | 5.2 | 73 |
| pDNA 94 | 7.7 | 0.854 | 5.0 | 57 |
| pDNA 96 | 11 | 0.890 | 5.3 | 54 |
| pDNA control | 1.8 | n/a | 5.4 | 91 |

### Example 50

### One Step Template Assembly and USTAT

A circular dsDNA template can be assembled from linear dsDNA fragments for use in a USTAT reaction without any cleanup or purification steps prior to DNA amplification. This allows for template assembly, DNA amplification, linearization and IVT to be performed sequentially in a single vessel, without the need for purification steps. The linear dsDNA fragments can be generated by in vitro DNA synthesis, enzymatic digestion of a fragment from a plasmid or by PCR.

Linear dsDNA fragments containing a sequence for a gene of interest (GOI) were either obtained from a commercial supplier (IDT) or generated by PCR. A second set of fragments that include a promoter sequence, 5′ and 3′ UTRs, and a poly A sequence, were generated either by isolating a fragment from a plasmid using enzymatic digestion or by PCR. As an alternative, a single dsDNA fragment was amplified by PCR from a plasmid, to be circularized in the next step. Linear dsDNA was combined in a reaction with enzymes and buffer (NEBuilder HiFi assembly mix or similar) and the resulting circular dsDNA combined directly with RCA reaction components as in Table 51, using 0.5 ng DNA per mL reaction volume. This reaction was used to generate mRNA as in Example 44.

Linearized RCA DNA was used directly in an IVT reaction as in Table 52. The reaction was gently vortexed and incubated at 37° C. for 150 minutes, followed by purification by LiCl precipitation. RNA concentration was measured using Nanodrop and RNA integrity was assessed using fragment analysis. Results are shown in Table 55.

TABLE 55
Comparison of mini-circle USTAT template to plasmid DNA

| | | | RCA DNA | RNA | |
|---|---|---|---|---|---|
| | Fragment | Fragment | yield | Yield | RNA |
| Template | 1 | 2 | (mg/mL) | (mg/mL) | Integrity |
| m01 | Synthetic | Digest | 0.856 | 8.0 | 76 |
| m02 | PCR | Digest | 0.864 | 7.7 | 74 |
| m03 | PCR | PCR | 0.712 | 7.7 | 75 |
| m04 | Synthetic | PCR | 1.620 | 7.0 | 66 |
| m05 | Single PCR | | 0.674 | 7.3 | 77 |
| pDNA | n/a | n/a | 0.732 | 7.3 | 76 |
| CA09 | | | | | |

### Example 51

### USTAT with Buffer Modifications

The components of the buffer formulation for unified sequential template amplification and transcription (USTAT) can be adjusted to optimize for mRNA product quality. In the following example, modification to the reaction components was assessed along with lower DNA template addition for effects on mRNA integrity and yield.

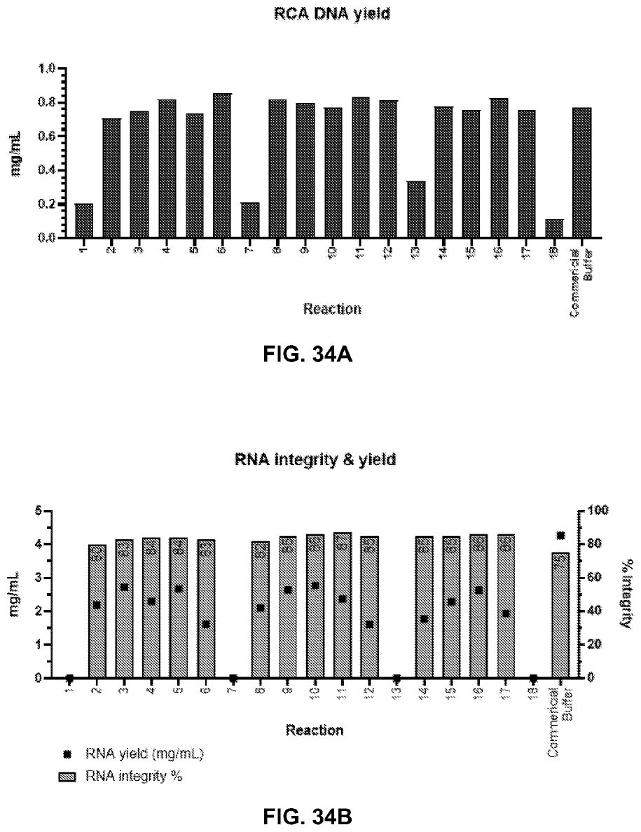

RCA reactions were assembled as in Table 56, using 0.05 ng plasmid DNA per mL reaction volume. A reaction using a commercial buffer composition was included as control. The reaction mixture was gently vortexed and incubated for 22-30 hrs at 30° C. Then, the RCA reactions were digested in the same vessel with a restriction endonuclease to produce linearized dsDNA without the need for DNA purification or additional buffering reagents. 1.0 U of BspQI (NEW ENGLAND BIOLABS) per mL of RCA reaction was pipetted into the RCA reaction vessel and incubated 20-25 hrs at 37° C. RCA DNA yield is shown in FIG. 34A. The linearized DNA was used directly as template for IVT.

TABLE 56
USTAT RCA reaction with buffer composition options

| | Final | |
|---|---|---|
| Reagents | concentration | |
| Reaction buffer containing: | 1 | X |
| Tris buffer, (NH₄)₂ SO₄, MgCl₂, KCl, DTT | | |
| or | | |
| Tris buffer, (NH₄)₂ SO₄, MgCl₂, NaCl, DTT | | |
| Inorganic yeast Pyrophosphatase | 0.5 | U/mL |
| dATP | 0.5-1.00 | mM |
| dCTP | 0.5-1.00 | mM |
| dTTP | 0.5-1.00 | mM |
| dGTP | 0.5-1.00 | mM |
| 3′ exonuclease resistant random primer | 1-10 | μM |
| Phi29 DNA polymerase | 0.50 | U/μL |
| circular dsDNA template | 0.05-5 | ng/mL |

Nuclease free water to reaction volume

The DNA was supplemented at 0.1 mg/mL to a BV1 IVT reaction as shown in Table 37. RNA samples were purified by precipitation and concentration determined by UV. RNA integrity was determined by FA and results are shown in FIG. 34B.

### Example 52

### USTAT with Varied Priming Options and Template Length

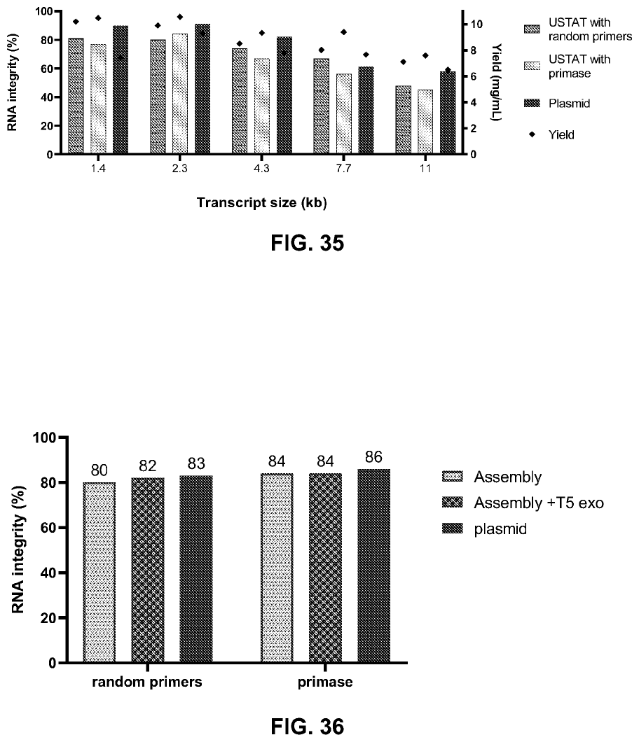

An evaluation of priming options was performed using dsDNA templates for USTAT that produce mRNAs of varying lengths. A set of plasmids containing a transcription cassette for various sized RNAs from 1.4 kb to 11 kb was used as template for USTAT RCA. Reactions were assembled as in Table 56 and included either random primers or 10 μg/mL primase enzyme to generate priming sites for phi29 DNA polymerase. Linearized RCA DNA and the corresponding linearized plasmid template control were used as template for BV1 IVT as in Example 51. RNA samples were purified by precipitation and concentration determined by UV. RNA integrity was determined by FA and results are shown in FIG. 35.

### Example 53

### Improvements to Synthetic Template Assembly for USTAT

Linear dsDNA fragments can be assembled and used directly as template in USTAT reactions for mRNA production, which provides an opportunity for fully synthetic template generation. Several improvements to the synthetic strategy have been incorporated. The design parameters for template sequence were modified to include recoding the gene of interest to eliminate homopolymer sequences greater than 5 bp. This modification will reduce the occurrence of insertions or deletions produced by phi29 at homopolymer sequences. Second, since the assembled dsDNA is used as template for USTAT without exonuclease treatment or purification steps, the assembly design has been adjusted to eliminate sequence downstream of the T7 promoter and reduce potential transcription from any linear fragments that are carried through to the IVT step.

A linear dsDNA fragment containing a gene of interest and 5′ UTR sequence was obtained from a commercial supplier. A second fragment containing a T7 RNA polymerase promoter, 3′ UTR and poly A sequence was digested from plasmid DNA. The fragments were assembled using a reaction mix containing a DNA polymerase, T5 exonuclease and Taq ligase according to a method previously described [Gibson et al., Nat Methods 6 (2009)]. Each assembly reaction was performed with and without a T5 exonuclease digestion step for comparison and the assembled DNA mixed directly with USTAT components as in Table 56, using 0.05 ng DNA per mL reaction. Linearized RCA DNA was used as template for BV1 IVT as in Example 51. RNA samples were purified by precipitation and concentration determined by UV. RNA integrity was determined by FA and results are shown in FIG. 36.

### Example 54

### Post Transcriptional Enzymatic Capping

Post-transcriptional enzymatic capping via the Vaccinia virus Capping Enzyme is applicable for longer RNA molecules as well as all other sizes of RNAs. Uncapped RNA made by the in vitro transcription method described in Example 37 was post-transcriptionally enzymatically capped by Vaccinia virus Capping Enzyme as recommended by the manufacturer (NEW ENGLAND BIOLABS) to generate cap1 RNA. RNA was also pre-treated at different temperatures to understand the effect of pre-treatment temperature on % RNA integrity and % cap. These results are shown in Table 57.

TABLE 57
Effect of Pre-treating RNA at Various Temperatures

| | Pre-Heating RNA | % RNA | |
|---|---|---|---|
| No | Temperature | Integrity | % Cap |
| Uncap 58 | N/A | 81.3 | N/A |
| VCE1 | 65° C., on ice for 5 min (Control) | 69.9 | 100 |
| VCE2 | 37° C. for 5 min | 73.9 | 100 |
| VCE3 | 25° C. for 5 min | 74.4 | 100 |

Table 57 shows that preheating RNA at a lower temperature of 25° C. has less impact on loss of % RNA Integrity. Cap incorporation was maintained at >90% across all of the conditions tested.

To understand whether % RNA integrity is maintained at lower post enzymatic capping reaction temperature, the uncapped RNA was post-transcriptionally enzymatically capped at incubation temperature 25° C. and 30° C. for 30 min and 60 min respectively. These results are shown in Table 58.

TABLE 58
Effect of Time and Pre-treatment of RNA

| | Pre-Heating | | Reaction | | |
|---|---|---|---|---|---|
| | RNA | Reaction | Incubation | % RNA | |
| No | Temperature | Temperature | time | Integrity | % Cap |
| Uncap 58 | N/A | N/A | N/A | 78.7 | N/A |
| VCE4 | 25 C. | 25 C. | 30 mins | 77.6 | 97 |
| VCE5 | | | 60 mins | 77.2 | 100 |
| VCE6 | | 30 C. | 30 mins | 78.4 | 99 |
| VCE7 | | | 60 mins | 67.3 | 100 |

Table 58 shows that lower reaction temperature 25° C. and 30° C. with incubation time 30 min results in minor loss of % RNA Integrity. There is higher loss in % RNA Integrity at incubation temperature 30° C. for 60 min.

### Example 55

### Exploration of Buffering Agents in the IVT Reaction

HEPES can be used as a buffering agent in an IVT reaction to manufacture RNA Kern and Davis, Biotechnol Prog 15:174-84 (1999)]. To assess the impact of HEPES on the BV1 process, a OFAT (one factor at a time) study was performed. Modeled after the ranges explored for Tris described above, this experiment tested concentrations of 20-60 mM HEPES and pH's of 7.5-8.5. No Tris pH 8.0 was added to the reaction and HEPES was used instead, but all other parameters of BV1 as described in Table 37 were kept consistent. As shown in Table 59, at higher pH and higher HEPES concentrations, the IVT yield may decrease slightly. However, all tested conditions were effective for the IVT reaction and generated intact mRNA.

TABLE 59
HEPES and pH One Factor at a Time Analysis

| | Starting | | Yield (g/L | |
|---|---|---|---|---|
| | HEPES Level | Targeted HEPES | starting | Integrity |
| Condition | (mM) | pH | volume) | (%) |
| A | 20 | 7.5 | 8.5 | 93 |
| B | 20 | 8 | 8.4 | 94 |
| C | 20 | 8.5 | 8.5 | 94 |
| D | 40 | 7.5 | 8.5 | 95 |
| E | 40 | 8 | 8.3 | 95 |
| F | 40 | 8.5 | 6.9 | 95 |
| G | 60 | 7.5 | 8.3 | 95 |
| H | 60 | 8 | 7.2 | 95 |
| I | 60 | 8.5 | 5.4 | 94 |

An additional study was performed to determine whether the buffering agent, Tris, could be delivered through other raw materials as opposed to a stock solution. Tris-buffered NTPs were explored as an alternative to sodium-stabilized NTPs. These Tris NTPs had been titrated to a pH of 7.3-7.5 with an unspecified quantity of Tris base (THERMO FISHER). NaOH was added to the experimental conditions to keep starting pH constant. The process BV1 as described in Table 37 was run in a high-throughput manner. Condition A shown in Table 60 used sodium-stabilized NTPs while conditions B-E used the Tris NTPs. The use of Tris NTPs was associated with a slight decrease in FA, but still had decent product quality. Surprisingly, Condition B, which did not have any additional Tris besides what was supplied in the NTPs, performed most comparably to the control. This demonstrates that if the individual IVT raw materials have sufficient buffering capability, additional buffer may not always be needed to stabilize the reaction system.

TABLE 60
Tris NTPs and Tris stock solution

| | Concentration | | | |
|---|---|---|---|---|
| | of Tris from | Tris or | Yield (g/L | |
| | Stock Solution | Sodium | starting | Integrity |
| Condition | (mM) | NTPs? | volume) | (%) |
| A | 40 | Sodium | 8.02 | 95 |
| B | 0 | Tris | 7.55 | 88 |
| C | 10 | Tris | 6.57 | 87 |
| D | 20 | Tris | 7.16 | 88 |
| E | 30 | Tris | 6.73 | 88 |

### Example 56

### IVT Platform Batch Process Applications

The IVT processes described herein are generally applicable to a wide variety of mRNA constructs. The approaches described herein expedite process development and enable faster delivery of life-saving products. To demonstrate the successful application of this approach, process BV1 (described in Table 37) was used to generate unique mRNA molecules. For these reactions, RNase inhibitor was increased from 100 U/mL to 1000 U/mL to reduce the risk of RNase contamination from plasmid DNA preps, but all other parameters matched BV1 (described in Table 37). In a series of small-scale high-throughput IVT reactions, a series of OFAT (one factor at a time) studies were performed. In each of these conditions, either 5′ UTR, coding sequence, or sequence length was changed. As shown in Table 61, the IVT BV1 process is effective in producing mRNA across 13 different construct designs.

TABLE 61
BV1 Process Evaluation on 5′ UTRs,
Coding Sequence, and Coding Sequence Lengths

| | | Yield (g/L |
|---|---|---|
| Construct | Varied | starting |
| Name (kb) | Parameter | volume) |
| RNA11 (0.7) | 5′ UTR #1 | 5.69 |
| RNA12 (0.7) | 5′ UTR #2 | 5.86 |
| RNA13 (0.7) | 5′ UTR #3 | 6.90 |
| RNA14 (0.7) | 5′ UTR #4 | 4.81 |
| RNA15 (0.7) | Coding sequence | 6.03 |
| RNA16 (0.6) | Coding sequence | 7.95 |
| RNA17 (0.9) | Coding sequence | 6.06 |
| RNA18 (1.7) | Coding sequence | 6.62 |
| RNA19 (1.4) | Coding sequence | 7.11 |
| RNA20 (2.0) | Coding sequence | 7.20 |
| RNA21 (3.0) | Coding sequence | 8.33 |
| RNA22 (4.3) | Coding sequence | 5.42 |
| RNA23 (5.9) | Coding sequence | 5.61 |

The process BV1 was further evaluated across a separate set of 7 plasmid DNA constructs. No modifications to the BV1 process were made when executing this evaluation. The data shown in Table 62 further confirms that BV1 can successfully product high quality mRNA across constructs with a wide variety of coding sequences. Furthermore, the mRNA generated across BV1 is of high quality, with high FA integrity values and a 5′ capping percentage of >80%.

TABLE 62
BV1 Process Evaluation on Varying Coding Sequences

| | Yield (g/L | | |
|---|---|---|---|
| Construct | starting | | |
| Name (kb) | volume) | Integrity (%) | 5′Cap (%) |
| RNA1 (2.13) | 8.8 | 94 | 83 |
| RNA5 (2.16) | 8.5 | 91 | 84 |
| RNA6 (1.87) | 9.2 | 92 | 87 |
| RNA7 (2.16) | 8.8 | 79 | 83 |
| RNA8 (1.87) | 9.0 | 94 | 83 |
| RNA9 (2.16) | 8.8 | 92 | 84 |
| RNA10 (1.87) | 9.5 | 87 | 84 |

### Example 57

### NTP Intensification in IVT Fed Batch Reactions

To enhance the IVT processes for generally applicable across multiple constructs (e.g., sizes, UTRs, etc.), the fed-batch process was further refined by intensifying levels of NTPs. The goal of this experiment was to determine the levels of NTPs that can be fed while still manufacturing high quality mRNA (e.g., high integrity and capping efficiency) and yield. NTPs were significantly increased and, as shown in Table 63, NTPs can be increased while maintaining high quality mRNA (i.e., integrity >90% and capping efficiency of >85%) and RNA yield >10 g/L starting volume. Furthermore, while data also showed Mg concentration limitation at high NTPs as indicated by lower yields (e.g., 10.6 v 14.5 g/L, conditions J and K, respectively), some conditions (e.g., K) with increased Mg concentration surprisingly showed decreased capping efficiency. This observation is consistent with data in Table 63 that showed 10 increased initial Mg levels impacted capping efficiency.

TABLE 63
NTP Intensification Impacts Yield, Integrity, and 5′ Cap

| | | | | | | | | | | | Yield | | |
|---|---|---|---|---|---|---|---|---|---|---|---|---|---|
| | ATP | | CTP | | GTP | | pUTP | | Magnesium | | (g/L, | | |
| | (mM) | | (mM) | | (mM) | | (mM) | | (mM) | | starting | Integrity | 5′Cap |
| Condition | Initial | Final | Initial | Final | Initial | Final | Initial | Final | Initial | Final | volume) | (%) | (%) |
| A | 5 | 18 | 5 | 18 | 1 | 15 | 6 | 13 | 36 | 36 | 13.4 | 94 | 89 |
| B | 5 | 18 | 5 | 18 | 1 | 13 | 6 | 13 | 36 | 36 | 14.0 | 93 | 91 |
| C | 5 | 18 | 5 | 18 | 1 | 11 | 6 | 13 | 36 | 36 | 13.5 | 94 | 90 |
| D | 7 | 20 | 7 | 20 | 1 | 15 | 6 | 13 | 36 | 36 | 13.0 | 95 | 91 |
| E | 7 | 20 | 7 | 20 | 1 | 13 | 6 | 13 | 36 | 36 | 13.6 | 95 | 92 |
| F | 7 | 20 | 7 | 20 | 1 | 11 | 6 | 13 | 36 | 36 | 12.5 | 94 | 92 |
| G | 9 | 22 | 5 | 18 | 1 | 15 | 6 | 13 | 36 | 36 | 13.9 | 95 | 92 |
| H | 9 | 22 | 5 | 18 | 1 | 13 | 6 | 13 | 36 | 36 | 14.2 | 95 | 92 |
| I | 9 | 22 | 5 | 18 | 1 | 11 | 6 | 13 | 36 | 36 | 13.1 | 94 | 92 |
| J | 11 | 24 | 9 | 22 | 1 | 16 | 4 | 15 | 36 | 36 | 10.6 | 95 | 91 |
| K | 11 | 24 | 9 | 22 | 1 | 16 | 4 | 15 | 50 | 50 | 14.5 | 94 | 86 |
| L | 9 | 26 | 5 | 22 | 1 | 15 | 6 | 15 | 36 | 36 | 11.1 | 94 | 89 |
| M | 9 | 26 | 5 | 22 | 1 | 15 | 6 | 15 | 50 | 50 | 13.7 | 94 | 89 |

### Example 58

### MG Addition Strategy in IVT Reactions

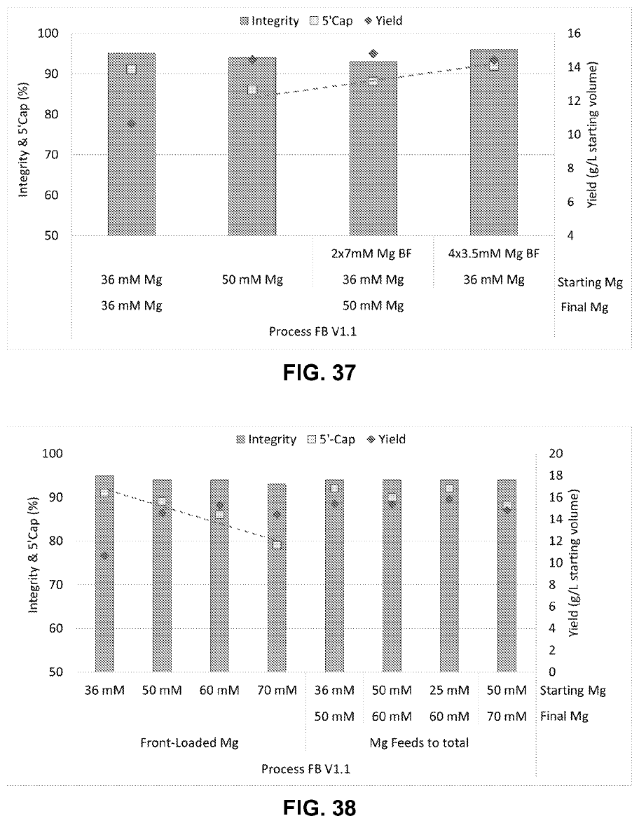

To investigate the impact of Mg concentration in an IVT reaction, Mg addition strategy was evaluated in a fed-batch process with intensified NTP concentrations. In this study, conditions from row J of Table 63 (referred to as ‘Process FBV1.1’) were used as a baseline process for this example. The specific details of this process are listed in Table 65. Here, two Mg addition strategies were evaluated: 1) adding the total volume of Mg in the beginning of the IVT reaction (‘front-loaded’); and 2) starting with a lower concentration of Mg in the beginning of the IVT reaction and feeding the remaining Mg to achieve the total or final concentrations indicated. FIG. 37 shows that increasing Mg levels to 50 mM significantly increased mRNA yield. However, the increased yield unexpectedly resulted in lower capping efficiency when increased Mg levels were front-loaded. Surprisingly, feeding some Mg during an IVT reaction increased capping efficiency. Importantly, increased frequency of Mg additions resulted in further surprising improvement of 5′ capping efficiency.

Next, the impact of total Mg concentration on RNA quality attributes and yield was further investigated. As shown in FIG. 38, increased front-loaded Mg resulted in significantly reduced RNA 5′ capping efficiency with minimal negative impact on RNA integrity and yield. However, starting lower initial Mg concentration and supplementing remaining Mg through feeds markedly improved capping efficiency. Interestingly, starting even lower Mg concentration and feeding the remaining to total resulted in further increase in capping efficiency as shown by conditions with a total of 60 mM Mg concentration but with different starting Mg concentrations. Together, these data demonstrate that the feeding strategy developed in this work remarkably improves RNA quality attributes and yield. As described herein, increased levels of front-loaded Mg concentration negatively impacts RNA quality. However, feeding Mg concentration improved capping efficiency with minimal to no impact on integrity and yield. In these data, 4 Mg bolus feeds were used to bring concentrations to total (e.g., for condition starting with 36 mM Mg, 4 feeds of 3.5 mM each were added at equally spaced time points during IVT bringing total to 50 mM and similarly starting at 25 mM ending at 60 mM resulted in 4 feeds of 8.75 mM each). Dashed lines in FIG. 38 show the overall trend.

### Example 59

### Impact of IVT Incubation Time, Capping Molecule Concentration, and GTP Concentration on IVT Performance

The goal of this experiment was to investigate the impact of capping molecule concentration, final GTP concentration, and increased IVT time on yield, integrity and 5′ Cap in an NTP-intensified fed batch process. In this example, automated microbioreactor systems were used and Process FB V1.2 was used as a baseline process (specific details provided in Table 65). The modifications to each condition from Process FB V1.2 are outlined in Table 64, below. As shown in Table 64, increased yield was observed at an IVT time of 180 minutes compared to the same conditions run at 150 minutes. Additionally, yields were increased to above 16 g/L when targeting a final GTP concentration of 16 mM compared to 13.5 mM. No impact to integrity was observed at 180 minutes compared to 150 minutes. Additionally, no major impact to 5′ cap was observed between 150 and 180 minutes. A dose dependent relationship was observed between % capped RNA and the concentration of capping molecule, with 4 mM 5′ cap analogue producing the highest % capped RNA. Overall, this data demonstrates the success of NTP intensification and the importance of IVT component feeding strategy (e.g., Mg) to enhance RNA quality attributes and yields at a target IVT time of 180 minutes. This process can thus be modified (for example, modifying the total IVT incubation time) to accommodate a range of RNA construct sizes.

TABLE 64
Capping molecule Concentration, Final GTP, and
Total IVT Time Impacts on Yield, and 5′ Cap

| | | | | Total | | | |
|---|---|---|---|---|---|---|---|
| | | 5′Cap | Final | IVT | Yield (g/L | | |
| | | Analogue | GTP | Time | starting | Integrity | 5′Cap |
| Condition | Process | (mM) | (mM) | (min) | volume) | (%) | (%) |
| A | FB V1.2 | 4 | 16 | 150 | 15.7 | 93 | 93 |
| B | | 2.5 | 16 | 150 | 15.0 | 94 | 88 |
| C | | 2 | 16 | 150 | 14.5 | 93 | 86 |
| D | | 4 | 13.5 | 150 | 14.5 | 93 | 94 |
| E | | 2.5 | 13.5 | 150 | 14.3 | 94 | 91 |
| F | | 2 | 13.5 | 150 | 13.6 | 94 | 89 |
| G | | 4 | 16 | 180 | 16.6 | 94 | 94 |
| H | | 2.5 | 16 | 180 | 16.1 | 94 | 90 |
| I | | 4 | 13.5 | 180 | 15.1 | 94 | 95 |
| J | | 2.5 | 13.5 | 180 | 15.1 | 94 | 91 |

### Example 60

### IVT Platform Fed Batch Process Applications

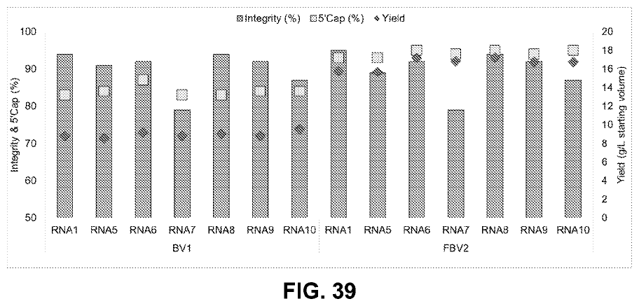

In this example, Process FB V2 was used to demonstrate general applicability, process robustness, and the successful application of this fed batch process across various RNA constructs of different backgrounds. The specific details of this FB V2 process are listed in Table 65. As shown in FIG. 39, all seven RNA constructs demonstrated markedly increased yields (>˜16 g/L), RNA integrity (>˜80%), and capping efficiency (>93%) in FB V2 compared to its BV1 counterpart.

TABLE 65
Feb Batch IVT Process Parameters

| Parameter | Setpoint | | | |
|---|---|---|---|---|
| Process Name | FB V1 | FB V1.1 | FB V1.2 | FB V2 |
| 5′Cap analog molecule (mM) | 4 | 4 | 4 | 4 |
| Initial ATP (mM) | 5 | 11 | 11 | 11 |
| Final ATP (mM) | 12 | 24 | 24 | 24 |
| Initial CTP (mM) | 5 | 9 | 9 | 9 |
| Final CTP (mM) | 12 | 22 | 22 | 22 |
| Initial GTP (mM) | 1 | 1 | 1 | 1 |
| Final GTP (mM) | 10 | 16 | 16 | 16 |
| Initial modified UTP (mM) | 5 | 4 | 4 | 4 |
| Final modified UTP (mM) | 9 | 15 | 13 | 13 |
| NTP Delivery Strategy | Varies | Boluses | Boluses | Boluses |
| | | every 5 min | every 5 min | every 5 min |
| Tris Buffer, pH 8.0 (mM) | 40 | 40 | 40 | 40 |
| HEPES Buffer, pH 8.0 (mM) | 0 | 0 | 0 | 0 |
| Starting Mg (mM) | 36 | 36 | 25 | 25 |
| Ending Mg (mM) | 36 | 36 | 60 | 60 |
| Mg Delivery Strategy | N/A | N/A | Separate bolus | Separate bolus |
| | | | every 15 | every 15 |
| | | | minutes for first | minutes for first |
| | | | 60 minutes | 60 minutes |
| Spermidine (mM) | 0 | 0 | 0 | 0 |
| DTT (mM) | 0 | 0 | 0 | 0 |
| Plasmid DNA Template | 0.1 | 0.1 | 0.1 | 0.1 |
| (mg/mL) | | | | |
| DNA Type | DNA1 | DNA1 | DNA1 | Varies |
| Rnase Inhibitor (U/mL) | 100 | 100 | 100 | 100 |
| Pyrophosphatase (U/mL) | 0.25 | 0.25 | 0.25 | 0.25 |
| T7 Polymerase (U/mL) | 14000 | 14000 | 14000 | 14000 |
| Total IVT Time (min) | 150 | 150 | 150 | 180 |
| Temperature (deg C.) | 37 | 37 | 37 | 37 |

Similarly, the process FB V2 was tested across several constructs with varying coding sequence lengths. As construct length increased, it was hypothesized that there would be more opportunities for premature T7 transcription termination and mRNA degradation. Therefore, it was of interest to determine whether the mRNA generated by FB V2 had acceptable integrity for long constructs. For all constructs up to 6 kb, integrity was above 80% as shown in Table 66, below. This demonstrates that FB V2 is a suitable platform process for a wide variety of constructs.

TABLE 66
Impact of Construct Length in Process FB V2

| | Construct Name | Yield (g/L starting | |
|---|---|---|---|
| Process | (kb) | volume) | Integrity (%) |
| FB V2 | RNA 1 (1.9) | 15.2 | 92 |
| | RNA 22 (4.3) | 14.4 | 80 |
| | RNA 23 (5.9) | 12.1 | 81 |

### Example 61

### Impact of Residual Nucleotides on Residual DNA Impurity

In this example, the impact of intensifying NTPs during IVT fed batch on residual DNA was determined. FB V1 was modulated with 0.05 U/mL pyrophosphatase and 150 RPM to create reactions with high residual DNA. The study was performed using automated bioreactor systems. Table 67-Table 69 show the impact on modulating the NTPs on modulated FB V1. Increasing levels of ATP from 12-22 mM trend with decreasing levels of residual DNA. Increasing levels of CTP and pUTP from 12-22 mM and 9-15 mM respectively show a further decrease in residual DNA. The results of this experiment demonstrate that higher residual levels of at least one NTP could be used to improve residual DNA impurity.

TABLE 67
Effect of Residual ATP on Product Quality

| | Total | | Residual | Yield (g/L | |
|---|---|---|---|---|---|
| | ATP | ResATP | DNA (ng/mg | starting | Integrity |
| Process | (mM) | (uM) | product) | volume) | (%) |
| FB V1 | 12 | 1647 | 438 | 10.2 | 95 |
| (0.05 U/mL pyro) | 17 | 5432 | 401 | 10.7 | 95 |
| (150 RPM) | 22 | 9132 | 370 | 10.3 | 94 |

TABLE 68
Effect of Residual CTP on Product Quality

| | Total | | Residual | Yield (g/L | |
|---|---|---|---|---|---|
| | CTP | ResCTP | DNA (ng/mg | starting | Integrity |
| Process | (mM) | (uM) | product) | volume) | (%) |
| FB V1 | 12 | 859 | 438 | 10.2 | 95 |
| (0.05 U/mL pyro) | 17 | 4460 | 162 | 9.7 | 93 |
| (150 RPM) | 22 | 8645 | 9 | 8.4 | 94 |

TABLE 69
Effect of Residual pUTP on Product Quality

| | Total | | Residual | Yield (g/L | |
|---|---|---|---|---|---|
| | pUTP | RespUTP | DNA (ng/mg | starting | Integrity |
| Process | (mM) | (uM) | product) | volume) | (%) |
| FB V1 | 9 | 1838 | 438 | 10.2 | 95 |
| (0.05 U/mL | | | | | |
| pyro) | | | | | |
| (150 RPM) | 15 | 6083 | 40 | 9.2 | 94 |

### Example 62

### Oligo(dT) mRNA Affinity Study

To further improve mRNA integrity, mRNA affinity chromatography steps were developed using POROS™ oligo (dT) 25 (THERMO FISHER®) and oligo (dT) 18 monolith (SARTORIUS BIA SEPARATIONS®) purification. Both steps bound mRNA in 10 mM Tris/500 mM KCl/0.1 mM EDTA, pH 7.2 and were eluted with 10 mM Tris/0.1 mM EDTA, pH 7.2. As shown in Table 70 below, mRNA integrity improved from 61% for non-affinity purified material up to 79% when purified by the affinity step.

TABLE 70
mRNA Affinity Chromatography

| Condition | Pool Integrity (%) |
|---|---|
| Non-Affinity Purified Pool | 61 |
| POROS oligo(dT)25 Elution Pool | 75 |
| Sartorius BIA Separations | 79 |
| oligo(dT)18 Monolith Elution Pool | |

mRNA affinity chromatography steps were further developed through evaluating an array of buffer components (e.g. NaPi, Tris, HEPES, KCl, NaCl, EDTA, etc.) at various salt concentrations to optimize integrity improvement and overall recovery. As shown in Table 71 below, processes between the POROS™ oligo (dT) 25 (THERMO FISHER®) and oligo (dT) 18 monolith (SARTORIUS BIA SEPARATIONS®) columns yielded comparable integrity improvement for HEPES, Tris, NaPi, KCL, and NaCl buffers containing buffers.

TABLE 71
Effect of Buffer Components on mRNA Integrity and Recovery

| | Resin | | | Monolith | |
|---|---|---|---|---|---|
| Run Description | Run 2 | Run 3 | Run 5 | Run 7 | Run 11 |
| Equilibration/ | 10 mM Tris + | 10 mM Tris + | 10 mM Tris + | 10 mM Tris + | 50 mM NaPi, |
| Load/Wash 1 | 500 mM KCl + | 500 mM KCl + | 500 mM KCI + | 500 mM KCl + | 400 mM NaCl, |
| Conditions | 0.1 mM EDTA, | 0.1 mM EDTA, | 0.1 mM EDTA, | 0.1 mM EDTA, | 5 mM EDTA, |
| | pH 7.2 | pH 7.2 | PH 7.2 | pH 7.2 | pH 7.0 |
| Wash 2 | | 10mM Tris + | | 50 mM NaPi, | 50 mM NaPi, |
| | | 750 mM KCl + | | 5 mM EDTA, | 5 mM EDTA, |
| | | 0.1 mM EDTA, | | pH 7.0 | pH 7.0 |
| | | pH 7.2 | | | |
| Mid Salt Wash | | 10 mM Tris + | 10 mM Tris + | | |
| | | 250 mM KCl + | 250 mM KCl + | | |
| | | 0.1 mM EDTA, | 0.1 mM EDTA, | | |
| | | pH 7.2 | pH 7.2 | | |
| Operation | 20 CV Linear | 30 CVsWash | 30 CV Linear | Step Elution | Step Elution |
| | Gradient from | to Elution | Gradient from | | |
| | Wash 1 To | | Wash 1 | | |
| | Elution | | To Elution | | |
| Elution | 10 mM Tris, | 10 mM Tris, | 10 mM Tris, | 10 mM Tris, | 10 mM HEPES, |
| | 0.1 mM EDTA, | 0.1 mM EDTA, | 0.1 mM EDTA, | pH 7.0 | pH 7.0 |
| | pH 7.2 | pH 7.2 | pH 7.2 | | |
| Strip | WFI | WFI | WFI | WFI | WFI |
| Load Integrity (%) | 60.9 | 53 | 62 | 71 | 63 |
| Load Integrity | NMT 3 | 8 | NMT 3 | NMT 3 | NMT 3 |
| DS-LMS (%) | | | | | |
| Elution Pool | 75 | 72 | 80 | 79 | 76 |
| Integrity (%) | | | | | |
| Elution Pool | NMT 3 | 5 | NMT 3 | NMT 3 | NMT 3 |
| Integrity | | | | | |
| DS-LMS (%) | | | | | |

To further improve integrity and determine process-related impurity removal (e.g. fragments, residual DNA, LMS, etc.), wash studies using an array of salts (e.g. NaCl, KCl, etc.), denaturants (Urea, Agrinine, etc.), and chelating agents (e.g. EDTA, citrate, etc.) were explored using a 2000 nucleotide modRNA by high throughput screening on 96 well plates containing oligo (dT) 25 resin. An oligo (dT) resin packed column was equilibrated with 10 mM Tris/500 mM KCl/0.1 mM EDTA, pH 7.2. mRNA was bound to the column and washed with 10 mM Tris/500 mM KCl/0.1 mM EDTA, pH 7.2. The resin was removed from the column and transferred to a 96-well plate by an automated liquid handling system. Each well was washed with Tris Buffer, pH 7.2 containing salts spanning the following ranges, shown in Table 72, below.

TABLE 72
Ranges of Wash Solutions Tested

| Intermediate Wash Solution | Concentration Tested | | Interaction |
|---|---|---|---|
| KCl | 50-1000 | mM | Ionic |
| NaCl | 50-1000 | mM | Ionic |
| MgCl₂ | 2-200 | mM | Ionic |
| Arginine | 50-1000 | mM | Ionic |
| | | | Hydrogen bonding |
| Urea | 50-2000 | mM | Hydrogen bonding |
| EDTA + 200 mM KCl | 1-200 | mM | Metal complexation |

Each condition was eluted using 10 mM Tris, 1 mM EDTA, PH 7.2. Elution pools with high yield were analyzed for residual DNA. Conditions with high log reduction value (LRV) of residual DNA were submitted for mRNA Integrity by fragment analyzer, as shown in Table 73 below:

TABLE 73
Residual DNA Analysis and mRNA Integrity

| | | DNA Log | | FA |
|---|---|---|---|---|
| Sample | ResDNA | Reduction | Integrity | Integrity |
| Name | (ng/mg) | (LR) | % | % DS-LMS |
| Load | 44839.44 | | 81.0 | 11.9 |
| 50 mM | 47.65 | 3.0 | 83.4 | 10.5 |
| Arginine | | | | |
| 1000 mM | 50.06 | 3.0 | 80.2 | 8.3 |
| Arginine | | | | |
| 200 mM EDTA, | 50.22 | 3.0 | 76.3 | NMT 3 |
| 200 mM KCl | | | | |
| 1 mM EDTA, | 53.11 | 2.9 | 76.0 | NMT 3 |
| 200 mM KCl | | | | |
| 50 mM KCl | 22.31 | 3.3 | 81.8 | 9.2 |
| 250 mM KCl | 44.48 | 3.0 | | |
| 2 mM MgCl2 | 61.96 | 2.9 | 66.9 | NMT 3 |
| 20 mM MgCl2 | 64.97 | 2.8 | 73.1 | NMT 3 |
| 50 mM NaCl | 55.58 | 2.9 | 83.7 | 10.5 |
| 1000 mM | 69.01 | 2.8 | 81.7 | 12.5 |
| NaCl | | | | |
| 2000 mM | 23.81 | 3.3 | 80.8 | 9.2 |
| Urea | | | | |
| 50 mM Urea | 70.14 | 2.8 | 81.1 | 9.4 |

From the plate screen, KCl, NaCl, Arginine, and Urea were determined to provide sufficient reduction of residual DNA, maintain or improve mRNA integrity, and, in some instances, reduce DS-LMS. These conditions were scaled to robocolumns, using the wash solution ranges shown in Table 74. Results of the residual DNA analysis and mRNA integrity are shown in Table 75.

TABLE 74
Ranges of Wash Solutions Tested in Robocolumns

| Intermediate Wash Solution | Concentration Tested | Interaction |
|---|---|---|
| KCl | 50-1000 mM | Ionic |
| NaCl | 50-1000 mM | Ionic |
| Arginine | 50-1000 mM | Ionic |
| | | Hydrogen bonding |
| Urea | 50-2000 mM | Hydrogen bonding |

TABLE 75
Residual DNA Analysis and mRNA Integrity in Robocolumns

| | Wash Salt | mRNA | DS | ResDNA | Log DNA |
|---|---|---|---|---|---|
| Wash | Concen- | Integrity | LMS | (ng DNA/mg | Reduction |
| Salt | tration | (%) | (%) | RNA) | (LR ng DNA) |
| Load | | 80.0 | 11.83 | 28636.37 | |
| Arginine | 50 | 80.8 | 12.23 | 57.31 | 3.0 |
| Arginine | 500 | 77.4 | 14.07 | 38.76 | 3.1 |
| Arginine | 1000 | 78.3 | 12.83 | 153.78 | 2.6 |
| KCl | 50 | 81.0 | 12.23 | 83.43 | 2.9 |
| KCl | 500 | 79.4 | 13.97 | 157.56 | 2.5 |
| KCl | 1000 | 79.7 | 13.17 | 999.14 | 1.8 |
| NaCl | 50 | 79.7 | 13.13 | 69.48 | 2.9 |
| NaCl | 500 | 81.3 | 12.83 | 173.65 | 2.4 |
| NaCl | 1000 | 81.5 | 13.2 | 1016.64 | 1.8 |
| Urea | 50 | 82.4 | 11.7 | 200.97 | 2.4 |
| Urea | 1000 | 83.9 | 10.9 | 62.63 | 2.9 |
| Urea | 2000 | 86.3 | 8.63 | 108.49 | 2.9 |

Conditions that resulted in greater reduction of residual DNA were scaled up to resin and monolith columns for both 2000 nucleotide (as shown in Table 76) and 10000 nucleotide (as shown in Table 77) mRNA. DS-LMS reduction was observed, and integrity was conserved or improved across conditions. NaCl and KCl observed similar performance and only KCl was carried forward for the study. From the plate screen, EDTA demonstrated a potential improvement to the process, so an EDTA design leg was added to the experiment.

TABLE 76
Residual DNA and mRNA Analysis for 2000 nucleotide mRNA

| | mRNA Size | | | | | |
|---|---|---|---|---|---|---|
| | 2000 nT mRNA | | | | | |
| | Resin or Monolith | | | | | |
| | Resin | | | | | |
| | Method Step | | | | | |
| | A | B | C | D | E | F |
| Equilibration/Load | 10 mM Tris, 500 mM KCl, 20 mM EDTA, pH 7.2 | | | | | |
| Conditions | | | | | | |
| Wash | 50 mM | 50 mM | 1000 mM | 1000 mM | 50 mM | 50 mM |
| | Arginine | Arginine, | Urea | Urea, | KCl | KCl, |
| | | 20 mM | | 20 mM | | 20 mM |
| | | EDTA | | EDTA | | EDTA |
| Elution | 10 mM Tris, 1 mM EDTA, pH 7.2 | | | | | |
| Load Integrity (%) | 80 | 80 | 80 | 80 | 80 | 80 |
| Load Integrity DS- | 12 | 12 | 12 | 12 | 12 | 12 |
| LMS (%) | | | | | | |
| Load Residual | 22441 | 22441 | 22441 | 22441 | 22441 | 22441 |
| DNA(ng/mg) | | | | | | |
| Elution Pool | | 82 | 86 | 82 | 76 | 81 |
| Integrity (%) | | | | | | |
| Elution Integrity | | 12 | 8 | 12 | 11 | 12 |
| DS-LMS (%) | | | | | | |
| Elution Pool | 337 | 227 | 557 | 252 | 281 | 229 |
| Residual DNA | | | | | | |
| (ng/mg) | | | | | | |
| Residual DNA LRV | 2.02 | 2.22 | 1.96 | 2.11 | 2.17 | 2.11 |

TABLE 77
Residual DNA and mRNA Analysis for 10000 nucleotide mRNA

| | mRNA Size | | | | | | |
|---|---|---|---|---|---|---|---|
| | 10000 nt mRNA | | | | | | |
| | Resin or Monolith | | | | | | |
| | Resin | | | | | | |
| | Method Step | | | | | | |
| | A | B | C | D | E | F | G |
| Equilibration/ | 10 mM Tris, 500 mM KCl, 20 mM | | | | | | 50 mM Na |
| Load | EDTA, pH 7.2 | | | | | | Pi, 400 mM |
| Conditions | | | | | | | Na Cl, 5 mM |
| | | | | | | | EDTA, pH 6.5 |
| Wash | 50 mM | 50 mM | 1000 mM | 1000 mM | 50 mM | 50 mM | 50 mM Na |
| | Arginine | Arginine, | Urea, | Urea, | KCl | KCl, | Pi, 5 mM |
| | | 20 mM | | 20 mM | | 20 mM | EDTA, pH 7.0 |
| | | EDTA | | EDTA | | EDTA | |
| Elution | 10 mM HEPES, 0.1 mM EDTA, pH 7.1 | | | | | | |
| Load | 76 | 76 | 76 | 76 | 76 | 76 | 75 |
| Integrity | | | | | | | |
| (%) | | | | | | | |
| Load | NMT | NMT | NMT | NMT | NMT | NMT | NMT |
| Integrity | 3 | 3 | 3 | 3 | 3 | 3 | 3 |
| DS-LMS | | | | | | | |
| (%) | | | | | | | |
| Load | 8015 | 8015 | 8015 | 8015 | 8015 | 8015 | 8015 |
| Residual | | | | | | | |
| DNA(ng/mg) | | | | | | | |
| Elution | | 86 | 86 | 88 | 85 | 88 | 88 |
| Pool | | | | | | | |
| Integrity | | | | | | | |
| (%) | | | | | | | |
| Elution | | NMT | NMT | NMT | NMT | NMT | NMT |
| Integrity | | 3 | 3 | 3 | 3 | 3 | 3 |
| DS-LMS | | | | | | | |
| (%) | | | | | | | |
| Elution | 364 | 289 | 338 | 315 | 324 | 335 | |
| Pool | | | | | | | |
| Residual | | | | | | | |
| DNA | | | | | | | |
| (ng/mg) | | | | | | | |
| Residual | 1.34 | 1.44 | 1.37 | 1.41 | 1.39 | 1.38 | |
| DNA | | | | | | | |
| LRV | | | | | | | |

| | mRNA Size | | | | | |
|---|---|---|---|---|---|---|
| | 10000 nt mRNA | | | | | |
| | Resin or Monolith | | | | | |
| | Monolith | | | | | |
| | Method Step | | | | | |
| | A | B | C | D | E | F |
| Equilibration/ | 10 mM Tris, 400 mM NaCl, 5 mM | | | | | |
| Load | EDTA, pH 7.2 | | | | | |
| Conditions | | | | | | |
| Wash | 50 mM | 50 mM | 1000 mM | 1000 mM | 50 mM | 50 mM |
| | Arginine | Arginine, | Urea | Urea, | KCl | KCl, |
| | | 20 mM | | 20 mM | | 20 mM |
| | | EDTA | | EDTA | | EDTA |
| Elution | 10 mM HEPES, 0.1 mM EDTA, pH 7.1 | | | | | |
| Load | 76 | 76 | 76 | 76 | 76 | 76 |
| Integrity | | | | | | |
| (%) | | | | | | |
| Load | NMT | NMT | NMT | NMT | NMT | NMT |
| Integrity | 3 | 3 | 3 | 3 | 3 | 3 |
| DS-LMS | | | | | | |
| (%) | | | | | | |
| Load | 8015 | 8015 | 8015 | 8015 | 8015 | 8015 |
| Residual | | | | | | |
| DNA(ng/mg) | | | | | | |
| Elution | | 83 | 87 | 86 | 87 | 84 |
| Pool | | | | | | |
| Integrity | | | | | | |
| (%) | | | | | | |
| Elution | | NMT | NMT | NMT | NMT | NMT |
| Integrity | | 3 | 3 | 3 | 3 | 3 |
| DS-LMS | | | | | | |
| (%) | | | | | | |
| Elution | 300 | 334 | 258 | 345 | 321 | 407 |
| Pool | | | | | | |
| Residual | | | | | | |
| DNA | | | | | | |
| (ng/mg) | | | | | | |
| Residual | 1.43 | 1.38 | 1.49 | 1.37 | 1.4 | 1.29 |
| DNA | | | | | | |
| LRV | | | | | | |

For 2000 nucleotide mRNA, 2 log reduction value (LRV) reduction was observed across all conditions. For 10000 nucleotide mRNA, 1.3-1.5 LRV was observed across conditions. While the reduction by LRV calculation was less for the 10000 nucleotide mRNA, the absolute levels of residual DNA in the elution pools were the same irrespective of mRNA length. The lower LRV may be an artifact of significantly lower residual DNA in the load for the 10000 nucleotide. The lower residual DNA in the load is intrinsic to the IVT process, which utilizes lower DNA template the 2000 nucleotide process. Some DS-LMS reduction was observed for the 1000 mM Urea condition in the 2000 nucleotide mRNA. Across mRNA lengths, 1000 mM Urea condition for resin and monolith resulted in one of the highest integrity improvements. Generally, comparable integrity improvement was observed between monolith and resin for the 10000 nucleotide mRNA. Condition A resulted in samples that could not return mRNA integrity data. Condition G was added to the resin arm of experimentation to compare NaPi and NaCl to the Tris and KCl process with respect to integrity improvement. The results yielded that the integrity improvement was identical between the conditions.

Double-stranded RNA (dsRNA) was measured at the load and elution pools for the 10000 nucleotide mRNA for conditions A-G, shown in Table 78 below. Some reduction was observed, but was not significantly reduced. It is well known that dsRNA from IVT mixtures are made up of either truncated/abortive species forming complementary base-pairs with other mRNA or turnaround transcription. In both instances, it is possible that the dsRNA has poly-A tail, and would therefore still bind. The slight reduction across conditions indicates that removing the non-poly-A-tail impurities also reduced some of the dsRNA as measured by the method. The further reduction observed for Urea+EDTA conditions indicates that disruption of the hydrogen bonding between abortive/truncated species and complementary mRNA may be key. The relationship between temperature and hydrogen bonding is well understood for double stranded nucleic acids in oligonucleotides, specifically, that as temperature increases, the hydrogen bonds between complimentary base pairs of double stranded species weakens, resulting in conversion into single stranded species. Future experimentation would expect that a wash step that is optimized for buffer composition and temperature would result in removal of double stranded species while retaining the target mRNA.

TABLE 78
dsRNA presence

dsRNA - ELISA
(pg/mg)

| Condition | RESIN | MONOLITH |
|---|---|---|
| Oligo(dT) Load | 3174 | 3174 |
| 50 mM Arginine** | 2797 | 3289 |
| 50 mM Arginine, 20 mM EDTA | 2700 | 2703 |
| 1000 mM Urea | 2234 | 2540 |
| 1000 mM Urea, 20 mM EDTA | 2503 | 2442 |
| 50 mM KCl* | 2520 | 2661 |
| 50 mM KCl, 20 mM EDTA | 2703 | 2594 |

The codeveloped processes for POROS™ oligo (dT) 25 (THERMO FISHER®) and oligo (dT) 18 monolith (SARTORIUS BIA SEPARATIONS®) for mRNA lengths ranging 2000-10000 nucleotides identified that buffers composing of combinations of Tris, HEPES, NaPi, KCl, NaCl, Urea, Arginine, and EDTA, result in integrity improvement and/or removal of residual DNA.

The examples and embodiments described herein are for illustrative purposes only and various modifications or changes suggested to person skilled in the art are to be included within the spirit and purview of this application and scope of the appended claims.

1. A method of producing an mRNA molecule comprising:
(a) obtaining a composition comprising:
a. a circular double-stranded DNA (dsDNA) template,
b. a primer,
c. a deoxyribonucleotide triphosphate (dNTP), and
d. a DNA polymerase;
(b) incubating the composition for a time period between about three and thirty hours at a temperature between about 30° C. and 45° C. to obtain an incubated composition; and
(c) contacting the incubated composition with an in vitro transcription reaction system comprising an RNA polymerase and ribonucleotides under conditions sufficient for in vitro transcription to produce the mRNA molecule,
wherein the contacting occurs for between about 120 minutes and about 260 minutes,
wherein the contacting occurs at a temperature between 25° C. and 45° C., wherein the in vitro transcription reaction system further comprises a buffer and a magnesium ion, and
wherein the in vitro transcription reaction system lacks a polyamine.

2. The method of claim 1, further comprising contacting the incubated composition with a restriction endonuclease to obtain a digested composition prior to contacting the incubated composition with the in vitro transcription reaction system.

3. The method of claim 2, wherein contacting the incubated composition with the restriction endonuclease occurs in a same reaction vessel as incubating the composition.

4. The method of any one of claims 1-3, wherein contacting the incubated composition with the in vitro transcription reaction system occurs in the same reaction vessel as incubating the composition.

5. The method of any one of claims 1-4, wherein the method does not comprise a heat denaturation reaction prior to incubating the composition.

6. The method of any one of claims 1-5, wherein the method does not comprise a heat inactivation reaction prior to performing the in vitro transcription reaction.

7. The method of any one of claims 1-6, further comprising:
(d) capturing the mRNA molecule using a capture method selected from the group consisting of an oligo (dT) magnetic bead, a resin, and a monolith.

8. The method of any one of claims 1-7, wherein the time period is between about three and eight hours.

9. The method of any one of claims 1-7, wherein the time period is between about eighteen and twenty-four hours.

10. The method of any one of claims 1-9, wherein the DNA polymerase is a phi29 DNA polymerase.

11. The method of any one of claims 1-10, wherein contacting further comprises supplementing the in vitro transcription reaction system with ribonucleotides.

12. The method of claim 11, wherein the supplementing comprises continuous feeding of ribonucleotides.

13. The method of claim 11, wherein the supplementing comprises semi-continuous feeding of ribonucleotides.

14. The method of claim 11, wherein the supplementing comprises bolus feeding of ribonucleotides.

15. The method of any one of claims 1-14, wherein contacting further comprises supplementing the in vitro transcription reaction system with a magnesium ion.

16. The method of claim 15, wherein the supplementing is selected from the group consisting of continuous feeding, semi-continuous feeding, and bolus feeding of a magnesium ion.

17. The method of any one of claims 1-17, wherein the contacting further comprises agitation.

18. The method of claim 17, wherein the agitation is selected from the group consisting of a power/volume between about 1.3 and about 71.7 W/m³, a mixing time between about 1.3 and about 12.1 seconds, and an impeller tip speed between about 0.1 and about 0.4 m/s.

19. The method of any one of claims 1-18, wherein the in vitro transcription reaction system further comprises pyrophosphatase.

20. The method of any one of claims 1-19, wherein the in vitro transcription reaction system further comprises a ribonuclease inhibitor.

21. The method of any one of claims 1-20, wherein the in vitro transcription reaction system further comprises an acetate ion.

22. The method of any one of claims 1-21, wherein the buffer is selected from the group consisting of TRIS and HEPES.

23. The method of any one of claims 1-22, wherein the ribonucleotides are present in an amount between about 16 and about 50 mM.

24. The method of any one of claims 1-23, wherein the RNA polymerase is present in an amount between about 4000 and about 12000 U/mL.

25. The method of claim 19, wherein the pyrophosphatase is present in an amount between about 0.25 and about 8.0 U/mL.

26. The method of any one of claims 1-25, wherein the buffer comprises a starting pH between about 7.5 and about 8.5.

27. The method of any one of claims 1-26, wherein the magnesium ion is present in an amount between about 12.8 and about 110 mM.

28. The method of any one of claims 1-27, wherein the magnesium ion and the ribonucleotides are present in a magnesium:ribonucleotide ratio of about 0.8 to about 2.2 mM Mg/mM NTP.

29. The method of any one of claims 1-28, wherein the in vitro transcription reaction system lacks a reducing agent.

30. The method of claim 29, wherein the reducing agent is dithiothreitol (DTT).

31. A method of producing an mRNA molecule comprising:
(a) obtaining a composition comprising:
a. a plurality of linear double-stranded DNA (dsDNA) fragments,
b. a 5′ exonuclease,
c. a DNA polymerase, and
d. a DNA ligase;
(b) incubating the composition at a temperature between about 45° C. and 55° C. to obtain a circularized dsDNA template; and
(c) contacting the circularized dsDNA template with a rolling circle amplification (RCA) reaction mix comprising:
a. a primer or a primase enzyme,
b. a deoxyribonucleotide triphosphate (dNTP), and
c. a DNA polymerase
to obtain an amplified template;
(d) contacting the amplified template with a restriction enzyme to obtain a digested template; and
(e) contacting the digested template with an in vitro transcription reaction system comprising an RNA polymerase and ribonucleotides under conditions sufficient for in vitro transcription to produce the mRNA molecule,
wherein the contacting occurs for between about 120 minutes and about 260 minutes,
wherein the contacting occurs at a temperature between 25° C. and 45° C.,
wherein the in vitro transcription reaction system further comprises a buffer and a magnesium ion, and
wherein steps (a) through (e) are performed sequentially in a single reaction vessel.

32. The method of claim 31, wherein the circularized dsDNA template is present at a concentration of at least 0.5 ng per mL of RCA reaction mix.

33. The method of claim 31, wherein the plurality of linear dsDNA fragments comprises a gene of interest, a promoter sequence, a 5′ UTR, a 3′ UTR, and a poly A sequence.

34. The method of claim 33, wherein the gene of interest encodes an RNA molecule between 1.0 kb and 12.0 kb.

35. The method of claim 33, wherein the gene of interest lacks homopolymer sequences greater than 5 base pairs.

36. The method of claim 31, wherein the mRNA molecule is capped following in vitro transcription.

37. The method of claim 36, wherein the mRNA molecule is capped enzymatically by a vaccinia virus capping enzyme.

38. The method of claim 37, wherein the mRNA molecule is incubated at a temperature of 30° C. or lower for 60 minutes or less prior to capping by the vaccinia virus capping enzyme.

39. The method of claim 31, wherein contacting the digested template with an in vitro transcription reaction system further comprises supplementing the in vitro transcription reaction system with ribonucleotides, wherein supplementing comprises increasing a concentration of an individual ribonucleotide from an initial amount to a final amount.

40. The method of claim 39, wherein supplementing comprises a method selected from the group consisting of continuous feeding of ribonucleotides, semi-continuous feeding of ribonucleotides, and bolus feeding of ribonucleotides.

41. The method of claim 39, wherein the initial amount is between 1 mM and 11 mM and the final amount is between 11 mM and 26 mM.

42. The method of claim 41, wherein the initial amount of ATP is about 11 mM, the initial amount of CTP is about 9 mM, the initial amount of GTP is about 1 mM, and the initial amount of pUTP is about 4 mM.

43. The method of claim 42, wherein the final amount of ATP is about 24 mM, the final amount of CTP is about 22 mM, the final amount of GTP is about 16 mM, and the final amount of pUTP is about 13 mM.

44. The method of claim 43, wherein the supplementing further comprises bolus feeding of ribonucleotides every five minutes of the in vitro transcription reaction.

45. The method of claim 31, wherein contacting the digested template with an in vitro transcription reaction system further comprises supplementing the in vitro transcription reaction system with a magnesium ion, wherein supplementing comprises increasing a concentration of the magnesium ion from an initial amount to a final amount.

46. The method of claim 45, wherein supplementing comprises a method selected from the group consisting of continuous feeding of a magnesium ion, semi-continuous feeding of a magnesium ion, and bolus feeding of a magnesium ion.

47. The method of claim 46, wherein supplementing the in vitro transcription reaction system with a magnesium ion further comprises at least four magnesium ion additions during the in vitro transcription reaction.

48. The method of claim 45, wherein the initial amount of the magnesium ion is about 25 mM and the final amount of the magnesium ion is about 60 mM.

49. The method of claim 48, wherein the supplementing further comprises bolus feeding of the magnesium ion every 15 minutes for the first 60 minutes of the in vitro transcription reaction.

50. The method of any of claims 31-49, further comprising.
(f) capturing the mRNA molecule using a capture method selected from the group consisting of an oligo (dT) magnetic bead, a resin, and a monolith.

51. The method of claim 50, further comprising washing the capture method with a wash solution following capturing the mRNA.

52. The method of claim 51, wherein the wash solution comprises a buffer selected from the group consisting of Tris, HEPES, NaPi, KCl, NaCl, Urea, Arginine, and EDTA.
